(19) United States
(12) Patent Application Publication
(10) Pub. No.: US 2008/0175900 A1
(43) Pub. Date: Jul. 24, 2008

(54) PHARMACEUTICAL FORMULATIONS OF AN ANHYDRATE CRYSTAL FORM OF 2-(3-FLUORO-4-HYDROXYPHENYL)-7-VINYL-1,3-BENZOXAZOL-5-OL

(75) Inventors: Mahesh K. KRISHNAN, Stony Point, NY (US); Mohamed GHORAB, Nasr City (EG); Rolland W. CARSON, Tomkins Cove, NY (US); Patricia Carson, Roswell, GA (US); Shamim HASAN, East Elmhurst, NY (US); Arwinder NAGI, Thiells, NY (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/942,798

(22) Filed: Nov. 20, 2007

Related U.S. Application Data
(60) Provisional application No. 60/860,317, filed on Nov. 21, 2006.

(51) Int. Cl. A61K 9/48; A61K 31/423

(52) U.S. Cl. 424/455; 514/375

(57) ABSTRACT

The present invention is directed to pharmaceutical formulations of an anhydrate crystal form of an estrogen receptor modulator, and pharmaceutical compositions and preparative processes thereof.

This application claims benefit of priority to U.S. provisional patent application Ser. No. 60/860,317 filed Nov. 21, 2006, which is hereby incorporated by reference in its entirety.

### FIELD OF THE INVENTION

The present invention is directed to pharmaceutical formulations and compositions of an anhydrate crystal form of an estrogen receptor modulator (Form B of ERB-041), and processes for their preparation.

### BACKGROUND OF THE INVENTION

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Women's Health & Gender Based Medicine 8: 1155-1166 (1999), Monk and Brodaty, Dementia & Geriatric Cognitive Disorders 11: 1-10 (2000), Hurn and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001), each of which is hereby incorporated by reference in its entirety]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles of Molecular Regulation 351-361 (2000), which is hereby incorporated by reference in its entirety]. A class of “coregulatory” proteins can also interact with the ligand-bound receptor and further modulate its transcriptional activity [McKenna, et al., Endocrine Reviews 20: 321-344 (1999), which is hereby incorporated by reference in its entirety]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001), each of which is hereby incorporated by reference in its entirety].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001), which is hereby incorporated by reference in its entirety].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999), which is hereby incorporated by reference in its entirety].

Two estrogen receptors have been discovered to date. The first estrogen receptor was cloned about 15 years ago and is now referred to as ERα [Green, et al., Nature 320: 134-9 (1986), which is hereby incorporated by reference in its entirety]. The second form of the estrogen receptor was found comparatively recently and is called ERβ [Kuiper, et al., Proceedings of the National Academy of Sciences of the United States of America 93: 5925-5930 (1996), which is hereby incorporated by reference in its entirety]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997), which is hereby incorporated by reference in its entirety]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999), which is hereby incorporated by reference in its entirety]. However, there are examples where the receptors are coexpressed and there is evidence from in vitro studies that ERα and ERβ can form heterodimers [Cowley, et al., Journal of Biological Chemistry 272: 19858-19862 (1997), which is hereby incorporated by reference in its entirety].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA®) [McDonnell, Journal of the Society for Gynecologic Investigation 7: S10-S15 (2000), Goldstein, et al., Human Reproduction Update 6: 212-224 (2000), which is hereby incorporated by reference in its entirety]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist that sterically hinders the protein sequences required for receptor-coregulatory protein interaction [Pike, et al., EMBO 18: 4608-4618 (1999), Shiau, et al., Cell 95: 927-937 (1998), which is hereby incorporated by reference in its entirety]. In addition, the technique of phage display has been used to identify peptides that interact with estrogen receptors in the presence of different ligands [Paige, et al., Proceedings of the National Academy of Sciences of the United States of America 96: 3999-4004 (1999), which is hereby incorporated by reference in its entirety]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

Given the importance of estrogen receptor modulators in affecting a panoply of biological processes, there is an interest in developing new ERβ selective ligands and pharmaceutical formulations and compositions thereof. To this end, exemplary ERβ selective ligands, including 2-(3-fluoro-4-hydroxyphenyl)-7-vinyl-1,3-benzoxazol-5-ol (ERB-041), are described in U.S. Pat. No. 6,794,403, hereby incorporated by reference in its entirety including 2-(3-fluoro-4-hydroxyphenyl)-7-vinyl-1,3-benzoxazol-5-ol (ERB-041), is described in U.S. Pat. No. 6,794,403, hereby incorporated by reference in its entirety. The anhydrate crystal form (Form B) of ERB-041 used in the formulations of the present invention has been disclosed in a U.S. provisional patent application (U.S. provisional patent application Ser. No. 60/860,265 filed Nov. 21, 2006), which is incorporated by reference herein in its entirety. Additionally, two different crystal forms of ERB-041, a monohydrate and an anhydrous crystal form, have been disclosed in U.S. Provisional Patent Application No. 60/659,459 filed Mar. 8, 2005, which is incorporated by reference herein in its entirety. Moreover, three additional different crystal forms of 2-(3-fluoro-4-hydroxyphenyl)-7-vinyl-1,3-benzoxazol-5-ol, two anhydrate crystal forms and another crystal form, have been disclosed in three U.S. provisional patent applications (U.S. provisional patent application Ser. No. 60/860,253 filed Nov. 21, 2006; U.S. provisional patent application Ser. No. 60/860,246 filed Nov. 21, 2006; and U.S. provisional patent application Ser. No. 60/860,248 filed Nov. 21, 2006), each of which is hereby incorporated by reference in its entirety. In addition, formulations for these three additional different crystal forms have been disclosed in three U.S. provisional patent applications (U.S. provisional patent application Ser. No. 60/860,318 filed Nov. 21, 2006; U.S. provisional patent application Ser. No. 60/860,352 filed Nov. 21, 2006; and U.S. provisional patent application Ser. No. 60/860,415 filed Nov. 21, 2006), each of which is hereby incorporated by reference in its entirety.

It is well known that the crystal form of a particular drug is often an important determinant of the drug's ease of preparation, stability, solubility, storage stability, ease of formulation and in vivo pharmacology. Different crystal forms occur where the same composition of matter crystallizes in a different lattice arrangement resulting in different thermodynamic properties and stabilities specific to the particular polymorph form. In cases where two or more crystal forms can be produced, it is desirable to have a method to make both crystal forms in pure form. In deciding which crystal form is preferable, the numerous properties of the crystal forms must be compared and the preferred crystal form chosen based on the many physical property variables. It is entirely possible that one crystal form can be preferable in some circumstances where certain aspects such as ease of preparation, stability, etc. are deemed to be critical. In other situations, a different crystal form maybe preferred for greater solubility and/or superior pharmacokinetics.

Because of the potential advantages associated with one pure crystal form, it is desirable to prevent or minimize polymorphic conversion (i.e., conversion of one crystal form to another; or conversion between one crystal form and amorphous form) when two or more crystal forms of one substance can exist. Such polymorphic conversion can occur during both the preparation of formulations containing the crystal form, and during storage of a pharmaceutical dosage form containing the crystal form.

Given the potential advantages of a single crystal form, it can be seen that formulations having reduced polymorphic conversion can provide significant benefits. The 2-(3-fluoro-4-hydroxyphenyl)-7-vinyl-1,3-benzoxazol-5-ol formulations and compositions described herein help meet these and other needs.

### DESCRIPTION OF THE FIGURES

FIG. 1 depicts X-Ray powder diffraction (XRPD) pattern for the anhydrate crystal form (Form B) used in the formulations of the present invention.

FIG. 2 depicts a differential scanning calorimetry (DSC) thermogram of the anhydrate crystal form (Form B) used in the formulations of the present invention.

FIG. 3 depicts a thermogravimetric analysis (TGA) of the anhydrate crystal form (Form B) used in the formulations of the present invention.

### SUMMARY OF THE INVENTION

In one aspect, the present invention provides liquid or semi-solid pharmaceutical formulations comprising:

(a) a first carrier component comprising from about 10% to about 99.99% by weight of the pharmaceutical formulation;

(b) an optional second carrier component, when present, comprising up to about 70% by weight of the pharmaceutical formulation;

(c) an optional emulsifying/solubilizing component, when present, comprising from about 0.01% to about 30% by weight of the pharmaceutical formulation;

(d) an optional anti-crystallization/solubilizing component, when present, comprising from about 0.01% to about 30% by weight of the pharmaceutical formulation; and

(e) an active pharmacological agent comprising from about 0.01% to about 80% of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol.

The present invention further provides liquid or semi-solid pharmaceutical formulations comprising:

(a) a first carrier component comprising from about 10% to about 99.99% by weight of the pharmaceutical formulation;

(b) an optional second carrier component, when present, comprising up to about 70% by weight of the pharmaceutical formulation;

(c) an emulsifying/solubilizing component comprising from about 0.01% to about 30% by weight of the pharmaceutical formulation;

(d) an optional anti-crystallization/solubilizing component, when present, comprising from about 0.01% to about 30% by weight of the pharmaceutical formulation; and

(e) an active pharmacological agent comprising from about 0.01% to about 80% of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol.

The present invention further provides a process for preparing the liquid or semi-solid pharmaceutical formulations of the invention comprising mixing the first carrier component and the active pharmaceutical agent with sufficient heating to obtain a suspension or solution of the active pharmaceutical agent.

The present invention further provides hard gel or soft gel capsule comprising the liquid or semi-solid pharmaceutical formulations of the invention.

In another aspect, the present invention provides solid pharmaceutical formulations comprising:

(a) a first diluent/filler component comprising from about 30% to about 95% by weight of the formulation;

(b) an optional second diluent/filler component, when present, comprising up to about 40% by weight of the pharmaceutical formulation;

(c) a disintegrant component comprising from about 0.01% to about 30% by weight of the pharmaceutical formulation;

(d) a binder component comprising from about 0.01% to about 20% by weight of the pharmaceutical formulation;

(e) a wetting agent component comprising from about 0.01% to about 20% by weight of the pharmaceutical formulation;

(f) an optional lubricant component, when present, comprising from about 0.01% to about 10% by weight of the pharmaceutical formulation; and

(g) an active pharmacological agent comprising from about 0.01% to about 80% by weight of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol.

The present invention further provides a process for preparing the solid pharmaceutical formulations of the invention comprising:

(a) mixing the active pharmacological agent with the first diluent/filler component, the disintegrant component, and the optional second filler/diluent component, if present, to form an initial mixture; and

(b) granulating the initial mixture with an aqueous solution comprising the wetting agent component to form a granulated mixture.

The present invention further provides a process for preparing the solid pharmaceutical formulations of the invention comprising:

(i) mixing the active pharmacological agent with at least a portion of the first diluent/filler component to form a first mixture;

(ii) mixing the first mixture with the remainder of the first diluent/filler component, if any, the disintegrant component, and the optional second filler/diluent component, if present, to form the initial mixture;

(iii) granulating the initial mixture with an aqueous solution comprising the wetting agent component to form a granulated mixture

(iv) drying the granulated mixture to form a dried granulated mixture;

(v) mixing the optional lubricant component, if present, with the at least a portion of the dried granulated mixture; and

(vi) mixing the mixture from (v) with the remainder of the dried granulated mixture, if any.

The present invention further provides a process for producing the solid pharmaceutical formulations of the invention comprising:

(i) mixing the first diluent/filler component, the optional second diluent/filler component, if present, the disintegrant component, the binder component, the wetting agent component, and the active pharmacological agent to form a first mixture; and

ii) optionally granulating the first mixture.

The present invention further provides tablets comprising the pharmaceutical formulations of the invention.

The present invention further provides a process for producing the tablets of the invention comprising compressing the pharmaceutical formulations of the invention.

The present invention further provides products of the processes of the invention.

### DETAILED DESCRIPTION

### Liquid or Semi-Solid Pharmaceutical Formulations

The present invention is directed to pharmaceutical formulations of a specific anhydrate crystal form (Form B) of 2-(3-fluoro-4-hydroxyphenyl)-7-vinyl-1,3-benzoxazol-5-ol (ERB-041). Accordingly, in one aspect, the present invention provides liquid or semi-solid pharmaceutical formulations comprising:

(a) a first carrier component comprising from about 10% to about 99.99% by weight of the pharmaceutical formulation;

(b) an optional second carrier component, when present, comprising up to about 70% by weight of the pharmaceutical formulation;

(c) an optional emulsifying/solubilizing component, when present, comprising from about 0.01% to about 30% by weight of the pharmaceutical formulation;

(d) an optional anti-crystallization/solubilizing component, when present, comprising from about 0.01% to about 30% by weight of the pharmaceutical formulation; and

(e) an active pharmacological agent comprising from about 0.01% to about 80% of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol.

The present invention further provides liquid or semi-solid pharmaceutical formulations comprising:

(a) a first carrier component comprising from about 10% to about 99.99% by weight of the pharmaceutical formulation;

(b) an optional second carrier component, when present, comprising up to about 70% by weight of the pharmaceutical formulation;

(c) an emulsifying/solubilizing component comprising from about 0.01% to about 30% by weight of the pharmaceutical formulation;

(d) an optional anti-crystallization/solubilizing component, when present, comprising from about 0.01% to about 30% by weight of the pharmaceutical formulation; and

(e) an active pharmacological agent comprising from about 0.01% to about 80% of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol.

In some embodiments:

(a) the first carrier component comprises from about 30% to about 90% by weight of the pharmaceutical formulation;

(b) the optional second carrier component, when present, comprises up to about 50% by weight of the pharmaceutical formulation;

(c) the emulsifying/solubilizing component comprises from about 0.1% to about 20% by weight of the pharmaceutical formulation;

(d) the optional anti-crystallization/solubilizing component, when present, comprises from about 0.1% to about 20% by weight of the pharmaceutical formulation; and

(e) the active pharmacological agent comprises from about 0.1% to about 50% by weight of the pharmaceutical formulation.

In some embodiments:

(a) the first carrier component comprises from about 50% to about 90% by weight of the pharmaceutical formulation;

(b) the optional second carrier component, when present, comprises up to about 30% by weight of the pharmaceutical formulation;

(c) the emulsifying/solubilizing component comprises from about 0.1% to about 10% by weight of the pharmaceutical formulation;

(d) the optional anti-crystallization/solubilizing component, when present, comprises from about 0.1% to about 20% by weight of the pharmaceutical formulation; and

(e) the active pharmacological agent comprises from about 0.1% to about 50% by weight of the pharmaceutical formulation.

In some embodiments:

(a) the first carrier component comprises from about 50% to about 70% by weight of the pharmaceutical formulation;

(b) the optional second carrier component, when present, comprises up to about 30% by weight of the pharmaceutical formulation;

(c) the emulsifying/solubilizing component comprises from about 0.1% to about 10% by weight of the pharmaceutical formulation;

(d) the optional anti-crystallization/solubilizing component, when present, comprises from about 0.1% to about 15% by weight of the pharmaceutical formulation; and

(e) the active pharmacological agent comprises from about 0.1% to about 40% by weight of the pharmaceutical formulation.

In some embodiments:

(a) the first carrier component comprises from about 30% to about 50% by weight of the pharmaceutical formulation;

(b) the optional second carrier component, when present, comprises from about 30% to about 50% by weight of the pharmaceutical formulation;

(c) the emulsifying/solubilizing component comprises from about 0.1% to about 10% by weight of the pharmaceutical formulation;

(d) the optional anti-crystallization/solubilizing component, when present, comprises from about 0.1% to about 15% by weight of the pharmaceutical formulation; and

(e) the active pharmacological agent comprises from about 0.1% to about 40% by weight of the pharmaceutical formulation.

In some embodiments:

(a) the first carrier component comprises from about 65% to about 85% by weight of the pharmaceutical formulation;

(b) the optional second carrier component, when present, comprises up to about 30% by weight of the pharmaceutical formulation;

(c) the emulsifying/solubilizing component comprises from about 0.1% to about 10% by weight of the pharmaceutical formulation;

(d) the optional anti-crystallization/solubilizing component, when present, comprises from about 0.1% to about 15% by weight of the pharmaceutical formulation; and

(e) the active pharmacological agent comprises from about 0.1% to about 40% by weight of the pharmaceutical formulation.

In some embodiments:

(a) the first carrier component comprises from about 65% to about 85% by weight of the pharmaceutical formulation;

(b) the optional second carrier component, when present, comprises from about 5% to about 15% by weight of the pharmaceutical formulation;

(c) the emulsifying/solubilizing component comprises from about 0.1% to about 10% by weight of the pharmaceutical formulation;

(d) the optional anti-crystallization/solubilizing component, when present, comprises from about 0.1% to about 15% by weight of the pharmaceutical formulation; and

(e) the active pharmacological agent comprises from about 0.1% to about 40% by weight of the pharmaceutical formulation.

In some embodiments:

(a) the first carrier component comprises from about 50% to about 90% by weight of the pharmaceutical formulation;

(b) the optional second carrier component, when present, comprises up to about 30% by weight of the pharmaceutical formulation;

(c) the emulsifying/solubilizing component comprises from about 1% to about 10% by weight of the pharmaceutical formulation;

(d) the optional anti-crystallization/solubilizing component, when present, comprises from about 1% to about 10% by weight of the pharmaceutical formulation; and

(e) the active pharmacological agent comprises from about 1% to about 25% by weight of the pharmaceutical formulation.

In some embodiments:

(a) the first carrier component comprises from about 30% to about 50% by weight of the pharmaceutical formulation;

(b) the optional second carrier component, when present, comprises from about 30% to about 50% by weight of the pharmaceutical formulation;

(c) the emulsifying/solubilizing component comprises from about 1% to about 10% by weight of the pharmaceutical formulation;

(d) the optional anti-crystallization/solubilizing component, when present, comprises from about 1% to about 10% by weight of the pharmaceutical formulation; and

(e) the active pharmacological agent comprises from about 1% to about 25% by weight of the pharmaceutical formulation.

In some embodiments:

(a) the first carrier component comprises from about 65% to about 85% by weight of the pharmaceutical formulation;

(b) the optional second carrier component, when present, comprises up to about 30% by weight of the pharmaceutical formulation;

(c) the emulsifying/solubilizing component comprises from about 1% to about 10% by weight of the pharmaceutical formulation;

(d) the optional anti-crystallization/solubilizing component, when present, comprises from about 1% to about 10% by weight of the pharmaceutical formulation; and

(e) the active pharmacological agent comprises from about 1% to about 25% by weight of the pharmaceutical formulation.

In some embodiments:

(a) the first carrier component comprises from about 35% to about 45% by weight of the pharmaceutical formulation;

(b) the optional second carrier component, when present, comprises from about 35% to about 45% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 1% to about 25% by weight of the pharmaceutical formulation.

In some embodiments:

(a) the first carrier component comprises from about 50% to about 70% by weight of the pharmaceutical formulation;

(b) the optional second carrier component, when present, comprises up to about 30% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 1% to about 25% by weight of the pharmaceutical formulation.

In some embodiments:

(a) the first carrier component comprises from about 65% to about 85% by weight of the pharmaceutical formulation;

(b) the optional second carrier, when present, comprises up to about 10% by weight of the pharmaceutical formulation;

(c) the emulsifying/solubilizing component comprises from about 4% to about 6% by weight of the pharmaceutical formulation;

(d) the optional anti-crystallization/solubilizing component, when present, comprises from about 1% to about 15% by weight of the pharmaceutical formulation; and

(e) the active pharmacological agent comprises from about 1% to about 25% by weight of the pharmaceutical formulation.

In some embodiments:

(a) the first carrier component comprises from about 30% to about 50% by weight of the pharmaceutical formulation;

(b) the optional second carrier, when present, comprises from about 30% to about 50% by weight of the pharmaceutical formulation;

(c) the emulsifying/solubilizing component comprises from about 4% to about 6% by weight of the pharmaceutical formulation;

(d) the optional anti-crystallization/solubilizing component, when present, comprises from about 1% to about 15% by weight of the pharmaceutical formulation; and

(e) the active pharmacological agent comprises from about 1% to about 25% by weight of the pharmaceutical formulation.

In some embodiments:

(a) the first carrier component comprises from about 50% to about 70% by weight of the pharmaceutical formulation;

(b) the optional second carrier component, when present, comprises up to about 20% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 10% to about 20% by weight of the pharmaceutical formulation.

In some embodiments:

(a) the first carrier component comprises from about 30% to about 50% by weight of the pharmaceutical formulation;

(b) the optional second carrier component, when present, comprises from about 30% to about 50% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 10% to about 20% by weight of the pharmaceutical formulation.

In some embodiments:

(a) the first carrier component comprises from about 65% to about 75% by weight of the pharmaceutical formulation;

(b) the optional second carrier component, when present, comprises from about 5% to about 15% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 10% to about 20% by weight of the pharmaceutical formulation.

In some embodiments:

(a) the first carrier component comprises from about 75% to about 85% by weight of the pharmaceutical formulation;

(b) the optional second carrier component, when present, comprises from about 5% to about 15% by weight of the pharmaceutical formulation;

(c) the emulsifying/solubilizing component comprises from about 2% to about 7% by weight of the pharmaceutical formulation;

(d) the optional anti-crystallization/solubilizing component, when present, comprises from about 2% to about 7% by weight of the pharmaceutical formulation; and

(e) the active pharmacological agent comprises from about 10% to about 20% by weight of the pharmaceutical formulation.

In some of the embodiments disclosed herein, the emulsifying/solubilizing component is optional.

In some embodiments, the active pharmacological agent comprises the anhydrate crystal form (Form B) of 2-(3-fluoro-4-hydroxyphenyl)-7-vinyl-1,3-benzoxazol-5-ol. In some embodiments, the active pharmacological agent comprises at least about 50% by weight of 2-(3-fluoro-4-hydroxyphenyl)-7-vinyl-1,3-benzoxazol-5-ol which is present in the anhydrate crystal form (Form B): i.e., at least about 50% by weight of 2-(3-fluoro-4-hydroxyphenyl)-7-vinyl-1,3-benzoxazol-5-ol in the active pharmacological agent of the formulation is present as the particular anhydrate crystal form (Form B). In some embodiments, the active pharmacological agent comprises at least about 50%, at least about 60%, at least about 70%, at least about 80%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9%, by weight of the anhydrate crystal form (Form B) of 2-(3-fluoro-4-hydroxyphenyl)-7-vinyl-1,3-benzoxazol-5-ol. In some embodiments, the pharmaceutical formulations further comprises an additional active ingredient such as a progestin.

In some embodiments, the active pharmacological agent comprises from about 0.01% to about 80% by weight of the pharmaceutical formulation. In some embodiments, the active pharmacological agent comprises from about 0.01% to about 75% by weight of the pharmaceutical formulation. In some embodiments, the active pharmacological agent comprises from about 0.1% to about 50% by weight of the pharmaceutical formulation. In some embodiments, the active pharmacological agent comprises from about 0.1% to about 40% by weight of the pharmaceutical formulation. In some embodiments, the active pharmacological agent comprises from about 0.1% to about 30% by weight of the pharmaceutical formulation. In some embodiments, the active pharmacological agent comprises from about 0.1% to about 20% by weight of the pharmaceutical formulation. In some embodiments, the active pharmacological agent comprises from about 1% to about 40% by weight of the pharmaceutical formulation. In some embodiments, the active pharmacological agent comprises from about 1% to about 30% by weight of the pharmaceutical formulation. In some embodiments, the active pharmacological agent comprises from about 1% to about 25% by weight of the pharmaceutical formulation. In some embodiments, the active pharmacological agent comprises from about 1% to about 20% by weight of the pharmaceutical formulation. In some embodiments, the active pharmacological agent comprises from about 5% to about 25% by weight of the pharmaceutical formulation. In some embodiments, the active pharmacological agent comprises from about 10% to about 25% by weight of the pharmaceutical formulation. In some embodiments, the active pharmacological agent comprises from about 10% to about 20% by weight of the pharmaceutical formulation. In some embodiments, the active pharmacological agent comprises about 16.6% by weight of the pharmaceutical formulation. In some embodiments, the active pharmacological agent comprises about 15% by weight of the pharmaceutical formulation.

In some embodiments, the first carrier component comprises from about 10% to about 99.99% by weight of the pharmaceutical formulation. In some embodiments, the first carrier component comprises from about 10% to about 99% by weight of the pharmaceutical formulation. In some embodiments, the first carrier component comprises from about 20% to about 99% by weight of the pharmaceutical formulation. In some embodiments, the first carrier component comprises from about 30% to about 99% by weight of the pharmaceutical formulation. In some embodiments, the first carrier component comprises from about 30% to about 90% by weight of the pharmaceutical formulation. In some embodiments, the first carrier component comprises from about 50% to about 90% by weight of the pharmaceutical formulation. In some embodiments, the first carrier component comprises from about 50% to about 70% by weight of the pharmaceutical formulation. In some embodiments, the first carrier component comprises from about 30% to about 50% by weight of the pharmaceutical formulation. In some embodiments, the first carrier component comprises from about 35% to about 45% by weight of the pharmaceutical formulation. In some embodiments, the first carrier component comprises from about 65% to about 85% by weight of the pharmaceutical formulation. In some embodiments, the first carrier component comprises from about 65% to about 75% by weight of the pharmaceutical formulation. In some embodiments, the first carrier component comprises from about 75% to about 85% by weight of the pharmaceutical formulation.

In some embodiments, the first carrier component comprises about 15% by weight of the pharmaceutical formulation. In some embodiments, the first carrier component comprises about 18.33% by weight of the pharmaceutical formulation. In some embodiments, the first carrier component comprises about 35% by weight of the pharmaceutical formulation. In some embodiments, the first carrier component comprises about 38.33% by weight of the pharmaceutical formulation. In some embodiments, the first carrier component comprises about 40% by weight of the pharmaceutical formulation. In some embodiments, the first carrier component comprises about 60% by weight of the pharmaceutical formulation. In some embodiments, the first carrier component comprises about 70% by weight of the pharmaceutical formulation. In some embodiments, the first carrier component comprises about 75% by weight of the pharmaceutical formulation. In some embodiments, the first carrier component comprises about 78.33% by weight of the pharmaceutical formulation. In some embodiments, the first carrier component comprises about 81.5% by weight of the pharmaceutical formulation.

In some embodiments, the optional second carrier component, when present, comprises up to about 70% by weight of the pharmaceutical formulation. In some embodiments, the optional second carrier component, when present, comprises up to about 60% by weight of the pharmaceutical formulation. In some embodiments, the optional second carrier component, when present, comprises up to about 50% by weight of the pharmaceutical formulation. In some embodiments, the optional second carrier component, when present, comprises up to about 40% by weight of the pharmaceutical formulation. In some embodiments, the optional second carrier component, when present, comprises up to about 30% by weight of the pharmaceutical formulation. In some embodiments, the optional second carrier component, when present, comprises up to about 20% by weight of the pharmaceutical formulation. In some embodiments, the optional second carrier component, when present, comprises up to about 15% by weight of the pharmaceutical formulation. In some embodiments, the optional second carrier component, when present, comprises up to about 10% by weight of the pharmaceutical formulation. In some embodiments, the optional second carrier component, when present, comprises from about 10% to about 20% by weight of the pharmaceutical formulation. In some embodiments, the optional second carrier component, when present, comprises from about 30% to about 50% by weight of the pharmaceutical formulation. In some embodiments, the optional second carrier component, when present, comprises from about 35% to about 45% by weight of the pharmaceutical formulation. In some embodiments, the optional second carrier component, when present, comprises from about 5% to about 15% by weight of the pharmaceutical formulation.

In some embodiments, the optional second carrier component, when present, comprises about 8.33% by weight of the pharmaceutical formulation. In some embodiments, the optional second carrier component, when present, comprises about 15% by weight of the pharmaceutical formulation. In some embodiments, the optional second carrier component, when present, comprises about 18.33% by weight of the pharmaceutical formulation. In some embodiments, the optional second carrier component, when present, comprises about 35% by weight of the pharmaceutical formulation. In some embodiments, the optional second carrier component, when present, comprises about 38.33% by weight of the pharmaceutical formulation. In some embodiments, the optional second carrier component, when present, comprises about 40% by weight of the pharmaceutical formulation. In some embodiments, the optional second carrier component, when present, comprises about 60% by weight of the pharmaceutical formulation.

In some embodiments, the emulsifiying/solubilizing component is optional. In some embodiments, the emulsifiying/solubilizing component is present. All of the embodiments in this paragraph can be provided for the liquid or semi-solid pharmaceutical formulations of the invention where the emulsifying/solubilizing component is present, or for liquid or semi-solid pharmaceutical formulations of the invention where the emulsifying/solubilizing component is optional. In some embodiments, the emulsifying/solubilizing component comprises from about 0.01% to about 30% by weight of the pharmaceutical formulation. In some embodiments, the emulsifying/solubilizing component comprises from about 0.01% to about 20% by weight of the pharmaceutical formulation. In some embodiments, the emulsifying/solubilizing component comprises from about 0.1% to about 20% by weight of the pharmaceutical formulation. In some embodiments, the emulsifying/solubilizing component comprises from about 0.1% to about 15% by weight of the pharmaceutical formulation. In some embodiments, the emulsifying/solubilizing component comprises from about 0.1% to about 10% by weight of the pharmaceutical formulation. In some embodiments, the emulsifying/solubilizing component comprises from about 1% to about 10% by weight of the pharmaceutical formulation. In some embodiments, the emulsifying/solubilizing component comprises from about 1% to about 8% by weight of the pharmaceutical formulation. In some embodiments, the emulsifying/solubilizing component comprises from about 2% to about 7% by weight of the pharmaceutical formulation. In some embodiments, the emulsifying/solubilizing component comprises from about 4% to about 6% by weight of the pharmaceutical formulation. In some embodiments, the emulsifying/solubilizing component comprises about 1% by weight of the pharmaceutical formulation. In some embodiments, the emulsifying/solubilizing component comprises about 5% by weight of the pharmaceutical formulation.

In some embodiments, the optional anti-crystallization/solubilizing component, when present, comprises from about 0.01% to about 30% by weight of the pharmaceutical formulation. In some embodiments, the optional anti-crystallization/solubilizing component, when present, comprises from about 0.01% to about 10% by weight of the pharmaceutical formulation. In some embodiments, the optional anti-crystallization/solubilizing component, when present, comprises from about 0.1% to about 20% by weight of the pharmaceutical formulation. In some embodiments, the optional anti-crystallization/solubilizing component, when present, comprises from about 0.1% to about 15% by weight of the pharmaceutical formulation. In some embodiments, the optional anti-crystallization/solubilizing component, when present, comprises from about 0.1% to about 10% by weight of the pharmaceutical formulation. In some embodiments, the optional anti-crystallization/solubilizing component, when present, comprises from about 1% to about 20% by weight of the pharmaceutical formulation. In some embodiments, the optional anti-crystallization/solubilizing component, when present, comprises from about 1% to about 15% by weight of the pharmaceutical formulation. In some embodiments, the optional anti-crystallization/solubilizing component, when present, comprises from about 1% to about 10% by weight of the pharmaceutical formulation. In some embodiments, the optional anti-crystallization/solubilizing component, when present, comprises from about 1% to about 8% by weight of the pharmaceutical formulation. In some embodiments, the optional anti-crystallization/solubilizing component, when present, comprises from about 2% to about 7% by weight of the pharmaceutical formulation. In some embodiments, the optional anti-crystallization/solubilizing component, when present, comprises about 10% by weight of the pharmaceutical formulation. In some embodiments, the optional anti-crystallization/solubilizing component, when present, comprises about 5% by weight of the pharmaceutical formulation.

In some embodiments, the liquid or semi-solid pharmaceutical formulation comprises from about 1 mg to about 200 mg of active pharmacological agent. In some embodiments, the liquid or semi-solid pharmaceutical formulation comprises from about 1 mg to about 10 mg of active pharmacological agent. In some embodiments, the liquid or semi-solid pharmaceutical formulation comprises from about 10 mg to about 50 mg of active pharmacological agent. In some embodiments, the liquid or semi-solid pharmaceutical formulation comprises from about 50 mg to about 100 mg of active pharmacological agent. In some embodiments, the liquid or semi-solid pharmaceutical formulation comprises from about 100 mg to about 200 mg of active pharmacological agent.

In some embodiments, each of the pharmaceutical formulations disclosed herein is a semi-solid pharmaceutical formulation. In some embodiments, each of the pharmaceutical formulations disclosed herein is not a liquid formulation. In some embodiments, each of the pharmaceutical formulations disclosed herein is a semi-solid pharmaceutical formulation and each carrier component is a semi-solid substance.

In some embodiments, when the optional emulsifying/solubilizing component is not present, the optional anti-crystallization/solubilizing component or the optional second carrier component is present; and when the optional anti-crystallization/solubilizing component is not present, the optional emulsifying/solubilizing component or the optional second carrier component is present.

In some embodiments, when the optional emulsifying/solubilizing component is not present, the optional anti-crystallization/solubilizing component is present.

In some embodiments, when the optional emulsifying/solubilizing component is not present, the optional second carrier component is present.

In some embodiments, when the optional anti-crystallization/solubilizing component is not present, the optional emulsifying/solubilizing component is present.

In some embodiments, when the optional anti-crystallization/solubilizing component is not present, the optional second semi-solid component is present.

In some embodiments, each optional component is present in the formulation.

In some embodiments, each component comprises only one material.

In some embodiments, the optional emulsifying/solubilizing component is present. In some embodiments, the emulsifying/solubilizing component is optional.

In some embodiments, the liquid or semi-solid pharmaceutical formulations described herein do not comprise a disintegrant.

In some embodiments, the liquid or semi-solid pharmaceutical formulations described herein do not comprise a disintegrant, wherein the disintegrant comprises one or more of cellulose floc, modified cellulose, starch, sodium starch glycolate, pregelatinized starch, dibasic calcium phosphate, magnesium carbonate, magnesium oxide, calcium silicate, silicon dioxide, silicon dioxide aerogel, silica, clay, veegum, xanthan gum, talc, croscarmellose sodium, crosprovidone, stearate, alginic acid, sodium alginate, ion exchange resin, or effervescent system based on food acids and an alkaline carbonate component.

In some embodiments, the liquid or semi-solid pharmaceutical formulations described herein do not comprise one or more of cellulose floc, modified cellulose, starch, sodium starch glycolate, pregelatinized starch, dibasic calcium phosphate, magnesium carbonate, magnesium oxide, calcium silicate, silicon dioxide, silicon dioxide aerogel, silica, clay, veegum, xanthan gum, talc, croscarmellose sodium, crosprovidone, stearate, alginic acid, sodium alginate, ion exchange resin, or effervescent system based on food acids and an alkaline carbonate component.

In some embodiments, when the liquid or semi-solid pharmaceutical formulations described herein comprises one or more ingredients selected from cellulose floc, modified cellulose, starch, sodium starch glycolate, pregelatinized starch, dibasic calcium phosphate, magnesium carbonate, magnesium oxide, calcium silicate, silicon dioxide, silicon dioxide aerogel, silica, clay, veegum, xanthan gum, talc, croscarmellose sodium, crosprovidone, stearate, alginic acid, sodium alginate, ion exchange resin, and effervescent system based on food acids and an alkaline carbonate component, then the sum of the ingredients is not in the range of about 0.01% to about 10% by weight of the pharmaceutical formulation.

In some embodiments, the liquid or semi-solid pharmaceutical formulations described herein do not comprise about 0.01% to about 10% of a disintegrant by weight of the pharmaceutical formulation.

In some embodiments, the liquid or semi-solid pharmaceutical formulations described herein do not comprise about 0.01 to about 10% of a disintegrant by weight of the pharmaceutical formulation, wherein the disintegrant comprises one or more of cellulose floc, modified cellulose, starch, sodium starch glycolate, pregelatinized starch, dibasic calcium phosphate, magnesium carbonate, magnesium oxide, calcium silicate, silicon dioxide, silicon dioxide aerogel, silica, clay, veegum, xanthan gum, talc, croscarmellose sodium, crosprovidone, stearate, alginic acid, sodium alginate, ion exchange resin, or effervescent system based on food acids and an alkaline carbonate component.

In some embodiments, the first carrier component is not sorbitol. In some embodiments, the optional second carrier component is not sorbitol. In some embodiments, the pharmaceutical formulations disclosed herein do not comprise water. In some embodiments, the pharmaceutical formulations disclosed herein do not comprise benzyl alcohol. In some embodiments, the pharmaceutical formulations disclosed herein do not comprise sorbic acid.

In some embodiments, the first carrier component, the optional second carrier component, the emulsifying/solubilizing component, and the optional anti-crystallization/solubilizing component are each different materials.

As used herein, the term “carrier component” refers to one or more substances that can be used to solubilize, dissolve, emulsify, and/or suspend the active pharmacological agent in the liquid or semi-solid pharmaceutical formulation. The first carrier component have a number of additional functions, besides providing a carrier medium for the active pharmacological agent. For example, in some embodiments, the first carrier component comprises at least one substance that enhances bioavailability of the active pharmacological agent. In some embodiments, the first carrier component comprises at least one substance that improves dissolution of the active pharmacological agent. In some embodiments, the first carrier component comprises at least one substance that improves the stability of the pharmacological formulation.

In some embodiments, the first carrier is a substance suitable for forming a liquid or semi-solid pharmaceutical formulation. In some embodiments, the first carrier comprises at least one semi-solid substance. In some embodiments, the first carrier comprises at least one liquid substance. In some embodiments, the first carrier component comprises at least one semi-solid substance. In some embodiments, the first carrier component comprises at least one lipid substance. In some embodiments, the first carrier component comprises at least one surfactant. In some embodiments, the first carrier component comprises a mixture of at least one lipid substance and at least one surfactant. In some embodiments, the first carrier component comprises at least one substance that is water-soluble. In some embodiments, the first carrier component comprises at least one substance that forms vesicles in water. In some embodiments, the first carrier component comprises at least one substance that forms micelles in water. Non-limiting examples of suitable carrier components can be found in Remington's Pharmaceutical Sciences, 17th ed., Mack Publishing Company, Easton, Pa., 1985, which is hereby incorporated by reference in its entirety.

In some embodiments, the first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil.

In some embodiments, the first carrier component comprises one or more of lauroyl macrogol glycerides, caprylocaproyl macrogolglycerides, stearoyl macrogol glycerides, linoleoyl macrogol glycerides, oleoyl macrogol glycerides, polyethylene glycol, polyoxyethylene fatty alcohol ether, polyethoxylated fatty acid ester, polyoxyethylene-glycerol fatty ester, polyglycolized glycerides, polyethoxylated sorbitan ester, polyethoxylated castor oil, or polyethoxylated vegetable oil.

In some embodiments, the first carrier component comprises one or more of lauroyl macrogol glycerides, caprylocaproyl macrogolglycerides, or polyethylene glycol.

In some embodiments, the first carrier component comprises caprylocaproyl macrogolglycerides.

In some embodiments, the first carrier component comprises lauroyl macrogol glycerides.

In some embodiments of the invention, it may be desirable to add an optional second carrier component. The optional second carrier component have a number of possible functions, in addition to providing a carrier medium for solubilization, dissolution, emulsification, or suspension of the active pharmacological agent. For example, in some embodiments the optional second liquid or sem-solid carrier component comprises at least one substance that lowers the viscosity of the pharmaceutical formulation. In some embodiments, the optional second carrier component comprises at least one substance that enhances bioavailability of the active pharmacological agent. In some embodiments, the optional second carrier component comprises at least one substance that improves dissolution of the active pharmacological agent. In some embodiments, the optional second carrier component comprises at least one substance that improves the stability of the pharmacological formulation.

In some embodiments, the optional second carrier comprises at least one semi-solid substance. In some embodiments, the optional second carrier is a substance suitable for forming a liquid or semi-solid pharmaceutical formulation. In some embodiments, the optional second carrier comprises at least one liquid substance. In some embodiments, the second carrier component comprises at least one semi-solid substance. In some embodiments, the optional second carrier component comprises at least one lipid substance. In some embodiments, the optional second carrier component comprises at least one surfactant. In some embodiments, the optional second carrier component comprises a mixture of at least one lipid substance and at least one surfactant. In some embodiments, the optional second carrier component comprises at least one substance that is water-soluble. In some embodiments, the optional second carrier component comprises at least one substance that forms vesicles in water. In some embodiments, the optional second carrier component comprises at least one substance that forms micelles in water.

In some embodiments, the optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil.

In some embodiments, the optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyethylene glycol, polyoxyethylene fatty alcohol ether, polyethoxylated fatty acid ester, polyoxyethylene-glycerol fatty ester, polyglycolized glycerides, polyethoxylated sorbitan ester, polyethoxylated castor oil, or polyethoxylated vegetable oil.

In some embodiments, the optional second carrier component, when present, comprises lauroyl macrogol glycerides or caprylocaproyl macrogolglycerides.

In some embodiments, the optional second carrier component, when present, comprises lauroyl macrogol glycerides.

In some embodiments, the optional second carrier component, when present, comprises caprylocaproyl macrogolglycerides.

As used herein, the term “emulsifying/solubilizing component” refers, in one aspect, to a substance that improves the emulsification, or suspension of the active pharmacological agent in the pharmaceutical formulation. As used herein, the term “emulsifying/solubilizing component” refers, in an alternate aspect or additional aspect, to a substance that improves the stability of the pharmaceutical formulation and/or the compatibility of the components in the formulation. As used herein, the term “emulsifying/solubilizing component” refers, in an additional or alternative aspect, to a substance that improves bioavailability or dissolution of the active pharmacological agent during administration. In some embodiments, the emulsifying/solubilizing component comprises at least one substance that improves the homogeneity of the pharmaceutical formulations of the invention. In some embodiments, the emulsifying/solubilizing component comprises at least one substance that improves the rheology of the pharmaceutical formulations of the invention.

In some embodiments, the optional emulsifying/solubilizing component comprises at least one surfactant or emulsifying agent. As used herein, the term “emulsifying agent” refers to a substance that can emulsify a substance in water or in oil. For example, suitable emulsifying agents include, but are not limited to oil-in-water emulsifiers, as well as wetting agents and water-in-oil emulsifiers. In some embodiments, the emulsifying/solubilizing component comprises at least one oil-in-water emulsifying agent. In some embodiments, the emulsifying/solubilizing component comprises at least one water-in-oil emulsifier. In some embodiments, the emulsifying/solubilizing component comprises at least one surfactant. In some embodiments, the emulsifying/solubilizing agent comprises at least one substance with a hydrophile-lipophile balance (HLB) from about 4 to about 7. In some embodiments, the emulsifying/solubilizing agent comprises at least one substance with a hydrophile-lipophile balance (HLB) from about 7 to about 9. In some embodiments, the emulsifying/solubilizing agent comprises at least one substance with a hydrophile-lipophile balance (HLB) from about 8 to about 18. In some embodiments, the emulsifying/solubilizing agent comprises at least one substance with a hydrophile-lipophile balance (HLB) from about 10 to about 18. In some embodiments, the emulsifying/solubilizing agent comprises at least one substance with a hydrophile-lipophile balance (HLB) from about 13 to about 18. In some embodiments, the emulsifying/solubilizing agent comprises at least one substance with a hydrophile-lipophile balance (HLB) from about 14 to about 16.

In some embodiments, the emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

In some embodiments, the emulsifying/solubilizing component comprises one or more of metallic alkyl sulfate, salts of fatty acids, lauroyl macrogol glycerides, caprylocaproyl macrogolglycerides, stearoyl macrogol glycerides, linoleoyl macrogol glycerides, oleoyl macrogol glycerides, polyethylene glycol, polyoxyethylene-polyoxypropylene copolymer, polyoxyethylene fatty alcohol ether, polyethoxylated fatty acid ester, polyoxyethylene-glycerol fatty ester, polyglycolized glycerides, polyglycerol fatty acid ester, polyethoxylated sorbitan ester, polyethoxylated castor oil, or polyethoxylated vegetable oil.

In some embodiments, the emulsifying/solubilizing component comprises one or more of metallic alkyl sulfate, salts of fatty acids, polyoxyethylene-polyoxypropylene copolymer, polyoxyethylene fatty alcohol ether, polyethoxylated fatty acid ester, polyoxyethylene-glycerol fatty ester, polyethoxylated sorbitan ester, or polyethoxylated castor oil.

In some embodiments, the emulsifying/solubilizing component comprises polyethoxylated sorbitan ester.

In some embodiments, the emulsifying/solubilizing component comprises polyoxyethylene-20 sorbitan monolaurate, polyoxyethylene-4 sorbitan monolaurate, polyoxyethylene-20 sorbitan monopalmitate, polyoxyethylene-20 sorbitan monostearate, polyoxyethylene-20 sorbitan monostearate, polyoxyethylene-4 sorbitan monostearate, polyoxyethylene-20 sorbitan tristearate, polyoxyethylene-20 sorbitan monooleate, polyoxyethylene-20 sorbitan monooleate, polyoxyethylene-5 sorbitan monooleate, or polyoxyethylene-20 sorbitan trioleate.

In some embodiments, the emulsifying/solubilizing component comprises polyoxyethylene-20 sorbitan monooleate.

The embodiments described herein for the emulsifying/solubilizing component can also be provided for the liquid or semi-solid formulations wherein emulsifying/solubilizing component is optional.

As used herein, the term “anti-crystallization/solubilizing component” refers, in one aspect, to a substance that lowers the tendency of the active pharmacological agent to convert to another crystal/crystalline form or the amorphous form during processing or storage. As used herein, the term “anti-crystallization/solubilizing component” refers, in an additional or alternative aspect, to a substance that improves bioavailability or dissolution of the active pharmacological agent during administration. As used herein, the term “anti-crystallization/solubilizing component” refers, in an additional or alternative aspect, to a substance that improves the emulsification, or suspension of the active pharmacological agent in the pharmaceutical formulation. In some embodiments, the optional anti-crystallization/solubilizing agent comprises at least one a water-soluble substance. In some embodiments, the optional anti-crystallization/solubilizing agent comprises at least one hydrophilic substance. In some embodiments, the optional anti-crystallization/solubilizing agent comprises at least one surfactant.

In some embodiments, the optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

In some embodiments, the optional anti-crystallization/solubilizing component, when present, comprises one or more of polyvinylpyrrolidone, lauroyl macrogol glycerides, caprylocaproyl macrogolglycerides, stearoyl macrogol glycerides, linoleoyl macrogol glycerides, oleoyl macrogol glycerides, polyoxyethylene-polyoxypropylene copolymer, polyoxyethylene fatty alcohol ether, polyethoxylated fatty acid ester, polyoxyethylene-glycerol fatty ester, polyethoxylated sorbitan ester, or polyethoxylated castor oil.

In some embodiments, the optional anti-crystallization/solubilizing component, when present, comprises polyvinylpyrrolidone.

In some embodiments, the optional anti-crystallization/solubilizing component, when present, comprises povidone K12, K17, K25, K30, K60, K90, or K120.

In some embodiments, the optional anti-crystallization/solubilizing component, when present, comprises povidone K25.

In some embodiments:

(a) the first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil;

(b) the optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil;

(c) the emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil; and

(d) the optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

In some embodiments:

(a) the first carrier component comprises one or more of lauroyl macrogol glycerides, caprylocaproyl macrogolglycerides, or polyethylene glycol;

(b) the optional carrier component, when present, comprises lauroyl macrogol glycerides or caprylocaproyl macrogolglycerides;

(c) the emulsifying/solubilizing component comprises polyethoxylated sorbitan ester; and

(d) the optional anti-crystallization/solubilizing component, when present, comprises polyvinylpyrrolidone.

In some embodiments:

(a) the first carrier component comprises lauroyl macrogol glycerides;

(b) the optional second carrier component, when present, comprises caprylocaproyl macrogolglycerides;

(c) the emulsifying/solubilizing component comprises polyoxyethylene-20 sorbitan monooleate; and

(d) the optional anti-crystallization/solubilizing component, when present, comprises polyvinylpyrrolidone.

The embodiments described herein can also be provided for the liquid or semi-solid formulations wherein emulsifying/solubilizing component is optional.

The present invention further provides a process for preparing the liquid or semi-solid pharmaceutical formulations of the invention comprising mixing the first carrier component and the active pharmaceutical agent with sufficient heating to obtain a suspension or solution of the active pharmaceutical agent.

In some embodiments, the mixing is performed in a heated jacketed bowl.

In some embodiments, the first carrier is melted prior to the mixing.

In some embodiments, the process further comprises mixing the first carrier component, the second optional carrier component, if present, the emulsifying/solubilizing component and the optional anti-crystallization/solubilizing component, if present, with sufficient heating to enable blending, prior to the mixing to form the suspension or solution.

In some embodiments, the process further comprises melting the optional second carrier component, the emulsifying/solubilizing component, and the optional anti-crystallization/solubilizing component prior to the mixing of the first carrier component, the optional second carrier component, the emulsifying/solubilizing component, and the optional anti-crystallization/solubilizing component.

In some embodiments, the process further comprises adding the optional second carrier component, the emulsifying/solubilizing component, and the optional anti-crystallization/solubilizing component in separate stages to the first carrier component.

The processes described herein can be used to prepare any of the liquid or semi-solid pharmaceutical formulations described herein, as well as any combination and subcombinations of the embodiments thereof.

In some embodiments:

(a) the first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil;

(b) the optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil;

(c) the emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil; and

(d) the optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

In some embodiments:

(a) the first carrier component comprises one or more of lauroyl macrogol glycerides, caprylocaproyl macrogolglycerides, or polyethylene glycol;

(b) the optional second carrier component, when present, comprises lauroyl macrogol glycerides or caprylocaproyl macrogolglycerides;

(c) the emulsifying/solubilizing component comprises polyethoxylated sorbitan ester; and

(d) the optional anti-crystallization/solubilizing component, when present, comprises polyvinylpyrrolidone.

In some embodiments:

(a) the first carrier component comprises caprylocaproyl macrogolglycerides;

(b) the optional second carrier component, when present, comprises lauroyl macrogol glycerides;

(c) the emulsifying/solubilizing component comprises polyoxyethylene-20 sorbitan monooleate; and

(d) the optional anti-crystallization/solubilizing component, when present, comprises polyvinylpyrrolidone.

In some embodiments:

(a) the first carrier component comprises lauroyl macrogol glycerides;

(b) the optional second carrier component, when present, comprises caprylocaproyl macrogolglycerides;

(c) the emulsifying/solubilizing component comprises polyoxyethylene-20 sorbitan monooleate; and

(d) the optional anti-crystallization/solubilizing component, when present, comprises polyvinylpyrrolidone.

The embodiments of the processes described herein can also be provided for liquid or semi-solid pharmaceutical formulations wherein the emulsifying/solubilizing component is optional.

The present invention further provides a product of the process for preparing the liquid or semi-solid pharmaceutical formulations of the invention.

The present invention further provides hard gel or soft gel capsules comprising the liquid or semi-solid pharmaceutical formulations of the invention. Any of the liquid or semi-solid pharmaceutical formulations described herein, as well as any combination and subcombinations of the embodiments thereof, can be used to prepare the capsules of the invention.

As used herein, a “semi-solid” substance refers to a substance having the attributes of both a solid and a liquid. Examples of semi-solids include creams, gels, oilments, emulsions and suspensions. As used herein, the liquid formulations of the present invention include those stable liquid suspensions and emulsions comprising both liquid and solid components [wherein the solid component includes the anhydrate crystal form (Form B) of 2-(3-fluoro-4-hydroxyphenyl)-7-vinyl-1,3-benzoxazol-5-ol that is not dissolved in the liquid component]. In some embodiments, the anhydrate crystal form (Form B) of 2-(3-fluoro-4-hydroxyphenyl)-7-vinyl-1,3-benzoxazol-5-ol in the liquid formulations of the present invention remains substantially intact during processing, transportation, and/or storage under ambient conditions for a period greater than about 36 hours, greater than about 1 week, greater than about 1 month, greater than about 6 months, or greater than about 1 year. For example, in such stable suspensions, the solid stays homogeneous in the suspension (i.e., the solid does not precipitate out) during storage under ambient conditions for a period greater than about 36 hours, greater than about 1 week, greater than about 1 month, greater than about 6 months, or greater than about 1 year.

In some embodiments, the liquid or semi-solid pharmaceutical formulation of the present invention is stabilized and the anhydrate crystal form (Form B) of 2-(3-fluoro-4-hydroxyphenyl)-7-vinyl-1,3-benzoxazol-5-ol stays homogeneous in the formulation (i.e., the crystal forms do not separate from other components in the formulation). In some embodiments, the anhydrate crystal form stays homogeneous in the formulation during storage under ambient conditions for a period greater than about 36 hours, greater than about 1 week, greater than about 1 month, greater than about 6 months, or greater than about 1 year.

The anhydrate crystal form (Form B) of 2-(3-fluoro-4-hydroxyphenyl)-7-vinyl-1,3-benzoxazol-5-ol which is present in the liquid or semi-solid formulation remains substantially intact during processing, transportation, and/or storage. In some embodiments, the anhydrate crystal form (Form B) of 2-(3-fluoro-4-hydroxyphenyl)-7-vinyl-1,3-benzoxazol-5-ol which is present in the liquid or semi-solid formulation remains substantially intact (i.e., no conversion to another crystal form or an amorphous form) during processing, storage and/or transportation under ambient conditions for a period greater than about 36 hours, greater than about 1 week, greater than about 1 month, greater than about 6 months, or greater than about 1 year.

### Solid Pharmaceutical Formulations

In another aspect, the present invention also provides a solid pharmaceutical formulation comprising:

(a) a first diluent/filler component comprising from about 30% to about 95% by weight of the formulation;

(b) an optional second diluent/filler component, when present, comprising up to about 40% by weight of the pharmaceutical formulation;

(c) a disintegrant component comprising from about 0.01% to about 30% by weight of the pharmaceutical formulation;

(d) a binder component comprising from about 0.01% to about 20% by weight of the pharmaceutical formulation;

(e) a wetting agent component comprising from about 0.01% to about 20% by weight of the pharmaceutical formulation;

(f) an optional lubricant component, when present, comprising from about 0.01% to about 10% by weight of the pharmaceutical formulation; and

(g) an active pharmacological agent comprising from about 0.01% to about 80% by weight of the pharmaceutical formulation, wherein the active pharmacological agent comprises the anhydrate crystal form (Form B) of 2-(3-fluoro-4-hydroxyphenyl)-7-vinyl-1,3-benzoxazol-5-ol. These pharmaceutical formulations will be referred to herein as the “solid formulations” to distinguish them from the liquid or semi-solid formulations disclosed herein.

In some embodiments of the solid formulations:

(a) the first diluent/filler component comprises from about 40% to about 80% by weight of the formulation;

(b) the optional second diluent/filler component, when present, comprises up about 20% by weight of the pharmaceutical formulation;

(c) the disintegrant component comprises from about 0.1% to about 20% by weight of the pharmaceutical formulation;

(d) the binder component comprises from about 0.1% to about 10% by weight of the pharmaceutical formulation;

(e) the wetting agent component comprises from about 0.1% to about 10% by weight of the pharmaceutical formulation; and

(f) the optional lubricant component, when present, comprises from about 0.01% to about 5% by weight of the pharmaceutical formulation; and

(g) the active pharmacological agent comprises from about 0.1% to about 50% by weight of the pharmaceutical formulation.

In some embodiments of the solid formulations:

(a) the first diluent/filler component comprises from about 40% to about 80% by weight of the pharmaceutical formulation;

(b) the optional second diluent/filler component, when present, comprises from about 10% to about 20% by weight of the pharmaceutical formulation;

(c) the disintegrant component comprises from about 1% to about 10% by weight of the pharmaceutical formulation;

(d) the binder component comprises from about 1% to about 8% by weight of the pharmaceutical formulation;

(e) the wetting agent component comprises from 1% to about 8% by weight of the pharmaceutical formulation;

(f) the optional lubricant component, when present, comprises from about 0.1% to about 2% by weight of the pharmaceutical formulation; and

(g) the active pharmacological agent comprises from about 1% to about 40% by weight of the pharmaceutical formulation.

In some embodiments of the solid formulations:

(a) the first diluent/filler component comprises from about 60% to about 80% by weight of the pharmaceutical formulation;

(b) the optional second diluent/filler component, when present, comprises from about 10% to about 20% by weight of the pharmaceutical formulation;

(c) the disintegrant component comprises from about 2% to about 6% by weight of the pharmaceutical formulation;

(d) the binder component comprises from about 1% to about 3% by weight of the pharmaceutical formulation;

(e) the wetting agent component comprises from about 1% to about 3% by weight of the pharmaceutical formulation;

(f) the optional lubricant component, when present, comprises from about 0.1% to about 1% by weight of the pharmaceutical formulation; and

(g) the active pharmacological agent comprises from about 1% to about 10% by weight of the pharmaceutical formulation.

In some embodiments of the solid formulations:

(a) the first diluent/filler component comprises from about 40% to about 60% by weight of the pharmaceutical formulation;

(b) the optional second diluent/filler component, when present, comprises from about 10% to about 20% by weight of the pharmaceutical formulation;

(c) the disintegrant component comprises from about 2% to about 6% by weight of the pharmaceutical formulation;

(d) the binder component comprises from about 1% to about 3% by weight of the pharmaceutical formulation;

(e) the wetting agent component comprises from about 1% to about 3% by weight of the pharmaceutical formulation;

(f) the optional lubricant component, when present, comprises from about 0.1% to about 1% by weight of the pharmaceutical formulation; and

(g) the active pharmacological agent comprises from about 10% to about 30% by weight of the pharmaceutical formulation.

In some embodiments of the solid formulations:

(a) the first diluent/filler component comprises from about 38% to about 95% by weight of the formulation;

(b) the optional second diluent/filler component comprises from about 5% to about 25% by weight of the pharmaceutical formulation;

(c) the disintegrant component comprises from about 0.5% to about 20% by weight of the pharmaceutical formulation;

(d) the binder component comprises from about 0.5% to about 10% by weight of the pharmaceutical formulation;

(e) the wetting agent component comprises from about 0.5% to about 8% by weight of the pharmaceutical formulation; and

(f) the optional lubricant component, when present, comprises from about 0.01% to about 5% by weight of the pharmaceutical formulation; and

(g) the active pharmacological agent comprises from about 0.01% to about 75% by weight of the pharmaceutical formulation.

In some embodiments of the solid formulations:

(a) the first diluent/filler component comprises from about 38% to about 95% by weight of the formulation;

(b) the optional second diluent/filler component comprises from about 5% to about 25% by weight of the pharmaceutical formulation;

(c) the disintegrant component comprises from about 0.5% to about 20% by weight of the pharmaceutical formulation;

(d) the binder component comprises from about 0.5% to about 5% by weight of the pharmaceutical formulation;

(e) the wetting agent component comprises from about 1.3% to about 5% by weight of the pharmaceutical formulation;

(f) the optional lubricant component, when present, comprises from about 0.01% to about 5% by weight of the pharmaceutical formulation; and

(g) the active pharmacological agent comprises from about 0.01% to about 75% by weight of the pharmaceutical formulation.

In some embodiments of the solid formulations:

(a) the first diluent/filler component comprises from about 38% to about 95% by weight of the formulation;

(b) the optional second diluent/filler component comprises from about 5% to about 25% by weight of the pharmaceutical formulation;

(c) the disintegrant component comprises from about 0.5% to about 20% by weight of the pharmaceutical formulation;

(d) the binder component comprises from about 1% to about 3% by weight of the pharmaceutical formulation;

(e) the wetting agent component comprises from about 1.3% to about 4% by weight of the pharmaceutical formulation; and

(f) the optional lubricant component, when present, comprises from about 0.01% to about 5% by weight of the pharmaceutical formulation; and

(g) the active pharmacological agent comprises from about 0.01% to about 75% by weight of the pharmaceutical formulation.

In some embodiments of the solid formulations:

(a) the first diluent/filler component comprises from about 40% to about 80% by weight of the pharmaceutical formulation;

(b) the optional second diluent/filler component, when present, comprises from about 5% to about 20% by weight of the pharmaceutical formulation;

(c) the disintegrant component comprises from about 0.5% to about 10% by weight of the pharmaceutical formulation;

(d) the binder component comprises from about 0.5% to about 10% by weight of the pharmaceutical formulation;

(e) the wetting agent component comprises from 0.5% to about 10% by weight of the pharmaceutical formulation; and

(f) the optional lubricant component, when present, comprises from about 0.1% to about 5% by weight of the pharmaceutical formulation; and

(g) the active pharmacological agent comprises from about 0.1% to about 50% by weight of the pharmaceutical formulation.

In some embodiments of the solid formulations:

(a) the first diluent/filler component comprises from about 40% to about 80% by weight of the pharmaceutical formulation;

(b) the optional second diluent/filler component, when present, comprises from about 5% to about 20% by weight of the pharmaceutical formulation;

(c) the disintegrant component comprises from about 3% to about 5% by weight of the pharmaceutical formulation;

(d) the binder component comprises from about 1% to about 3% by weight of the pharmaceutical formulation;

(e) the wetting agent component comprises from 1% to about 3% by weight of the pharmaceutical formulation;

(f) the optional lubricant component, when present, comprises from about 0.1% to about 2% by weight of the pharmaceutical formulation; and

(g) the active pharmacological agent comprises from about 1% to about 35% by weight of the pharmaceutical formulation.

In some embodiments of the solid formulations:

(a) the first diluent/filler component comprises from about 40% to about 80% by weight of the pharmaceutical formulation;

(b) the optional second diluent/filler component, when present, comprises from about 10% to about 20% by weight of the pharmaceutical formulation;

(c) the disintegrant component comprises from about 1% to about 7% by weight of the pharmaceutical formulation;

(d) the binder component comprises from about 1% to about 5% by weight of the pharmaceutical formulation;

(e) the wetting agent component comprises from 1.3% to about 5% by weight of the pharmaceutical formulation;

(f) the optional lubricant component, when present, comprises from about 0.1% to about 2% by weight of the pharmaceutical formulation; and

(g) the active pharmacological agent comprises from about 0.1% to about 50% by weight of the pharmaceutical formulation.

In some embodiments of the solid formulations:

(a) the first diluent/filler component comprises from about 40% to about 80% by weight of the pharmaceutical formulation;

(b) the optional second diluent/filler component, when present, comprises from about 10% to about 20% by weight of the pharmaceutical formulation;

(c) the disintegrant component comprises from about 3% to about 5% by weight of the pharmaceutical formulation;

(d) the binder component comprises from about 1% to about 3% by weight of the pharmaceutical formulation;

(e) the wetting agent component comprises from 1.5% to about 4% by weight of the pharmaceutical formulation;

(f) the optional lubricant component, when present, comprises from about 0.1% to about 1% by weight of the pharmaceutical formulation; and

(g) the active pharmacological agent comprises from about 0.1% to about 40% by weight of the pharmaceutical formulation.

In some embodiments of the solid formulations:

(a) the first diluent/filler component comprises from about 60% to about 80% by weight of the pharmaceutical formulation;

(b) the optional second diluent/filler component, when present, comprises from about 10% to about 20% by weight of the pharmaceutical formulation;

(c) the disintegrant component comprises about 4% by weight of the pharmaceutical formulation;

(d) the binder component comprises about 2% by weight of the pharmaceutical formulation;

(e) the wetting agent component comprises about 2% by weight of the pharmaceutical formulation;

(f) the optional lubricant component, when present, comprises from about 0.1% to about 1% by weight of the pharmaceutical formulation; and

(g) the active pharmacological agent comprises from about 1% to about 10% by weight of the pharmaceutical formulation.

In some embodiments of the solid formulations:

(a) the first diluent/filler component comprises from about 40% to about 60% by weight of the pharmaceutical formulation;

(b) the optional second diluent/filler component, when present, comprises from about 10% to about 20% by weight of the pharmaceutical formulation;

(c) the disintegrant component comprises about 4% by weight of the pharmaceutical formulation;

(d) the binder component comprises about 2% by weight of the pharmaceutical formulation;

(e) the wetting agent component comprises about 2% by weight of the pharmaceutical formulation;

(f) the optional lubricant component, when present, comprises from about 0.1% to about 1% by weight of the pharmaceutical formulation; and

(g) the active pharmacological agent comprises from about 10% to about 30% by weight of the pharmaceutical formulation.

In some embodiments of the solid formulations, the active pharmacological agent comprises the anhydrate crystal form (Form B) of 2-(3-fluoro-4-hydroxyphenyl)-7-vinyl-1,3-benzoxazol-5-ol. In some embodiments of the solid formulations, the active pharmacological agent comprises at least about 50% by weight of the anhydrate crystal form (Form B) of 2-(3-fluoro-4-hydroxyphenyl)-7-vinyl-1,3-benzoxazol-5-ol:): i.e., at least about 50% by weight of 2-(3-fluoro-4-hydroxyphenyl)-7-vinyl-1,3-benzoxazol-5-ol in the active pharmacological agent of the formulation is present as the particular anhydrate crystal form (Form B). In some embodiments of the solid formulations, the active pharmacological agent comprises at least about 50%, at least about 60%, at least about 70%, at least about 80%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, or at least about 99.9%, by weight of the anhydrate crystal form (Form B) of 2-(3-fluoro-4-hydroxyphenyl)-7-vinyl-1,3-benzoxazol-5-ol. In some embodiments of the solid formulations, the pharmaceutical formulations further comprises an additional active ingredient such as a progestin.

In some embodiments of the solid formulations, the active pharmacological agent comprises from about 0.01% to about 80% by weight of the pharmaceutical formulation. In some embodiments of the solid formulations, the active pharmacological agent comprises from about 0.01% to about 75% by weight of the pharmaceutical formulation. In some embodiments of the solid formulations, the active pharmacological agent comprises from about 0.01% to about 50% by weight of the pharmaceutical formulation. In some embodiments of the solid formulations, the active pharmacological agent comprises from about 0.1% to about 50% by weight of the pharmaceutical formulation. In some embodiments of the solid formulations, the active pharmacological agent comprises from about 0.1% to about 40% by weight of the pharmaceutical formulation. In some embodiments of the solid formulations, the active pharmacological agent comprises from about 0.1% to about 30% by weight of the pharmaceutical formulation. In some embodiments of the solid formulations, the active pharmacological agent comprises from about 0.1% to about 20% by weight of the pharmaceutical formulation. In some embodiments of the solid formulations, the active pharmacological agent comprises from about 1% to about 40% by weight of the pharmaceutical formulation. In some embodiments of the solid formulations, the active pharmacological agent comprises from about 1% to about 35% by weight of the pharmaceutical formulation. In some embodiments of the solid formulations, the active pharmacological agent comprises from about 1% to about 25% by weight of the pharmaceutical formulation. In some embodiments of the solid formulations, the active pharmacological agent comprises from about 1% to about 10% by weight of the pharmaceutical formulation. In some embodiments of the solid formulations, the active pharmacological agent comprises from about 10% to about 35% by weight of the pharmaceutical formulation. In some embodiments of the solid formulations, the active pharmacological agent comprises from about 1% to about 10% by weight of the pharmaceutical formulation. In some embodiments of the solid formulations, the active pharmacological agent comprises from about 10% to about 30% by weight of the pharmaceutical formulation. In some embodiments of the solid formulations, the active pharmacological agent comprises about 5% by weight of the pharmaceutical formulation. In some embodiments of the solid formulations, the active pharmacological agent comprises about 25% by weight of the pharmaceutical formulation.

In some embodiments of the solid formulations, the first diluent filler component comprises from about 30% to about 95% by weight of the pharmaceutical formulation. In some embodiments of the solid formulations, the first diluent filler component comprises from about 38% to about 95% by weight of the pharmaceutical formulation. In some embodiments of the solid formulations, the first diluent filler component comprises from about 40% to about 80% by weight of the pharmaceutical formulation. In some embodiments of the solid formulations, the first diluent filler component comprises from about 40% to about 60% by weight of the pharmaceutical formulation. In some embodiments of the solid formulations, the first diluent filler component comprises from about 60% to about 80% by weight of the pharmaceutical formulation. In some embodiments of the solid formulations, the first diluent filler component comprises from about 45% to about 55% by weight of the pharmaceutical formulation. In some embodiments of the solid formulations, the first diluent filler component comprises from about 65% to about 75% by weight of the pharmaceutical formulation. In some embodiments of the solid formulations, the first diluent filler component comprises from about 51.5% by weight of the pharmaceutical formulation. In some embodiments of the solid formulations, the first diluent filler component comprises from about 71.5% by weight of the pharmaceutical formulation.

In some embodiments of the solid formulations, the optional second diluent filler component, when present, comprises up to about 40% by weight of the pharmaceutical formulation. In some embodiments of the solid formulations, the optional second diluent filler component, when present, comprises up to about 30% by weight of the pharmaceutical formulation. In some embodiments of the solid formulations, the optional second diluent filler component, when present, comprises up to about 25% by weight of the pharmaceutical formulation. In some embodiments of the solid formulations, the optional second diluent filler component, when present, comprises up to about 20% by weight of the pharmaceutical formulation. In some embodiments of the solid formulations, the optional second diluent filler component, when present, comprises from about 5% to about 25% by weight of the pharmaceutical formulation. In some embodiments of the solid formulations, the optional second diluent filler component, when present, comprises from about 10% to about 20% by weight of the pharmaceutical formulation. In some embodiments of the solid formulations, the optional second diluent filler component, when present, comprises from about 5% to about 20% by weight of the pharmaceutical formulation. In some embodiments of the solid formulations, the optional second diluent filler component, when present, comprises about 15% by weight of the pharmaceutical formulation. In some embodiments of the solid formulations, the optional second diluent filler component, when present, comprises about 5% by weight of the pharmaceutical formulation. In some embodiments of the solid formulations, the optional second diluent/filler component, when present, comprises about 25% by weight of the pharmaceutical formulation.

In some embodiments of the solid formulations, the disintegrant component comprises from about 0.01% to about 30% by weight of the pharmaceutical formulation. In some embodiments of the solid formulations, the disintegrant component comprises from about 0.01% to about 20% by weight of the pharmaceutical formulation. In some embodiments of the solid formulations, the disintegrant component comprises from about 0.5% to about 20% by weight of the pharmaceutical formulation. In some embodiments of the solid formulations, the disintegrant component comprises from about 0.1% to about 20% by weight of the pharmaceutical formulation. In some embodiments of the solid formulations, the disintegrant component comprises from about 1% to about 20% by weight of the pharmaceutical formulation. In some embodiments of the solid formulations, the disintegrant component comprises from 1% to about 10% by weight of the pharmaceutical formulation. In some embodiments of the solid formulations, the disintegrant component comprises from about 0.5% to about 10% by weight of the pharmaceutical formulation. In some embodiments of the solid formulations, the disintegrant component comprises from about 1% to about 7% by weight of the pharmaceutical formulation. In some embodiments of the solid formulations, the disintegrant component comprises from about 3% to about 5% by weight of the pharmaceutical formulation. In some embodiments of the solid formulations, the disintegrant component comprises from about 2% to about 6% by weight of the pharmaceutical formulation. In some embodiments of the solid formulations, the disintegrant component comprises from about 1% to about 3% by weight of the pharmaceutical formulation. In some embodiments of the solid formulations, the disintegrant component comprises about 4% by weight of the pharmaceutical formulation. In some embodiments of the solid formulations, the disintegrant component comprises about 2% by weight of the pharmaceutical formulation. In some embodiments of the solid formulations, the disintegrant component comprises about 6% by weight of the pharmaceutical formulation.

In some embodiments of the solid formulations, the binder component comprises from about 0.01% to about 20% by weight of the pharmaceutical formulation. In some embodiments of the solid formulations, the binder component comprises from about 0.01% to about 10% by weight of the pharmaceutical formulation. In some embodiments of the solid formulations, the binder component comprises from about 0.1% to about 10% by weight of the pharmaceutical formulation. In some embodiments of the solid formulations, the binder component comprises from about 0.5% to about 10% by weight of the pharmaceutical formulation. In some embodiments of the solid formulations, the binder component comprises from about 1% to about 10% by weight of the pharmaceutical formulation. In some embodiments of the solid formulations, the binder component comprises from about 1% to about 8% by weight of the pharmaceutical formulation. In some embodiments of the solid formulations, the binder component comprises from about 1% to about 7% by weight of the pharmaceutical formulation. In some embodiments of the solid formulations, the binder component comprises from about 1% to about 6% by weight of the pharmaceutical formulation. In some embodiments of the solid formulations, the binder component comprises from about 0.5% to about 5% by weight of the pharmaceutical formulation. In some embodiments of the solid formulations, the binder component comprises from about 1% to about 5% by weight of the pharmaceutical formulation. In some embodiments of the solid formulations, the binder component comprises from about 1% to about 3% by weight of the pharmaceutical formulation. In some embodiments of the solid formulations, the binder component comprises about 2% by weight of the pharmaceutical formulation. In some embodiments of the solid formulations, the binder component comprises about 1% by weight of the pharmaceutical formulation. In some embodiments of the solid formulations, the binder component comprises about 3% by weight of the pharmaceutical formulation.

In some embodiments of the solid formulations, the wetting agent component comprises from about 0.01% to about 20% by weight of the pharmaceutical formulation. In some embodiments of the solid formulations, the wetting agent component comprises from about 0.01% to about 10% by weight of the pharmaceutical formulation. In some embodiments of the solid formulations, the wetting agent component comprises from about 0.1% to about 20% by weight of the pharmaceutical formulation. In some embodiments of the solid formulations, the wetting agent component comprises from about 0.1% to about 10% by weight of the pharmaceutical formulation. In some embodiments of the solid formulations, the wetting agent component comprises from about 1% to about 20% by weight of the pharmaceutical formulation. In some embodiments of the solid formulations, the wetting agent component comprises from about 0.01% to about 10% by weight of the pharmaceutical formulation. In some embodiments of the solid formulations, the wetting agent component comprises from about 1% to about 8% by weight of the pharmaceutical formulation. In some embodiments of the solid formulations, the wetting agent component comprises from about 0.5% to about 8% by weight of the pharmaceutical formulation. In some embodiments of the solid formulations, the wetting agent component comprises from about 0.01% to about 20% by weight of the pharmaceutical formulation. In some embodiments of the solid formulations, the wetting agent component comprises from about 1.3% to about 5% by weight of the pharmaceutical formulation. In some embodiments of the solid formulations, the wetting agent component comprises from about 1.3% to about 4% by weight of the pharmaceutical formulation. In some embodiments of the solid formulations, the wetting agent component comprises from about 1.5% to about 5% by weight of the pharmaceutical formulation. In some embodiments of the solid formulations, the wetting agent component comprises from about 1.5% to about 4% by weight of the pharmaceutical formulation. In some embodiments of the solid formulations, the wetting agent component comprises from about 1% to about 3% by weight of the pharmaceutical formulation. In some embodiments of the solid formulations, the wetting agent component comprises about 2% by weight of the pharmaceutical formulation. In some embodiments of the solid formulations, the wetting agent component comprises about 1% by weight of the pharmaceutical formulation. In some embodiments of the solid formulations, the wetting agent component comprises about 3% by weight of the pharmaceutical formulation. In some embodiments of the solid formulations, the wetting agent component comprises about 4% by weight of the pharmaceutical formulation. In some embodiments of the solid formulations, the wetting agent component comprises from about 5% by weight of the pharmaceutical formulation.

In some embodiments of the solid formulations, the optional lubricant component, when present, comprises from about 0.01% to about 10% by weight of the pharmaceutical formulation. In some embodiments of the solid formulations, the optional lubricant component, when present, comprises from about 0.01% to about 5% by weight of the pharmaceutical formulation. In some embodiments of the solid formulations, the optional lubricant component, when present, comprises from about 0.01% to about 2% by weight of the pharmaceutical formulation. In some embodiments of the solid formulations, the optional lubricant component, when present, comprises from about 0.01% to about 1% by weight of the pharmaceutical formulation. In some embodiments of the solid formulations, the optional lubricant component, when present, comprises from about 0.1% to about 10% by weight of the pharmaceutical formulation. In some embodiments of the solid formulations, the optional lubricant component, when present, comprises from about 0.1% to about 5% by weight of the pharmaceutical formulation. In some embodiments of the solid formulations, the optional lubricant component, when present, comprises from about 0.1% to about 2% by weight of the pharmaceutical formulation. In some embodiments of the solid formulations, the optional lubricant component, when present, comprises from about 0.1% to about 1% by weight of the pharmaceutical formulation. In some embodiments of the solid formulations, the optional lubricant component, when present, comprises about 0.5% by weight of the pharmaceutical formulation.

In some embodiments of the solid formulations, the pharmaceutical formulation comprises from about 1 mg to about 200 mg of the active pharmacological agent. In some embodiments of the solid formulations, the pharmaceutical formulation comprises from about 1 mg to about 10 mg of the active pharmacological agent. In some embodiments of the solid formulations, the pharmaceutical formulation comprises from about 10 mg to about 50 mg of the active pharmacological agent. In some embodiments of the solid formulations, the pharmaceutical formulation comprises from about 50 mg to about 100 mg of the active pharmacological agent. In some embodiments of the solid formulations, the pharmaceutical formulation comprises from about 100 mg to about 200 mg of the active pharmacological agent.

In some embodiments of the solid formulations, the ratio of the disintegrant component to the binder component is about 5:1 to about 1:1. In some embodiments of the solid formulations, the ratio of the disintegrant component to the binder component is 5:1 to about 1.5:1, about 5:1 to about 2:1, about 5:1 to about 2.5:1, or about 5:1 to about 3:1. In some embodiments of the solid formulations, the ratio of the disintegrant component to the binder component is 4:1 to about 1.5:1, about 4:1 to about 2:1, about 4:1 to about 2.5:1, or about 4:1 to about 3:1. In some embodiments of the solid formulations, the ratio of the disintegrant component to the binder component is about 3:1 to about 1:1. In some embodiments of the solid formulations, the ratio of the disintegrant component to the binder component is about 2:1 to about 1:1. In some embodiments of the solid formulations, the ratio of the disintegrant component to the binder component is about 3:1 to about 1.5:1, about 3:1 to about 2:1, about 2.5:1 to about 1:1, or about 2.5:1 to about 1.5:1. In some embodiments of the solid formulations, the ratio of the disintegrant component to the binder component is about 6:1 to about 1:6, about 6:1 to about 5:1, about 6:1 to about 4:1, about 6:1 to about 3:1, about 6:1 to about 2:1, or about 6:1 to about 1:1. In some embodiments of the solid formulations, the ratio of the disintegrant component to the binder component is about 5:1, about 4:1, about 3:1, or about 2:1.

In some embodiments of the solid formulations, the ratio of the binder component to the wetting agent component is about 3:1 to about 1:3. In some embodiments of the solid formulations, the ratio of the binder component to the wetting agent component is about 3:1 to about 1:1. In some embodiments of the solid formulations, the ratio of the binder component to the wetting agent component is about 2:1 to about 1:1. In some embodiments of the solid formulations, the ratio of the binder component to the wetting agent component is about 3:1 to about 1:2, about 3:1 to about 1.5:1, or about 2.5:1 to about 1.5:1. In some embodiments of the solid formulations, the ratio of the disintegrant component to the binder component is about 1:1 to about 1:3, about 1:1.5 to about 1:3, about 1:2 to about 1:3, or about 1:2.5 to about 1:3. In some embodiments of the solid formulations, the ratio of the binder component to the wetting agent component is about to about 1:1, about 2:1, about 1:2, about 3:1, or about 1:3.

In some embodiments of the solid formulations, the ratio of the disintegrant component to the binder component to the wetting agent component is about 6:1:1 to about 1:1:1. In some embodiments of the solid formulations, the ratio of the disintegrant component to the binder component to the wetting agent component is about 5:1:1. In some embodiments of the solid formulations, the ratio of the disintegrant component to the binder component to the wetting agent component is about 4:1:1. In some embodiments of the solid formulations, the ratio of the disintegrant component to the binder component to the wetting agent component is about 3:1:1. In some embodiments of the solid formulations, the ratio of the disintegrant component to the binder component to the wetting agent component is about 2:1:1.

In some embodiments of the solid formulations, when the pharmaceutical formulation comprises one or more ingredients selected from metallic lauryl sulfate, sodium lauryl sulfate, metal alkyl sulfate, polyethylene glycol, glyceride of fatty ester, Poloxamer 188, polyoxyethylene sorbitan fatty acid ester, polyoxyethylene castor oil derivative, sugar ester of fatty acid, polyglycolized glyceride, quaternary ammonium amine compound, and docusate sodium, then the sum of the amounts of the ingredients does not exceed about 15% by weight of the pharmaceutical formulation.

In some embodiments of the solid formulations, when the pharmaceutical formulation comprises one or more ingredients selected from metallic lauryl sulfate, sodium lauryl sulfate, metal alkyl sulfate, polyethylene glycol, glyceride of fatty ester, Poloxamer 188, polyoxyethylene sorbitan fatty acid ester, polyoxyethylene castor oil derivative, sugar ester of fatty acid, polyglycolized glyceride, quaternary ammonium amine compound, and docusate sodium, then the sum of the amounts of the ingredients does not exceed about 10% by weight of the pharmaceutical formulation.

In some embodiments of the solid formulations, when the pharmaceutical formulation comprises one or more ingredients selected from metallic lauryl sulfate, sodium lauryl sulfate, metal alkyl sulfate, polyethylene glycol, glyceride of fatty ester, Poloxamer 188, polyoxyethylene sorbitan fatty acid ester, polyoxyethylene castor oil derivative, sugar ester of fatty acid, polyglycolized glyceride, quaternary ammonium amine compound, and docusate sodium, then the sum of the amounts of the ingredients does not exceed about 8% by weight of the pharmaceutical formulation.

In some embodiments of the solid formulations, when the pharmaceutical formulation comprises one or more ingredients selected from metallic lauryl sulfate, sodium lauryl sulfate, metal alkyl sulfate, polyethylene glycol, glyceride of fatty ester, Poloxamer 188, polyoxyethylene sorbitan fatty acid ester, polyoxyethylene castor oil derivative, sugar ester of fatty acid, polyglycolized glyceride, quaternary ammonium amine compound, and docusate sodium, then the sum of the amounts of the ingredients does not exceed about 5% by weight of the pharmaceutical formulation.

In some embodiments of the solid formulations, when the pharmaceutical formulation comprises one or more ingredients selected from metallic lauryl sulfate, sodium lauryl sulfate, metal alkyl sulfate, polyethylene glycol, glyceride of fatty ester, Poloxamer 188, polyoxyethylene sorbitan fatty acid ester, polyoxyethylene castor oil derivative, sugar ester of fatty acid, polyglycolized glyceride, quaternary ammonium amine compound, and docusate sodium, then the sum of the amounts of the ingredients does not exceed about 4% by weight of the pharmaceutical formulation.

In some embodiments of the solid formulations, each optional component is present in the formulation.

In some embodiments of the solid formulations, each optional component comprises only one material.

In some embodiments of the solid formulations, the first diluent/filler component, the optional second diluent/filler component, if present, the disintegrant component, the binder component, the wetting agent component, and the optional lubricant component, if present, are different materials.

As used herein, the term “first diluent/filler component” refers to one or more substances that act to dilute the active pharmacological agent to the desired dosage and/or that act as a carrier for the active pharmacological agent. In some embodiments of the solid formulations, the first diluent/filler component comprises one or more filler substances. In some embodiments of the solid formulations, the first diluent/filler component comprises one or more diluent substances. In some embodiments of the solid formulations, the first diluent/filler component comprises one or more substances that are diluents and fillers. In some embodiments, the first diluent/filler component comprises at least one a substance that improves the mechanical strength and/or compressibility of the pharmaceutical compositions of the invention.

In some embodiments of the solid formulations, the first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments of the solid formulations, the first diluent/filler component is mannitol.

As used herein, the term “second diluent/filler component” refers to one or more substances that act to dilute the active pharmacological agent to the desired dosage and/or that act as a carrier for the active pharmacological agent. In some embodiments of the solid formulations, the second diluent/filler component comprises one or more filler substances. In some embodiments of the solid formulations, the second diluent/filler component comprises one or more diluent substances. In some embodiments of the solid formulations, the second diluent/filler component comprises one or more substances that are diluents and fillers. In some embodiments, the second diluent/filler component comprises at least one substance that improves the mechanical strength and/or compressibility of the pharmaceutical compositions of the invention.

In some embodiments of the solid formulations, the optional second diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments of the solid formulations, the optional second diluent/filler component, when present, comprises microcrystalline cellulose.

As used herein, the term “disintegrant component” refers to one or more substances that encourage disintegration in water (or water-containing fluid in vivo) of a pharmaceutical composition comprising the pharmaceutical formulations of the invention.

In some embodiments of the solid formulations, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments of the solid formulations, the disintegrant component comprises croscarmellose sodium.

As used herein, the term “binder component” refers to one or more substances that increase the mechanical strength and/or compressibility of a pharmaceutical composition comprising the pharmaceutical formulations of the invention.

In some embodiments of the solid formulations, the binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol.

In some embodiments of the solid formulations, the binder component comprises one or more of binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, or kaolin.

In some embodiments of the solid formulations, the binder component comprises polyvinylpyrrolidone. In some embodiments of the solid formulations, the binder component comprises povidone K12, K17, K25, K30, K60, K90, or K120. In some embodiments of the solid formulations, the binder component comprises povidone K25.

As used herein, the term “wetting agent component” refers to one or more substances that increase the water permeability of pharmaceutical compositions comprising the pharmaceutical formulations of the invention. In another aspect, the term, “wetting agent component” refers to one or more substances that increase dissolution of the active pharmacological agent in water (or water containing fluid in vivo). In yet another aspect, the term “wetting agent component” refers to one or more substances that increase the bioavailability of the active pharmacological agent after administration of the pharmaceutical compositions and formulations of the invention.

In some embodiments of the solid formulations, the wetting agent component comprises one or more of one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium.

In some embodiments of the solid formulations, the wetting agent component comprises one or more of polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated glycerol fatty acid ester, polyethoxylated fatty acid ester, or docusate sodium.

In some embodiments of the solid formulations, the wetting agent component comprises metal alkyl sulfate. In some embodiments of the solid formulations, the wetting agent component comprises metallic lauryl sulfate. In some embodiments of the solid formulations, the wetting agent component comprises sodium lauryl sulfate.

As used herein, the term “lubricant component” refers to one or more substances that aids in preventing sticking to the equipment of the pharmaceutical formulations during processing and/or that improves powder flow of the formulation during processing.

In some embodiments of the solid formulations, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

In some embodiments of the solid formulations, optional lubricant component, when present, comprises metallic stearate. In some embodiments of the solid formulations, optional lubricant component, when present, comprises one or more of zinc stearate, calcium stearate, magnesium stearate, or sodium stearate. In some embodiments of the solid formulations, the optional lubricant component, when present, comprises magnesium stearate.

In some embodiments of the solid formulations:

(a) the first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate;

(b) the second optional diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, pregelatinized starch, sodium starch glycolate, a calcium phosphate, a metal carbonate, a metal oxide, or a metal aluminosilicate;

(c) the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate;

(d) the binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol;

(e) the wetting agent component comprises one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium; and

(f) the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

In some embodiments of the solid formulations:

(a) the first diluent/filler component comprises mannitol;

(b) the second optional diluent/filler component, when present, comprises microcrystalline cellulose;

(c) the disintegrant component comprises croscarmellose sodium;

(d) the binder component comprises polyvinylpyrrolidone;

(e) the wetting agent component comprises sodium lauryl sulfate; and

(f) the optional lubricant component, when present, comprises magnesium stearate.

The present invention is also directed to processes for producing the solid pharmaceutical formulations of the invention. In one aspect, the process utilize direct blend techniques for producing the solid pharmaceutical formulations of the invention. In another aspect, the processes utilize wet granulation techniques for producing the solid pharmaceutical formulations of the invention. In further aspect, the present invention is directed to dry granulation processes for producing the solid pharmaceutical formulations of the invention. Granulation of the solid pharmaceutical formulations can be accomplished by any of the granulation techniques known to one of skill in the art. For example, dry granulation techniques include, but are not limited to, compression of the mixed powder under high pressure, either by roller compaction or “slugging” in a heavy-duty tablet press. Wet granulation techniques include, but are not limited to, high shear granulation, single-pot processing, top-spray granulation, bottom-spray granulation, fluidized spray granulation, extrusion/spheronization, and rotor granulation.

Accordingly, the present invention provides a process for preparing the solid pharmaceutical formulations of the invention comprising:

(a) mixing the active pharmacological agent with the first diluent/filler component, the disintegrant component, and the optional second filler/diluent component, if present, to form an initial mixture; and

(b) granulating the initial mixture with an aqueous solution comprising the wetting agent component to form a granulated mixture.

In some embodiments, the step (a) comprises:

(i) mixing the active pharmacological agent with at least a portion of the first diluent/filler component to form a first mixture; and

(ii) mixing the first mixture with the remainder of the first diluent/filler component, if any, the disintegrant component, and the optional second filler/diluent component, if present, to form the initial mixture.

In some embodiments, the aqueous solution further comprises the binder component.

In some embodiments, the process further comprises:

(i) drying the granulated mixture to form a dried granulated mixture; and

(ii) mixing the optional lubricant component, if present, with the dried granulated mixture to form a final mixture.

In some embodiments, the step (ii) comprises:

(a) mixing the optional lubricant component, if present, with a portion of the dried granulated mixture; and

(b) mixing the mixture from (i) with the remainder of the dried granulated mixture.

In some embodiments, step (ii)(b) is carried out in a blender.

In some embodiments, the process comprises:

(i) mixing the active pharmacological agent with at least a portion of the first diluent/filler component to form a first mixture;

(ii) mixing the first mixture with the remainder of the first diluent/filler component, if any, the disintegrant component, and the optional second filler/diluent component, if present, to form the initial mixture;

(iii) granulating the initial mixture with an aqueous solution comprising the wetting agent component to form a granulated mixture

(iv) drying the granulated mixture to form a dried granulated mixture;

(v) mixing the optional lubricant component, if present, with the at least a portion of the dried granulated mixture; and

(vi) mixing the mixture from (v) with the remainder of the dried granulated mixture, if any.

In some embodiments, the aqueous solution further comprises the binder component.

In some embodiments where wet granulation technique is employed (i.e., an aqueous solution comprising the wetting agent component is used), the solid pharmaceutical formulations obtained may contain up to 5% by weight of water (even after the step of drying the granulated mixture to form a dried granulated mixture). In some embodiments, the solid pharmaceutical formulations of the present invention may contain up to 5%, up to 4%, up to 3%, up to 2%, up to 1%, up to 0.5%, up to 0.2% or up to 0.1% by weight of water (moisture). In some embodiments, the solid pharmaceutical formulations of the present invention contain less than 5%, 4%, 3%, 2%, 1%, 0.5%, 0.2% or 0.1% by weight of water (moisture).

The processes described herein can be used to prepare any of the solid pharmaceutical formulations described herein, as well as any combination and subcombinations of the embodiments thereof.

In some embodiments:

(a) the first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate;

(b) the second optional diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, pregelatinized starch, sodium starch glycolate, a calcium phosphate, a metal carbonate, a metal oxide, or a metal aluminosilicate;

(c) the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate;

(d) the binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol;

(e) the wetting agent component comprises one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium; and

(f) the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

In some embodiments:

(a) the first diluent/filler component comprises mannitol;

(b) the second optional diluent/filler component, when present, comprises microcrystalline cellulose;

(c) the disintegrant component comprises croscarmellose sodium;

(d) the binder component comprises polyvinylpyrrolidone;

(e) the wetting agent component comprises sodium lauryl sulfate; and

(f) the optional lubricant component, when present, comprises magnesium stearate.

The invention further provides a process for producing the solid pharmaceutical formulations of the invention comprising:

(i) mixing the first diluent/filler component, the optional second diluent/filler component, if present, the disintegrant component, the binder component, the wetting agent component, and the active pharmacological agent to form a first mixture; and

ii) optionally granulating the first mixture.

The process described herein can be used to prepare any of the solid pharmaceutical formulations described herein, as well as any combination and subcombinations of the embodiments thereof. In some embodiments, the first mixture further comprises the optional lubricant component.

The present invention further provides products of the processes for preparing the solid pharmaceutical formulation of the invention.

The present invention further provides tablets comprising the solid pharmaceutical formulations of the invention. Any of the pharmaceutical formulations described herein, as well as any combination and subcombinations of the embodiments thereof, can be used to prepare the tablets of the invention.

The present invention further provides a process for producing the tablets of the invention comprising compressing the solid pharmaceutical formulations of the invention. In some embodiments, the process further comprises milling the solid pharmaceutical formulation prior to the compressing of the solid pharmaceutical formulation.

In some embodiments, the compressing yields a tablet of about 7 Kp to about 13 Kp hardness. In some embodiments, the tablet has a hardness of about 7 Kp to about 13 Kp.

In some embodiments, the anhydrate crystal form (Form B) of 2-(3-fluoro-4-hydroxyphenyl)-7-vinyl-1,3-benzoxazol-5-ol which is present in the solid formulation remains substantially intact (i.e., no conversion to another crystal form or an amorphous form) during processing, transportation and/or storage under ambient conditions for a period greater than about 36 hours, greater than about 1 week, greater than about 1 month, greater than about 6 months, or greater than about 1 year.

Certain features of the invention are described herein in embodiments. It is emphasized that certain features of the invention, which are, for clarity, described herein in the context of separate embodiments, can also be provided in combination in a single embodiment. Conversely, various features of the invention which are, for brevity, described in the context of a single embodiment, can also be provided separately or in any suitable subcombination. For example, some of the embodiments herein describe individual weight percentages for each component in the pharmaceutical formulations, while other embodiments herein describe the chemical composition of the components of the pharmaceutical formulations; these embodiments can also be provided in any suitable combination or subcombination, as well as being provided separately in a single embodiment. These statements apply both to the liquid or semi-solid pharmaceutical formulations, as well as to the solid pharmaceutical formulations, and compositions, products, and processes thereof.

It will be understood that the weight percentages set forth for the components of the pharmaceutical formulations disclosed herein are the percentages that each component will comprise of a final pharmaceutical formulation, without reference to any surface covering, such as a tablet coating or capsule. The remainder of the final formulation will be comprised of the active pharmacological agent(s).

### DEFINITIONS

As used herein, the term “alginic acid” refers to a naturally occurring hydrophilic colloidal polysaccharide obtained from the various species of seaweed, or synthetically modified polysaccharides thereof.

As used herein, the term “sodium alginate” refers to a sodium salt of alginic acid and can be formed by reaction of alginic acid with a sodium containing base such as sodium hydroxide or sodium carbonate. As used herein, the term “potassium alginate” refers to a potassium salt of alginic acid and can be formed by reaction of alginic acid with a potassium containing base such as potassium hydroxide or potassium carbonate. As used herein, the term “calcium alginate” refers to a calcium salt of alginic acid and can be formed by reaction of alginic acid with a calcium containing base such as calcium hydroxide or calcium carbonate. Suitable sodium alginates, calcium alginates, and potassium alginates include, but are not limited to, those described in R. C. Rowe and P. J. Shesky, Handbook of pharmaceutical excipients, (2006), 5th ed., which is hereby incorporated by reference in its entirety. Suitable sodium alginates, include, but are not limited to, Kelcosol (available from ISP), Kelfone LVCR and HVCR (available from ISP), Manucol (available from ISP), and Protanol (available from FMC Biopolymer).

As used herein, the term “amino acid” refers to any known amino acid. Suitable amino acids include, but are not limited to, leucine.

As used herein, the term “calcium silicate” refers to a silicate salt of calcium.

As used herein, the term “calcium phosphate” refers to monobasic calcium phosphate, dibasic calcium phosphate or tribasic calcium phosphate.

As used herein, the term “caprylocaproyl macrogolglyceride” refers to a polyglycolized glyceride synthesized predominately from a mixture of capric acid and caprylic acid or from compounds derived predominately from a mixture of capric acid and caprylic acid, although other fatty acids or compounds derived from other fatty acids may used in the synthesis as well. Suitable caprylocaproyl macrogolglycerides include, but are not limited to, Labrasol™ (available from Gattefossé).

Cellulose, cellulose floc, powdered cellulose, microcrystalline cellulose, silicified microcrystalline cellulose, carboxyethylcellulose, carboxymethylcellulose, hydroxyethylcellulose, methylhydroxyethylcellulose, hydroxymethylcellulose, hydroxypropylcellulose, hydroxypropylmethylcellulose, hydroxypropylmethylcellulose phthalate, ethylcellulose, methylcellulose, carboxymethylcellulose sodium, and carboxymethyl cellulose calcium include, but are not limited to, those described in R. C. Rowe and P. J. Shesky, Handbook of pharmaceutical excipients, (2006), 5th ed., which is hereby incorporated by reference in its entirety. As used herein, cellulose refers to natural cellulose. The term “cellulose” also refers to celluloses that have been modified with regard to molecular weight and/or branching, particularly to lower molecular weight. The term “cellulose” further refers to celluloses that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable powdered celluloses for use in the invention include, but are not limited to Arbocel (available from JRS Pharma), Sanacel (available from CFF GmbH), and Solka-Floc (available from International Fiber Corp.).

Suitable microcrystalline celluloses include, but are not limited to, the Avicel pH series (available from FMC Biopolymer), Celex (available from ISP), Celphere (available from Asahi Kasei), Ceolus KG (available from Asahi Kasei), and Vivapur (available from JRS Pharma).

As used herein, the term “silicified microcrystalline cellulose” refers to a synergistic intimate physical mixture of silicon dioxide and microcrystalline cellulose. Suitable silicified microcrystalline celluloses include, but are not limited to, ProSolv (available from JRS Pharma).

As used herein, the term “carboxymethylcellulose sodium” refers to a cellulose ether with pendant groups of formula Na⁺ ⁻O—C(O)—CH₂—, attached to the cellulose via an ether linkage. Suitable carboxymethylcellulose sodium polymers include, but are not limited to, Akucell (available from Akzo Nobel), Aquasorb (available from Hercules), Blanose (available from Hercules), Finnfix (available from Noviant), Nymel (available from Noviant), and Tylose CB (available from Clariant).

As used herein, the term “carboxymethylcellulose calcium” refers to a cellulose ether with a pendant groups of formula —CH₂—O—C(O)—O⁻ ½ Ca²⁺, attached to the cellulose via an ether linkage.

As used herein, the term “carboxymethylcellulose” refers to a cellulose ether with pendant carboxymethyl groups of formula HO—C(O)—CH₂—, attached to the cellulose via an ether linkage. Suitable carboxymethylcellulose calcium polymers include, but are not limited to, Nymel ZSC (available from Noviant).

As used herein, the term “carboxyethylcellulose” refers to a cellulose ether with pendant carboxymethyl groups of formula HO—C(O)—CH₂—CH₂—, attached to the cellulose via an ether linkage.

As used herein, the term “hydroxyethylcellulose” refers to a cellulose ether with pendant hydroxyethyl groups of formula HO—CH₂—CH₂—, attached to the cellulose via an ether linkage. Suitable hydroxyethylcelluloses include, but are not limited to, Cellosize HEC (available from DOW), Natrosol (available from Hercules), and Tylose PHA (available from Clariant).

As used herein, the term “methylhydroxyethylcellulose” refers to a cellulose ether with pendant methyloxyethyl groups of formula CH₃—O—CH₂—CH₂—, attached to the cellulose via an ether linkage. Suitable methylhydroxyethylcelluloses include, but are not limited to, the Culminal MHEC series (available from Hercules), and the Tylose series (available from Shin Etsu).

As used herein, the term “hydroxypropylcellulose”, or “hypomellose”, refers a cellulose that has pendant hydroxypropoxy groups, and includes both high- and low-substituted hydroxypropylcellulose. In some embodiments, the hydroxypropylcellulose has about 5% to about 25% hydroxypropyl groups. Suitable hydroxypropylcelluloses include, but are not limited to, the Klucel series (available from Hercules), the Methocel series (available from Dow), the Nisso HPC series (available from Nisso), the Metolose series (available from Shin Etsu), and the LH series, including LHR-11, LH-21, LH-31, LH-20, LH-30, LH-22, and LH-32 (available from Shin Etsu).

As used herein, the term “methyl cellulose” refers to a cellulose that has pendant methoxy groups. Suitable methyl celluloses include, but are not limited to Culminal MC (available from Hercules).

As used herein, the term “ethyl cellulose” refers to a cellulose that has pendant ethoxy groups. Suitable ethyl celluloses include, but are not limited to Aqualon (available from Hercules).

As used herein, the term “carmellose calcium” refers to a crosslinked polymer of carboxymethylcellulose calcium.

As used herein, the term “copovidone” refers to a copolymer of vinylpyrrolidone and vinyl acetate, wherein the vinyl acetate monomers may be partially hydrolyzed. Suitable copovidone polymers include, but are not limited to Kollidon VA 64 (available from BASF, Luviskol VA (available from BASF, Plasdone S-630 (available from ISP), and Majsao CT (available from Cognis).

As used herein, the term “croscarmellose sodium” refers to a crosslinked polymer of carboxymethylcellulose sodium.

As used herein, the term “crospovidone” refers to a crosslinked polymer of polyvinylpyrrolidone. Suitable crospovidone polymers include, but are not limited to Polyplasdone XL-10 (available from ISP) and Kollidon CL and CL-M (available from BASF).

As used herein, the term “crosslinked poly(acrylic acid)” refers to a polymer of acrylic acid which has been crosslinked. The crosslinked polymer may contain other monomers in addition to acrylic acid. Additionally, the pendant carboxy groups on the crosslinked polymer may be partially or completely neutralized to form a pharmaceutically acceptable salt of the polymer. In some embodiments, the crosslinked poly(acrylic acid) is neutralized by ammonia or sodium hydroxide. Suitable crosslinked poly(acrylic acid) polymers include, but are not limited to, the Carbopol series (available from Noveon).

As used herein, the term “an effervescent system based on food acids and an alkaline carbonate component” refers to a excipient combination of food acids and alkaline carbonates that releases carbon dioxide gas when administered. Suitable effervescent systems are those that those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.).

As used herein, the term “fatty acid” refers to an aliphatic acid that is saturated or unsaturated. In some embodiments, the fatty acid in a mixture of different fatty acids. In some embodiments, the fatty acid has between about eight to about thirty carbons on average. In some embodiments, the fatty acid has about eight to about twenty-four carbons on average. In some embodiments, the fatty acid has about twelve to about eighteen carbons on average. Suitable fatty acids include, but are not limited to, stearic acid, lauric acid, myristic acid, erucic acid, palmitic acid, palmitoleic acid, capric acid, caprylic acid, oleic acid, linoleic acid, linolenic acid, hydroxystearic acid, 12-hydroxystearic acid, cetostearic acid, isostearic acid, sesquioleic acid, sesqui-9-octadecanoic acid, sesquiisooctadecanoic acid, benhenic acid, isobehenic acid, and arachidonic acid, or mixtures thereof. Other suitable fatty alcohols include, but are not limited, the Hystrene® series (available from Humko).

As used herein, the term “salt of a fatty acid” refers to a pharmaceutically acceptable salt derived from the reaction of a fatty acid with a base. As used herein, the phrase “pharmaceutically acceptable” refers to a substance that is acceptable for use in pharmaceutical applications from a toxicological perspective and does not adversely interact with the active ingredient. In some embodiments, the salt is sodium, potassium, calcium, or ammonium. Useful fatty acids for deriving the salts include, but are not limited to, those described herein. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in their entireties.

As used herein, the term “fatty alcohol” refers to an aliphatic alcohol that is saturated or unsaturated. In some embodiments, the fatty alcohol in a mixture of different fatty alcohols. In some embodiments, the fatty alcohol has between about eight to about thirty carbons on average. In some embodiments, the fatty alcohol has about eight to about twenty-four carbons on average. In some embodiments, the fatty alcohol has about twelve to about eighteen carbons on average. Suitable fatty alcohols include, but are not limited to, stearyl alcohol, lauryl alcohol, palmityl alcohol, palmitolyl acid, cetyl alcohol, capryl alcohol, caprylyl alcohol, oleyl alcohol, linolenyl alcohol, arachidonic alcohol, behenyl alcohol, isobehenyl alcohol, selachyl alcohol, chimyl alcohol, and linoleyl alcohol, or mixtures thereof.

As used herein, the term “fatty ester” refers to an ester compound formed between a fatty acid and an organic compound containing a hydroxyl group. In some embodiments, hydroxyl group containing compound is a carbohydrate, such as, but not limited to, glucose, lactose, sucrose, dextrose, mannitol, xylitol, sorbitol, maltodextrin and the like. In some embodiments, the hydroxyl containing compound is a fatty alcohol. In some embodiments, the fatty ester comprises lanolin. In some embodiments, the fatty ester comprises capric ester or caprylic esters, or mixtures thereof. In some embodiments, the fatty ester comprises about 95% or greater of saturated fatty esters. Suitable fatty acids and fatty alcohols for deriving the fatty esters include, but are not limited to, those defined herein. Suitable fatty esters include, but are not limited to sucrose fatty acid esters (such as those available from Mitsubishi Chemicals); ethyl oleate, Kessco™ EO (available from Akzo Nobel Chemical); medium chain triglycerides, Labrafac™ Lipo WL 1349 and CC (available from Gatefosse), capric triglycerides, caprylic triglycerides, and capric/caprylic triglycerides. Other suitable fatty esters include those listed in R. C. Rowe and P. J. Shesky, Handbook of pharmaceutical excipients, (2006), 5th ed., which is hereby incorporated by reference in its entirety. Medium chain fatty esters include, but are not limited, Labrafac™ CC (available from Gattefosse), Miglyol™ 810 and 812 (available from Multi Chem), the Myritol™ series (available from Cognis), Captex™ 300 and 355 (available from Abitec), and Crodamol™ GTC/C (available from Croda).

As used herein, the term “gelatin” refers to any material derived from boiling the bones, tendons, and/or skins of animals, or the material known as agar, derived from seaweed. The term “gelatin” also refers to any synthetic modifications of natural gelatin. Suitable gelatins include, but are not limited to, Byco (available from Croda Chemicals) and Cryogel and Instagel (available from Tessenderlo), and the materials described in R. C. Rowe and P. J. Shesky, Handbook of pharmaceutical excipients, (2006), 5th ed., which is hereby incorporated by reference in its entirety.

As used herein, the term “glycerides of fatty acid” refers to mono-, di- or triglycerides of fatty acids. The glycerides of fatty acid may be optionally substituted with sulfonic acid groups, or pharmaceutically acceptable salts thereof. Suitable fatty acids for deriving glycerides of fatty acids include, but are not limited to, those described herein. Glycerides of fatty acids useful in the present invention include, but are not limited to, Glyceryl monomyristate: Nikkol™ MGM (available from Nikko); Glyceryl monooleate: Peceol™ (available from Gattefosse), Hodag™ GMO-D, Nikkol™ MGO (Nikko); Glycerol monooleate/linoleate, Olicine™ (available from Gattefosse); Glycerol monolinoleate, Maisine™ 35-1 (Gattefosse), MYVEROL™ 18-92, Myverol™ 18-06 (available from Eastman); Glyceryl ricinoleate, Softigen™ 701 (available from Goldschmidt), Hodag™ GMR-D (available from Calgene), Aldo™ MR (available from Lonza); Glyceryl monolaurate: ALDO MLD (available from Lonza), Hodag™ GML (available from Calgene); Glycerol monopalmitate: Emalex™ GMS-P (available from Nihon); Glyceryl behenate, Compritol™ 888 ATO (Gattesfosse); Glyceryl monooleate: Aldo MO (available from Lonza), Atlas™ G-695 (available from Uniqema), Monomuls™ 90-018 (available from Cognis), Perceol™ (available from Gattefosse), Stepan™ GMO (available from Stepan Products), Rylo™ series (available from Danisco), Dimodan™ series (available from Danisco), Emuldan™ (available from Danisco) ADM™ DMG-40, 70, and 100 (available from ADM); Glycerol monostearate: Imwitor™ 900 (available from Sasol), Lipo™ GMS 410, 450, and 600 (available from Lipo Chemicals), Rita™ GMS (available from Rita Corp.), Stepan™ GMS (available from Stepan Products), Tegin™ (available from Goldschmidt), Kessco™ GMS (available from Akzo Nobel), Capmul™ GMS (available from Abitec), Myvaplex™ (available from Eastman), Cutina™ GMS, Aldo MS (available from Lonza), Nikkol™ MGS series (available from Nikko); Glyceryl plamitostearate: Precirol™ ATO J (available from Gattefosse); Glyceryl monodioleate: Capmul™ GMO-K (available from Abitec); Glyceryl palnitic/stearic: Cutina™ MD-A, ESTAGEL-G18; Glyceryl acetate: Lanegin™ EE (available from Grunau GmbH); Glyceryl laurate, Monomuls™ 90-45 (available from Cognis), Aldo™ MLD (available from Lonza); Glyceryl citrate/lactate/oleate/linoleate; Glyceryl caprylate: Capmul™ MCMC8 (available from Abitec); Glyceryl caprylate/caprate: Capmul™ MCM (available from Abitec); Caprylic acid mono, diglycerides; Caprylic/capric glycerides; Mono- and diacetylated monoglycerides, Myvacet™ 9-45, 9-40, and 9-08 (available from Eastman), Lamegin™ (available from Brenntag); Glyceryl monostearate, Aldo™ MS (available from Lonza), Lipo™ GMS (Lipo Chem.); Myvaplex™ (available from Eastman), Lactic acid esters of mono, diglycerides, Lamegin™ GLP (available from Brenntag); Glyceryl dilaurate: Capmul GDL (available from Abitec); Glyceryl dioleate: Capmul™ GDO (available from Abitec); and Glycerol esters of fatty acids: Gelucire® 39/01, 33/01, and 43/01 (available from Gattefosse). Other suitable glycerides of fatty acids include, but are not limited to, glyceryl monostearate, glyceryl monoisostearate, glyceryl monomyristate, glyceryl monooleate, diglyceryl monostearate, glyceryl behenate, and diglyceryl monoisostearate.

As used herein, the term “gum arabic” refers to natural, or synthetically modified, arabic gum. As used herein, the term “gum tragacanath” refers to natural, or synthetically modified, tragacanath gum. As used herein, the term “gum acacia” refers to natural, or synthetically modified, acacia gum. As used herein, the term “casein” refers to natural, or synthetically modified casein. As used herein, the term “kaolin” refers to natural, or synthetically modified, kaolin clay. Suitable gum arabic, gum tragacanath, gum acacia, casein, and kaolin include, but are not limited to, those described in R. C. Rowe and P. J. Shesky, Handbook of pharmaceutical excipients, (2006), 5th ed., which is hereby incorporated by reference in its entirety.

As used herein, the term “ion-exchange resin” refers to an ion-exchange resin that is pharmaceutically acceptable and that can be weakly acidic, weakly basic, strongly acidic or strongly basic. Suitable ion-exchange resins include, but are not limited to Amberlite™ IRP64, IRP88 and IRP69 (available from Rohm and Haas) and Duolite™ AP143 (available from Rohm and Haas). In some embodiments, the ion-exchange resin is a crosslinked polymer resin comprising acrylic acid, methacrylic acid, or polystyrene sulfonate, or salts thereof. In some embodiments, the ion-exchange resin is polacrilex resin, polacrilin potassium resin, or cholestyramine resin.

As used herein, the term “hydrogenated polyisobutene” (also known as liquid isoparaffin) refers to a hydrogenated polymer formed from isobutene and/or other comonomers. Suitable hydrogenated polyisobutenes include, but are not limited to, Sophim™ MC30 and MC300 (available from Sophim) and the Polyiso™ 200, 250, 275, 300, 450, and 800 polymers (available from The Fanning Corporation).

As used herein, the term “lauroyl macrogol glyceride” refers to a polyglycolized glyceride synthesized predominately from lauric acid or from compounds derived predominately from lauric acid, although other fatty acids or compounds derived from other fatty acids may used in the synthesis as well. Suitable lauroyl macrogol glycerides include, but are not limited to, Gelucire® 44/14 (available from Gattefossé).

As used herein, the term “lecithin” refers to a naturally occurring or synthetic lecithin, or phospholipid, which may be suitably refined. Suitable lecithins include, but are not limited to lecithins derived from egg or soy phosphatides, such as egg lecithin, egg phosphatidyl ethanolamine, phosphatidic acid, plant monogalactosyl diglycerides (hydrogenated) or plant digalactosyl diglyceride (hydrogenated) and the like. Other useful lecithins include, but are not limited to phosphatidylcholine and its derivatives, phosphatidylethanolamine and its derivatives, phosphatidylserine and its derivatives, or a polymeric lipid wherein a hydrophilic polymer is conjugated to the lipid headgroup. Further suitable lecithins include, but are not limited to dihexanoyl-L-alpha-lecithin, dioctanoyl-L-alpha-lecithin, didecanoyl-L-alpha-lecithin, didodecanoyl-L-alpha-lecithin, ditetradecanoyl-L-alpha-lecithin, dihexadecanoyl-L-alpha-lecithin, dioctadecanoyl-L-alpha-lecithin, dioleoyl-L-alpha-lecithin, dilinoleoyl-L-alpha-lecithin, alpha-palmito, beta-oleoyl-L-alpha-lecithin, L-alpha-glycerophosphoryl choline and the like. Commercially available lecithins useful in the present invention include, but are not limited to LSC 5050 and 6040 (available from Avatar Corp.), Phosal™ 50 PG and 53 MCT (available from American Lecithin, Inc.), Phospholipon™ 100H, 90G, 90H and 80 (available from American Lecithin, Inc.), sunflower based lecithins, Lecistar™ Sun 100 and 200 (available from SternChemie), soybean based lecithins, Greencithin™ (available from SternChemie), and soy based lecithins, Yellothin™ (available from SternChemie), as well as those listed in R. C. Rowe and P. J. Shesky, Handbook of pharmaceutical excipients, (2006), 5th ed., which is hereby incorporated by reference in its entirety.

As used herein, the term “linoleoyl macrogolglyceride” refers to a polyglycolized glyceride synthesized predominately from linoleic acid or from compounds derived predominately from linoleic acid, although other fatty acids or compounds derived from other fatty acids may used in the synthesis as well. Suitable linoleoyl macrogolglycerides include, but are not limited to, Labrafil™ M 2125 CS (available from Gattefossé).

Suitable mannitols include, but are not limited to, PharmMannidex (available from Cargill), Pearlitol (available from Roquette), and Mannogem (available from SPI Polyols).

As used herein, the term “metallic alkyl sulfate” refers to a metallic salt formed between inorganic base and an alkyl sulfate compound. In some embodiments, the metallic alkyl sulfate has about eight carbons to about eighteen carbons. In some embodiments, metallic alkyl sulfate is a metallic lauryl sulfate. In some embodiments, the metallic alkyl sulfate is sodium lauryl sulfate.

As used herein, the term “metal aluminosilicate” refers to any metal salt of an aluminosilicate, including, but not limited to, magnesium aluminometasilicate. Suitable magnesium aluminosilicates include, but are not limited to Neusilin (available from Fuji Chemical), Pharmsorb (available from Engelhard), and Veegum (available from R.T. Vanderbilt Co., Inc.). In some embodiments, the metal aluminosilicate is bentonite. In some embodiments, the metal aluminosilicate is kaolin.

As used herein, the term “metal carbonate” refers to any metallic carbonate, including, but not limited to sodium carbonate, calcium carbonate, and magnesium carbonate, and zinc carbonate.

As used herein, the term “metal oxide” refers to any metallic oxide, including, but not limited to, calcium oxide or magnesium oxide.

As used herein, the term “metallic stearate” refers to a metal salt of stearic acid. In some embodiments, the metallic stearate is calcium stearate, zinc stearate, or magnesium stearate. In some embodiments, the metallic stearate is magnesium stearate.

As used herein, the term “mineral oil” refers to both unrefined and refined (light) mineral oil. Suitable mineral oils include, but are not limited to, the Avatech™ grades (available from Avatar Corp.), Drakeol™ grades (available from Penreco), Sirius™ grades (available from Shell), and the Citation™ grades (available from Avater Corp.).

As used herein, the term “oleoyl macrogol glycerides” refers to a polyglycolized glyceride synthesized predominately from oleic acid or from compounds derived predominately from oleic acid, although other fatty acids or compounds derived from other fatty acids may used in the synthesis as well. Suitable oleoyl macrogol glycerides include, but are not limited to, Labrafil™ M 1944 CS (available from Gattefossé).

As used herein, the term “polyalkylene glycol”, employed alone or in combination with other terms, refers to a polymer containing oxyalkylene monomer units, or copolymer of different oxyalkylene monomer units. As used herein, the term “oxyalkylene”, employed alone or in combination with other terms, refers to a group of formula —O-alkylene-. In some embodiments, the polyalkylene glycol is polytetrahydrofuran. In some embodiments, the polyalkylene glycol is polybutylene glycol.

As used herein, the term “alkyl”, employed alone or in combination with other terms, refers to a saturated hydrocarbon group that may be straight-chain or branched. In some embodiments, the alkyl group contains 1 to 6 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, n-heptyl, n-octyl, and the like.

As used herein, the term “alkylene”, employed alone or in combination with other terms, refers to a divalent alkyl linking group. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like.

As used herein, the term “polyethylene glycol” refers to a polymer containing ethylene glycol monomer units of formula —O—CH₂—CH₂—. Suitable polyethylene glycols may have a free hydroxyl group at each end of the polymer molecule, or may have one or more hydroxyl groups etherified with a lower alkyl, e.g., a methyl group. Also suitable are derivatives of polyethylene glycols having esterifiable carboxy groups. Polyethylene glycols useful in the present invention can be polymers of any chain length or molecular weight, and can include branching. In some embodiments, the average molecular weight of the polyethylene glycol is from about 200 to about 9000. In some embodiments, the average molecular weight of the polyethylene glycol is from about 200 to about 5000. In some embodiments, the average molecular weight of the polyethylene glycol is from about 200 to about 900. In some embodiments, the average molecular weight of the polyethylene glycol is about 400. Suitable polyethylene glycols include, but are not limited to polyethylene glycol-200, polyethylene glycol-300, polyethylene glycol-400, polyethylene glycol-600, and polyethylene glycol-900. The number following the dash in the name refers to the average molecular weight of the polymer. In some embodiments, the polyethylene glycol is polyethylene glycol-400. Suitable polyethylene glycols include, but are not limited to the Carbowax™ and Carbowax™ Sentry series (available from Dow), the Lipoxol™ series (available from Brenntag), the Lutrol™ series (available from BASF), and the Pluriol™ series (available from BASF).

As used herein, the term “polyethoxylated fatty acid ester” refers to a monoester or diester, or mixture thereof, derived from the ethoxylation of a fatty acid. The polyethoyxylated fatty acid ester can contain free fatty acids and polyethylene glycol as well. Fatty acids useful for forming the polyethoxylated fatty acid esters include, but are not limited to, those described herein. Suitable polyethoxylated fatty acid esters include, but are not limited to, Emulphor™ VT-679 (stearic acid 8.3 mole ethoxylate, available from Stepan Products), the Alkasurf™ CO series (available from Alkaril), macrogol 15 hydroxystearate, Solutol™ HS15 (available from BASF), and the polyoxyethylene stearates listed in R. C. Rowe and P. J. Shesky, Handbook of pharmaceutical excipients, (2006), 5th ed., which is hereby incorporated by reference in its entirety.

As used herein, the term “polyethoxylated vegetable oil” refers to a compound, or mixture of compounds, formed from ethoxylation of vegetable oil, wherein at least one chain of polyethylene glycol is covalently bound to the vegetable oil. In some embodiments, the fatty acids has between about twelve carbons to about eighteen carbons. In some embodiments, the amount of ethoxylation can vary from about 2 to about 200, about 5 to 100, about 10 to about 80, about 20 to about 60, or about 12 to about 18 of ethylene glycol repeat units. The vegetable oil may be hydrogenated or unhydrogenated. Suitable polyethoxylated vegetable oils, include but are not limited to, Cremaphor™ EL or RH series (available from BASF), Emulphor™ EL-719 (available from Stepan products), and Emulphor™ EL-620P (available from GAF).

As used herein, the term “polyethoxylated castor oil”, refers to a compound formed from the ethoxylation of castor oil, wherein at least one chain of polyethylene glycol is covalently bound to the castor oil. The castor oil may be hydrogenated or unhydrogenated. Synonyms for polyethoxylated castor oil include, but are not limited to polyoxyl castor oil, hydrogenated polyoxyl castor oil, mcrogolglyceroli ricinoleas, macrogolglyceroli hydroxystearas, polyoxyl 35 castor oil, and polyoxyl 40 hydrogenated castor oil. Suitable polyethoxylated castor oils include, but are not limited to, the Nikkol™ HCO series (available from Nikko Chemicals Co. Ltd.), such as Nikkol HCO-30, HC-40, HC-50, and HC-60 (polyethylene glycol-30 hydrogenated castor oil, polyethylene glycol-40 hydrogenated castor oil, polyethylene glycol-50 hydrogenated castor oil, and polyethylene glycol-60 hydrogenated castor oil, Emulphor™ EL-719 (castor oil 40 mole-ethoxylate, available from Stepan Products), the Cremophore™ series (available from BASF), which includes Cremophore RH40, RH60, and EL35 (polyethylene glycol-40 hydrogenated castor oil, polyethylene glycol-60 hydrogenated castor oil, and polyethylene glycol-35 hydrogenated castor oil, respectively), and the Emulgin® RO and HRE series (available from Cognis PharmaLine). Other suitable polyoxyethylene castor oil derivatives include those listed in R. C. Rowe and P. J. Shesky, Handbook of pharmaceutical excipients, (2006), 5th ed., which is hereby incorporated by reference in its entirety.

As used herein, the term “polyethoxylated sterol” refers to a compound, or mixture of compounds, derived from the ethoxylation of a sterol molecule. Suitable polyethoyxlated sterols include, but are not limited to, PEG-24 cholesterol ether, Solulan™ C-24 (available from Amerchol); PEG-30 cholestanol, Nikkol™ DHC (available from Nikko); Phytosterol, GENEROL™ series (available from Henkel); PEG-25 phyto sterol, Nikkol™ BPSH-25 (available from Nikko); PEG-5 soya sterol, Nikkol™ BPS-5 (available from Nikko); PEG-10 soya sterol, Nikkol™ BPS-10 (available from Nikko); PEG-20 soya sterol, Nikkol™ BPS-20 (available from Nikko); and PEG-30 soya sterol, Nikkol™ BPS-30 (available from Nikko). As used herein, the term “PEG” refers to polyethylene glycol.

As used herein, the term “polyoxyethylene-glycerol fatty ester” refers to ethoxylated fatty acid ester of glycerine, or mixture thereof. In some embodiments, the polyoxyethylene portion of the molecule has about 2 to about 200 oxyethylene units. In some embodiments, the polyoxyethylene portion of the molecule has about 2 to about 100 oxyethylene units. In some embodiments, the polyoxyethylene portion of the molecule has about 4 to about 50 oxyethylene units. In some embodiments, the polyoxyethylene portion of the molecule has about 4 to about 30 oxyethylene units. Suitable polyoxyethylene-glycerol fatty esters include, but are not limited to, PEG-20 glyceryl laurate, Tagat™ L (Goldschmidt); PEG-30 glyceryl laurate, Tagat™ L2 (Goldschmidt); PEG-15 glyceryl laurate, Glycerox™ L series (Croda); PEG-40 glyceryl laurate, Glycerox™ L series (Croda); PEG-20 glyceryl stearate, Capmul™ EMG (ABITEC), Aldo MS-20 KFG (Lonza); PEG-20 glyceryl oleate, Tagat™ 0 (Goldschmidt); PEG-30 glyceryl oleate, Tagat™ 02 (Goldschmidt).

As used herein, the term, “polyethoxylated sorbitan ester” refers to a compound, or mixture thereof, derived from the ethoxylation of a sorbitan ester. Fatty acids useful for deriving the polyethoyxlated sorbitan esters include, but are not limited to, those described herein. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 4 to about 80 oxyethylene units. In some embodiments, the polyoxyethylene portion of the compound or mixture has about 4 to about 40 oxyethylene units. In some embodiments, the polyoxyethylene portion of the compound or mixture has about 4 to about 20 oxyethylene units. Suitable polyethoxylated sorbitan esters include, but are not limited to the Tween™ series (available from Uniqema), which includes Tween 20 (POE(20) sorbitan monolaurate), 21 (POE(4) sorbitan monolaurate), 40 (POE(20) sorbitan monopalmitate), 60 (POE(20) sorbitan monostearate), 60K (POE(20) sorbitan monostearate), 61 (POE(4) sorbitan monostearate), 65 (POE(20) sorbitan tristearate), 80 (POE(20) sorbitan monooleate), 80K (POE(20) sorbitan monooleate), 81 (POE(5) sorbitan monooleate), and 85 (POE(20) sorbitan trioleate). As used herein, the abbreviation “POE” refers to polyoxyethylene. The number following the POE abbreviation refers to the number of oxyethylene repeat units in the compound. Other suitable polyethoxylated sorbitan esters include the polyoxyethylene sorbitan fatty acid esters listed in R. C. Rowe and P. J. Shesky, Handbook of pharmaceutical excipients, (2006), 5th ed., which is hereby incorporated by reference in its entirety.

As used herein, the term “polyethoxylated cholesterol” refers to a compound, or mixture thereof, formed from the ethoxylation of cholesterol. In some embodiments, the polyoxyethylene portion of the compound or mixture has about 2 to about 200 oxyethylene units. In some embodiments, the polyoxyethylene portion of the compound or mixture has about 2 to about 100 oxyethylene units. In some embodiments, the polyoxyethylene portion of the compound or mixture has about 2 to about 50 oxyethylene units. In some embodiments, the polyoxyethylene portion of the compound or mixture has about 5 to about 30 oxyethylene units.

As used herein, the term “polyglycolized glycerides”, employed alone or in combination with other terms, refers to the products formed from the esterification of polyethylene glycol, glycerol, and fatty acids; the transesterification of glycerides and polyethylene glycol; or the ethoxylation of a glyceride of a fatty acid. As used herein, the term “polyglycolized glycerides” can, alternatively or additionally, refer to mixtures of monoglycerides, diglycerides, and/or triglycerides with monoesters and/or diesters of polyethylene glycol. Polyglycolized glycerides can be derived from the fatty acids, glycerides of fatty acids, and polyethylene glycols described herein. The fatty ester side-chains on the glycerides, monoesters, or diesters can be of any chain length and can be saturated or unsaturated. The polyglycolized glycerides can contain other materials as contaminants or side-products, such as, but not limited to, polyethylene glycol, glycerol, and fatty acids.

In some embodiments, the polyglycolized glyceride is lauroyl macrogol glycerides, stearoyl macrogol glycerides, linoleoyl macrogol glycerides, oleoyl macrogol glycerides, or caprylocaproyl macrogolglycerides.

As used herein, the term “polyoxyethylene-alkyl ether” refers to a monoalkyl or dialkylether of polyoxyethylene, or mixtures thereof. In some embodiments, the polyoxyethylene-alkyl ether is a polyoxyethylene fatty alcohol ether.

As used herein, the term “polyoxyethylene fatty alcohol ether” refers to an monoether or diether, or mixtures thereof, formed between polyethylene glycol and a fatty alcohol. Fatty alcohols that are useful for deriving polyoxyethylene fatty alcohol ethers include, but are not limited to, those defined herein. In some embodiments, the polyoxyethylene portion of the molecule has about 2 to about 200 oxyethylene units. In some embodiments, the polyoxyethylene portion of the molecule has about 2 to about 100 oxyethylene units. In some embodiments, the polyoxyethylene portion of the molecule has about 4 to about 50 oxyethylene units. In some embodiments, the polyoxyethylene portion of the molecule has about 4 to about 30 oxyethylene units. In some embodiments, the polyoxyethylene fatty alcohol ether comprises ethoxylated stearyl alcohols, cetyl alcohols, and cetylstearyl alcohols (cetearyl alcohols). Suitable polyoxyethylene fatty alcohol ethers include, but are not limited to, the Brij™ series of surfactants (available from Uniqema), which includes Brij 30, 35, 52, 56, 58, 72, 76, 78, 93Veg, 97, 98, and 721, the Cremophor™ A series (available from BASF), which includes Cremophor A6, A20, and A25, the Emulgen™ series (available from Kao Corp.), which includes Emulgen 104P, 123P, 210P, 220, 320P, and 409P, the Ethosperse™ (available from Lonza), which includes Ethosperse 1A4, 1A12, TDAa6, S120, and G26, the Ethylan™ series (available from Brenntag), which includes Ethylan D252, 253, 254, 256, 257, 2512, and 2560, the Plurafac™ series (available from BASF), which includes Plurafac RA20, RA30, RA40, RA43, and RA340, the Ritoleth™ and Ritox™ series (available from Rita Corp.), the Volpo™ series (available from Croda), which includes Volpo N 10, N 20, S2, S10, C2, C20, CS10, CS20, L4, and L23, and the Texafor™ series, which includes Texafor A1P, AP, A6, A10, A14, A30, A45, and A60. Other suitable polyoxyethylene fatty alcohol ethers include, but are not limited to, polyethylene glycol (13)stearyl ether (steareth-13), polyethylene glycol (14)stearyl ether (steareth-14), polyethylene glycol (15)stearyl ether (steareth-15), polyethylene glycol (16)stearyl ether (steareth-16), polyethylene glycol (17)stearyl ether (steareth-17), polyethylene glycol (18)stearyl ether (steareth-18), polyethylene glycol (19)stearyl ether (steareth-19), polyethylene glycol (20)stearyl ether (steareth-20), polyethylene glycol (12)isostearyl ether (isosteareth-12), polyethylene glycol (13)isostearyl ether (isosteareth-13), polyethylene glycol (14)isostearyl ether (isosteareth-14), polyethylene glycol (15)isostearyl ether (isosteareth-15), polyethylene glycol (16)isostearyl ether (isosteareth-16), polyethylene glycol (17)isostearyl ether (isosteareth-17), polyethylene glycol (18)isostearyl ether (isosteareth-18), polyethylene glycol (19)isostearyl ether (isosteareth-19), polyethylene glycol (20)isostearyl ether (isosteareth-20), polyethylene glycol (13)cetyl ether (ceteth-13), polyethylene glycol (14)cetyl ether (ceteth-14), polyethylene glycol (15)cetyl ether (ceteth-15), polyethylene glycol (16)cetyl ether (ceteth-16), polyethylene glycol (17)cetyl ether (ceteth-17), polyethylene glycol (18)cetyl ether (ceteth-18), polyethylene glycol (19)cetyl ether (ceteth-19), polyethylene glycol (20)cetyl ether (ceteth-20), polyethylene glycol (13)isocetyl ether (isoceteth-13), polyethylene glycol (14)isocetyl ether (isoceteth-14), polyethylene glycol (15)isocetyl ether (isoceteth-15), polyethylene glycol (16)isocetyl ether (isoceteth-16), polyethylene glycol (17)isocetyl ether (isoceteth-17), polyethylene glycol (18)isocetyl ether (isoceteth-18), polyethylene glycol (19)isocetyl ether (isoceteth-19), polyethylene glycol (20)isocetyl ether (isoceteth-20), polyethylene glycol (12)oleyl ether (oleth-12), polyethylene glycol (13)oleyl ether (oleth-13), polyethylene glycol (14)oleyl ether (oleth-14), polyethylene glycol (15)oleyl ether (oleth-15), polyethylene glycol (12)lauryl ether (laureth-12), polyethylene glycol (12)isolauryl ether (isolaureth-12), polyethylene glycol (13)cetylstearyl ether (ceteareth-13), polyethylene glycol (14)cetylstearyl ether (ceteareth-14), polyethylene glycol (15)cetylstearyl ether (ceteareth-15), polyethylene glycol (16)cetylstearyl ether (ceteareth-16), polyethylene glycol (17)cetylstearyl ether (ceteareth-17), polyethylene glycol (18)cetylstearyl ether (ceteareth-18), polyethylene glycol (19)cetylstearyl ether (ceteareth-19), and polyethylene glycol (20)cetylstearyl ether (ceteareth-20). The numbers following the “polyethylene glycol” term refer to the number of oxyethylene repeat units in the compound. Blends of polyoxyethylene fatty alcohol ethers with other materials are also useful in the invention. A non-limiting example of a suitable blend is Arlacel™ 165 or 165 VEG (available from Uniqema), a blend of glycerol monostearate with polyethylene glycol-100 stearate. Other suitable polyoxyethylene fatty alcohol ethers include those listed in R. C. Rowe and P. J. Shesky, Handbook of pharmaceutical excipients, (2006), 5th ed., which is hereby incorporated by reference in its entirety.

As used herein, the term “polyoxypropylene-glycerol fatty ester” refers to an propoxylated fatty acid ester of glycerine, or mixture thereof. Fatty acids useful for deriving the polyoxypropylene-glycerol fatty esters include, but are not limited to, those described herein. In some embodiments, the polyoxypropylene portion of the molecule has about 2 to about 200 oxyethylene units. In some embodiments, the polyoxyethylene portion of the molecule has about 2 to about 100 oxypropylene units. In some embodiments, the polyoxypropylene portion of the molecule has about 4 to about 50 oxypropylene units. In some embodiments, the polyoxypropylene portion of the molecule has about 4 to about 30 oxyethylene units.

As used herein, the term “polyglycerol fatty acid ester” refers to a compound, or mixture of compounds, derived from the esterification of a polyglycerol molecule with one or more fatty acids. In some embodiments, the polyglycerol portion of the compound or mixture is derived from about 2 to about 50, or about 2 to about 10, glycerol molecules. Fatty acids useful for deriving the polyglycerol fatty acid esters include, but are not limited to, those described herein. Suitable polyglycerol fatty acid esters include, but are not limited to, Tegosoft™ PC 31 and PC 41 (available from Goldschmidt) and Plurol™ Oleique CC497 (available from Gatefosse).

As used herein, the term “polyoxyethylene-polyoxyalkylene copolymer” refers to a copolymer that has both oxyethylene monomer units and oxyalkylene monomer units. Generally, these polymers can be formed from the ring-opening polymerization of ethylene oxide and an alkylene oxide monomer. Suitable oxyalkylene monomer units include, but are not limited to, oxypropylene and oxybutylene. The chain ends may have a free hydroxyl groups or may have one or more hydroxyl groups etherified with a lower alkyl or carboxy group. In some embodiments, the polyoxyethylene-polyoxyalkylene copolymer is a block copolymer, wherein one block is polyoxyethylene and the other block is polyoxyalkylene.

As used herein, the term “polyoxyethylene-polyoxypropylene copolymer” refers to a copolymer that has both oxyethylene monomer units and oxypropylene monomer units. Suitable polyoxyethylene-polyoxypropylene copolymers for use in the invention can be of any chain length or molecular weight, and can include branching. The chain ends may have a free hydroxyl groups or may have one or more hydroxyl groups etherified with a lower alkyl or carboxy group. The polyoxyethylene-polyoxypropylene copolymers can also include other monomers which were copolymerized and which form part of the backbone. For example, butylene oxide can be copolymerized with ethylene oxide and propylene oxide to form polyoxyethylene-polyoxypropylene copolymers useful in the present invention. In some embodiments, the polyoxyethylene-polyoxypropylene copolymer is a block copolymer, wherein one block is polyoxyethylene and the other block is polyoxypropylene. Suitable polyoxyethylene-polyoxypropylene copolymers include, but are not limited to, the Pluronic® series of surfactants (available from BASF), and which consist of the group of surfactants designated by the CTFA name of Poloxamer 108, 124, 188, 217, 237, 238, 288, 338, 407, 101, 105, 122, 123, 124, 181, 182, 183, 184, 212, 231, 282, 331, 401, 402, 185, 215, 234, 235, 284, 333, 334, 335, and 403. Other suitable polyoxyethylene-polyoxypropylene copolymers include, but are not limited to, DowFax® Nonionic surfactants (available from Dow Chemical), the DowFax® N-Series surfactants (available from Dow Chemical), Lutrol™ surfactants (available from BASF), and Synperonic™ surfactants (available from Uniqema).

As used herein, the term “polypropylene glycol” refers to a polymer containing propylene glycol monomer units of formula —O—C(CH₃)—CH₂—. The polypropylene glycols can be formed from the ring-opening polymerization of propylene oxide. Suitable polypropylene glycols for use in the invention can be of any chain length or molecular weight, and can include branching. The polypropylene glycols may have a free hydroxyl group at each end of the polymer molecule, or may have one or more hydroxyl groups etherified with a lower alkyl, e.g., a methyl group. Also suitable are derivatives of polypropylene glycols having esterifiable carboxy groups.

As used herein, the term “polyvinyl alcohol” refers to a polymer formed by partial or complete hydrolysis of polyvinyl acetate. Suitable polyvinyl alcohols include, but are not limited to, the Airvol series (available from Air Products), the Alcotex series (available from Synthomer), the Elvanol series (available from DuPont), the Gelvatol series (available from Burkard), and the Gohsenol series (available from Gohsenol).

As used herein, the term “polyvinylpyrrolidone” refers to a polymer of vinylpyrrolidone. In some embodiments, the polyvinylpyrrolidone contains one or more additional polymerized monomers. In some embodiments, the additional polymerized monomer is a carboxy containing monomer. In some embodiments, the polyvinylpyrrolidone is povidone. In some embodiments, the polyvinylpyrrolidone has a molecular weight between 2500 and 3 million. In some embodiments, the polyvinylpyrrolidone is povidone K12, K17, K25, K30, K60, K90, or K120. In some embodiments, the polyvinylpyrrolidone is povidone K25. Suitable polyvinylpyrrolidone polymers include, but are not limited to, the Kollidone™ series (available from BASF) and the Plasdone™ series (available from ISP).

As used herein, the term “propylene glycol fatty acid ester” refers to an monoether or diester, or mixtures thereof, formed between propylene glycol or polypropylene glycol and a fatty acid. Fatty acids that are useful for deriving propylene glycol fatty alcohol ethers include, but are not limited to, those defined herein. In some embodiments, the monoester or diester is derived from propylene glycol. In some embodiments, the monoester or diester has about 1 to about 200 oxypropylene units. In some embodiments, the polypropylene glycol portion of the molecule has about 2 to about 100 oxypropylene units. In some embodiments, the monoester or diester has about 4 to about 50 oxypropylene units. In some embodiments, the monoester or diester has about 4 to about 30 oxypropylene units. Suitable propylene glycol fatty acid esters include, but are not limited to, propylene glycol laurates: Lauroglycol™ FCC and 90 (available from Gattefosse); propylene glycol caprylates: Capryol™ PGMC and 90 (available from Gatefosse); and propylene glycol dicaprylocaprates: Labrafac™ PG (available from Gatefosse).

As used herein, the term “quaternary ammonium compound” refers a compound that contains at least one quaternary ammonium group. Particularly useful quaternary ammonium compound are those that are capable of emulsifying, solubilizing, or suspending hydrophobic materials in water. Alternatively, other useful quaternary ammonium compounds are those capable of stabilizing the semi-solid or liquid formulations during storage or processing. Other quaternary ammonium compounds useful in the invention are those that can enhance bioavailability of the active pharmacological agent when administered to the patient. Suitable quaternary ammonium compounds include, but are not limited to, 1,2-dioleyl-3-trimethylammonium propane, dimethyldioctadecylammonium bromide, N-[1-(1,2-dioleyloxy)propyl]-N,N,N-trimethylammonium chloride, 1,2-dioleyl-3-ethylphosphocholine, or 3-β-[N—[(N′,N′-dimethylamino)ethan]carbamoyl]cholesterol. Other suitable quaternary ammonium compounds include, but are not limited to, Stepanquat™ 5ONF and 65NF (n-alkyl dimethyl benzyl ammonium chloride, available from Stepan Products).

As used herein, the term “sorbitan ester” refers to a compound, or mixture of compounds, derived from the esterification of sorbitol and at least one fatty acid. Fatty acids useful for deriving the sorbitan esters include, but are not limited to, those described herein. Suitable sorbitan esters include, but are not limited to, the Span™ series (available from Uniqema), which includes Span 20 (sorbitan monolaurate), 40 (sorbitan monopalmitate), 60 (sorbitan monostearate), 65 (sorbitan tristearate), 80 (sorbitan monooleate), and 85 (sorbitan trioleate). Other suitable sorbitan esters include those listed in R. C. Rowe and P. J. Shesky, Handbook of pharmaceutical excipients, (2006), 5th ed., which is hereby incorporated by reference in its entirety.

Suitable sorbitols include, but are not limited to, Neosorb (available from Roquette), Partech™ SI (available from Merck), Liponic™ 70-NC and 76-NC (available from Lipo Chemical), and Sorbogem™ (available from SPI polyols).

Suitable squalenes include, but are not limited to, marine and olive squalenes (available from Sophim).

Starch, sodium starch glycolate, and pregelatinized starch include, but are not limited to, those described in R. C. Rowe and P. J. Shesky, Handbook of pharmaceutical excipients, (2006), 5th ed., which is hereby incorporated by reference in its entirety.

As used herein, the term “starch” refers to any type of natural or modified starch including, but not limited to, maize starch (also known as corn starch or maydis amylum), potato starch (also known as solani amylum), rice starch (also known as oryzae amylum), wheat starch (also known as tritici amylum), and tapioca starch. The term “starch” also refers to starches that have been modified with regard to molecular weight and branching. The term “starch” further refers to starches that have been chemically modified to attach chemical functionality such as carboxy, hydroxyl, hydroxyalkylene, or carboxyalkylene groups. As used herein, the term “carboxyalkylene” refers to a group of formula -alkylene-C(O)OH, or salt thereof. As used herein, the term “hydroxyalkylene” refers to a group of formula -alkylene-OH.

Suitable sodium starch glycolates include, but are not limited to, Explotab (available from JRS Pharma), Glycolys (available from Roquette), Primojel (available from DMV International), and Vivastar (available from JRS Pharma).

Suitable pregelatinized starches include, but are not limited to, Lycatab C and PGS (available from Roquette), Merigel (available from Brenntag), National 78-1551 (available from National Starch), Spress B820 (available from GPC), and Starch 1500 (available from Colorcon).

As used herein, the term “stearoyl macrogol glyceride” refers to a polyglycolized glyceride synthesized predominately from stearic acid or from compounds derived predominately from stearic acid, although other fatty acids or compounds derived from other fatty acids may used in the synthesis as well. Suitable stearoyl macrogol glycerides include, but are not limited to, Gelucire® 50/13 (available from Gattefossé).

As used herein, the term “sugar ester of fatty acid” refers to an ester compound formed between a fatty acid and carboxydrate or sugar molecule. In some embodiments, the carbohydrate is glucose, lactose, sucrose, dextrose, mannitol, xylitol, sorbitol, maltodextrin and the like. Suitable sugar esters of fatty acids include, but are not limited to sucrose fatty acid esters (such as those available from Mitsubishi Chemicals).

As used herein, the term “sulfosuccinate” refers to an dialkyl sulfosuccinate metal salt of formula, R—O—C(O)CH₂CH(SO₃⁻M⁺)C(O)O—R, wherein R is alkyl or cycloalkyl, wherein alkyl and cycloalkyl may be optionally substituted with one or more hydroxyl groups, and M is a metal, such as sodium, potassium and the like. In some embodiments, R is isobutyl, amyl, hexyl, cyclohexyl, octyl, tridecyl, or 2-ethylhexyl. Suitable sulfosuccinates are the Aerosol™ series of sulfosuccinate surfactants (available from Cytec).

As used herein, the term “taurate” refers to an alkyl taurate metal salt of formula, R—C(O)NR′—CH₂—CH₂—SO₃⁻M⁺, wherein R and R′ are alkyl or cycloalkyl, wherein alkyl and cycloalkyl may be optionally substituted with one or more hydroxyl groups, and M is a metal, such as sodium, potassium and the like. In some embodiments, R is cocoyl or oleyl. In some embodiments, R′ is methyl or ethyl. Suitable taurates include, but are not limited to, the Geropon™ T series, which includes Geropon™ TC 42 and T 77 (available from Rhodia) and the Hostapon™ T series (available from Clariant).

As used herein, the term “vegetable oil” refers to naturally occurring or synthetic oils, which may be refined, fractionated or hydrogenated, including triglycerides. Suitable vegetable oils include, but are not limited to castor oil, hydrogenated castor oil, sesame oil, corn oil, peanut oil, olive oil, sunflower oil, safflower oil, soybean oil, benzyl benzoate, sesame oil, cottonseed oil, and palm oil. Other suitable vegetable oils include commercially available synthetic oils such as, but not limited to, Miglyol™ 810 and 812 (available from Dynamit Nobel Chicals, Sweden) Neobee™ M5 (available from Drew Chemical Corp.), Alofine™ (available from Jarchem Industries), the Lubritab™ series (available from JRS Pharma), the Sterotex™ (available from Abitec Corp.), Softisan™ 154 (available from Sasol), Croduret™ (available from Croda), Fancol™ (available from the Fanning Corp.), Cutina™ HR (available from Cognis), Simulsol™ (available from CJ Petrow), EmCon™ CO (available from Amisol Co.), Lipvol™ CO, SES, and HS-K (available from Lipo), and Sterotex™ HM (available from Abitec Corp.). Other suitable vegetable oils, including sesame, castor, corn, and cottonseed oils, include those listed in R. C. Rowe and P. J. Shesky, Handbook of pharmaceutical excipients, (2006), 5th ed., which is hereby incorporated by reference in its entirety.

In the pharmaceutical ingredient definitions, one of skill in the art will recognize that certain formulation ingredients may fall into more than one classification of the definitions herein. For example, a sugar ester of fatty acid may also be regarded as a fatty acid ester.

As will be appreciated, some components of the pharmaceutical formulations of the invention can possess multiple functions. For example, a given component can act as both a carrier and a emulsifier/solubilizing agent. In some such cases, the function of a given component can be considered singular, even though its properties may allow multiple functionality.

### Preparation of the Active Pharmacological Agent

ERB-041, 2-(3-fluoro-4-hydroxyphenyl)-7-vinyl-1,3-benzoxazol-5-ol, can be made by the methods described in U.S. Pat. No. 6,794,403, hereby incorporated by reference in its entirety. The anhydrate crystal form of ERB-041 (Form B) used in the formulations of the present invention can be prepared by any of various suitable means, and can be distinguished from other anhydrate (anhydrous) crystal forms, hydrate crystal forms and/or amorphous form of ERB-041 by its unique solid state signature.

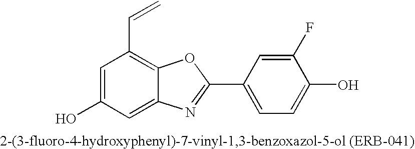

The anhydrate crystal form of ERB-041 (Form B) used in the formulations of the present invention can be prepared by any of various suitable means. For example, the anhydrate (Form B) can be prepared by precipitation from an anhydrous solution. An anhydrous solution is substantially free of water, i.e., containing less than about 4%, less than about 3%, less than about 2%, less than about 1%, less than about 0.5%, less than about 0.2%, less than about 0.1%, less than about 0.05%, or less than 0.01% by volume of water. In some embodiments, the solution is prepared in a suitable solvent near saturation. Suitable solvents for precipitating the anhydrate crystal form include polar aprotic organic solvents such as ketones (e.g., acetone or the like), organic nitrile (e.g., acetonitrile or the like), and mixture thereof. In some embodiments, the anhydrate is precipitated from a solvent containing acetonitrile or acetone. In some embodiments, the anhydrate is precipitated from a solvent containing acetonitrile. In some embodiments, the anhydrate is precipitated from a solvent containing acetone.

Precipitation of the anhydrate (Form B) can be induced by any of the various well known methods of precipitation. For example, precipitation can be induced by cooling the solution or evaporation of solvents (optionally under reduced pressure). In some embodiments, the solution is cooled from a temperature of about 40° C. to about 100° C., about 50° C. to about 90° C., about 50° C. to about 80° C., or about 50° C. to about 70° C. down to a temperature of about −20° C. to about 30° C., about 20° C. to about 30° C. (room temperature), about 0° C. to about 10° C., or about 0° C. to about 5° C. During the cooling process, the temperature can be optionally held at an intermediate temperature such as about 70° C., or about 40° C. to about 60° C. (e.g., about 45° C. to about 50° C.) for a period of time. In some embodiments, the solution is cooled from a temperature close to the boiling point of the selected solvent. In some embodiment, the solution is cooled from about 70° C. when the boiling point of the selected solvent is above about 70° C. In some embodiment, the solution is cooled from about 50° C. when the boiling point of the selected solvent is above about 50° C. In some embodiments, the solution is cooled to room temperature. In some embodiments, the solution is cooled without a cold bath. In some embodiments, the solution is cooled with a cold bath.

The rate of the cooling can be adjusted to facilitate the precipitation of the anhydrate crystal form (Form B) of ERB-041. In some embodiments, the anhydrate crystal form is precipitated from an acetone solution of ERB-041 by fast cooling. In some embodiments, the anhydrate crystal form is precipitated from an acetonitrile solution of ERB-041 by slow cooling.

As used herein, “fast cooling” refers to cooling of the solution from a temperature of about 40° C. to about 100° C., about 50° C. to about 90° C., about 50° C. to about 80° C., or about 50° C. to about 70° C. down to a temperature of about −20° C. to about 30° C., about 20° C. to about 30° C. (room temperature), about 0° C. to about 10° C., or about 0° C. to about 5° C. in less than about 70 minutes, about 60 minutes, about 50 minutes, about 40 minutes, about 30 minutes, about 20 minutes, about 15 minutes, about 10 minutes, about 5 minutes, about 2 minutes, or about 1 minute. In some embodiments, fast cooling is carried out to cool the solution from a temperature of about 50 to about 70, about 50, or about 70° C. to room temperature (about 20° C. to about 30° C.) in less than about 30 minutes, about 20 minutes, about 15 minutes, about 10 minutes, about 5 minutes, about 2 minutes, or about 1 minute. In some embodiments, fast cooling is carried out in between about 2 minutes and about 10 minutes.

As used herein, “slow cooling” refers to cooling of the solution from about a temperature of about 40° C. to about 100° C., about 50° C. to about 90° C., about 50° C. to about 80° C., or about 50° C. to about 70° C. down to a temperature of about −20° C. to about 30° C., about 20° C. to about 30° C. (room temperature), about 0° C. to about 10° C., or about 0° C. to about 5° C. in more than about 1.5 hours, about 1.8 hours, about 2.0 hours, about 2.5 hours, or about 3.0 hours. In some embodiments, slow cooling is carried out to cool the solution from a temperature of about 50° C. to about 70° C., about 50° C., or about 70° C. to room temperature (about 20° C. to about 30° C.) in more than about 2.0 hours, about 2.5 hours, or about 3.0 hours. In some embodiments, slow cooling is carried out in between about 2 hours and about 3.0 hours.

After the solution is cooled (optionally with stirring), precipitation occurs and a suspension forms. The suspension can optionally be stirred at the cooled temperature (e.g., room temperature) for an additional period of time (such as 1 hour, 2 hours, 12 hours, 24 hours or longer). The solid is then collected, for example by filtering the suspension, and the solid (the crystal form: Form B) is dried optionally under reduced pressure (e.g., in a vacuum oven) optionally at an elevated temperature (i.e., a temperature above room temperature), preferably at about 50° C.

Alternatively, the anhydrate crystal form (Form B) can be precipitated from an acetonitrile solution of ERB-041 by fast evaporation of the solvents (optionally under reduced pressure) at an elevated temperature (e.g., at about 50° C. or about 70° C.). Preferably, the solution is heated to maintain the temperature so precipitation is not caused by cooling due to evaporation. In some embodiments, an acetonitrile solution of ERB-041 is placed in a vacuum oven at 50° C. and high vacuum is applied to remove the solvents. In some embodiments, the precipitation occurs under vacuum in less than about 40 minutes, about 30 minutes, about 20 minutes, about 15 minutes, about 10 minutes, about 5 minutes, about 2 minutes, or about 1 minute.

After the precipitation occurs and a suspension forms, the solid is collected, for example by filtering the suspension, and the solid (the crystal form: Form B) is dried optionally under reduced pressure (e.g., in a vacuum oven) optionally at an elevated temperature (e.g., at about 50° C.).

As used herein, “fast evaporation” refers to evaporation of solvents in a solution at an elevated temperature (e.g., at about 50° C. or about 70° C.) optionally facilitated by reduced pressure, thereby the resulting precipitation occurs in less than about 40 minutes, about 30 minutes, about 20 minutes, about 15 minutes, about 10 minutes, about 5 minutes, about 2 minutes, or about 1 minute. In some embodiments of the fast evaporation, the solvent in the solution evaporates about 10%, about 20%, about 30%, about 40%, about 50%, about 60%, or about 70% by volume in less than about 40 minutes, about 30 minutes, about 20 minutes, about 15 minutes, about 10 minutes, about 5 minutes, about 2 minutes, or about 1 minute.

The andrate crystal form (Form B) of ERB-041 can be identified by its unique solid state signatures with respect to, for example, differential scanning calorimetry (DSC), X-ray powder diffraction (XRPD), and other solid state methods. Further characterization with respect to water or solvent content of the crystal forms can be gauged by any of various routine methods such as thermogravimetric analysis (TGA) DSC and other techniques. For DSC, it is known that the temperatures observed will depend upon the rate of temperature change as well as sample preparation technique and the particular instrument employed. Thus, the values reported herein relating to DSC thermograms can vary by plus or minus about 4° C. Accordingly, the term “about” as used in connection with a given DSC temperature value, is intended to mean plus or minus 4° C. For XRPD, the relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus about 0.2°. Accordingly, the term “about” as used in connection with a given 2-theta value, is intended to mean plus or minus 0.2°.

The physical properties and X-ray data distinguishing the anhydrate crystal form (Form B) of ERB-041 are summarized in Tables 1 and 2 below.

TABLE 1
Physical properties of the anhydrate crystal form (Form B)

| | |
|---|---|
| TGA | less than about 0.2% |
| DSC | Melt onset ~246° C. |
| XRPD | 8.5, 10.3, 15.3° 2θ |

TABLE 2
X-ray data of the anhydrate crystal form (Form B)

| Peak position, 2θ° | Peak Description |
|---|---|
| 7.7 | W |
| 8.5 | S |
| 9.8 | M |
| 10.8 | S |
| 13.8 | M |
| 14.2 | W |
| 14.6 | VW |
| 15.3 | Strongest |
| 15.8 | M |
| 17.2 | W |
| 19.2 | M |
| 20.6 | VW |
| 22.3 | VW |
| 23.3 | M |
| 23.7 | W |
| 24.5 | VW |
| 25.8 | W |
| 27.2 | W |
| 27.9 | VW |
| 29.1 | VW |
| 30.2 | VW |
| 31.1 | VW |

S: relatively high peak intensity
M: middle range peak intensity
W: relatively weak peak intensity
VW: very weak peak intensity

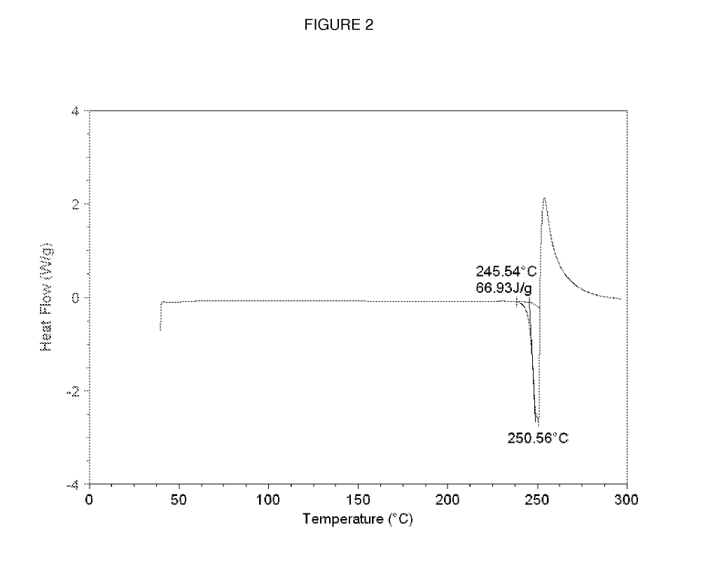

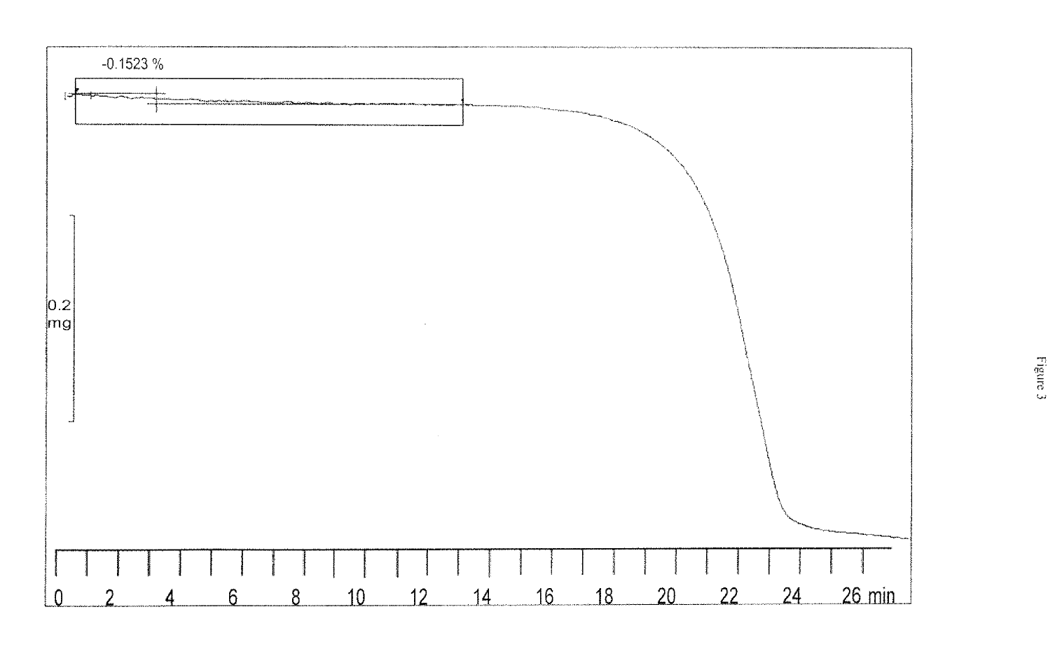

As can be seen from Table 1, the anhydrate (anhydrous) crystal form (Form B) has essentially no water content, showing a weight loss of less than about 0.2% by TGA (see also FIG. 3) and a lack of a dehydration endotherm in the DSC (see also FIG. 2).

In accordance with the distinguishing features provided by DSC and TGA analysis, the anhydrate (anhydrous) crystal form (Form B) of ERB-041 has a differential scanning calorimetry trace comprising a melting endotherm having an onset at about 246° C. and substantially lacking an endotherm corresponding to a dehydration event. In some embodiments, the crystal form has a differential scanning calorimetry trace substantially as shown in FIG. 2. In further embodiments, the crystal form can have a thermogravimetric analysis profile showing less than about 1%, less than about 0.8%, less than about 0.6%, less than about 0.5%, less than about 0.4%, less than about 0.3%, less than about 0.2%, less than about 0.1%, or less than about 0.05% weight loss from about 60 to about 150° C. In yet further embodiments, the crystal form can have a have a thermogravimetric analysis profile substantially as shown in FIG. 3.

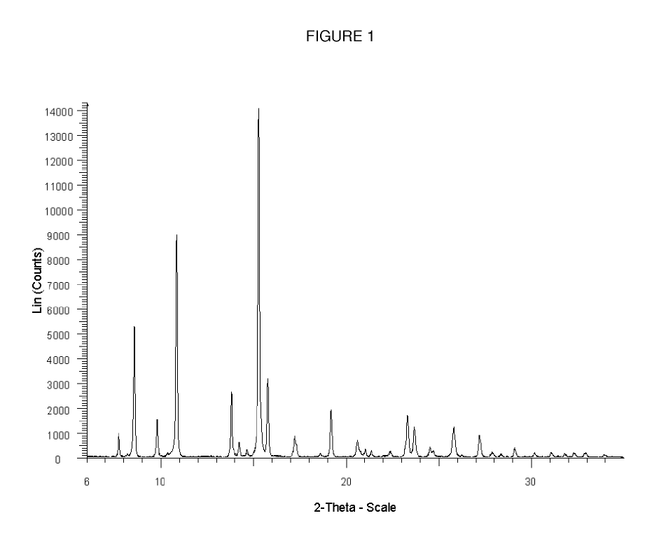

The crystal form (Form B) has a distinct XRPD pattern (see, e.g., FIG. 1), allowing characterization thereof based on the unique spectral signature. Accordingly, the anhydrous crystal form has an X-ray powder diffraction pattern comprising peaks, in terms of 2θ, at about 8.5°, about 10.8°, and about 15.3°. In some embodiments, the crystal form has an X-ray powder diffraction pattern comprising peaks, in terms of 2θ, at about 8.5°, about 10.8°, about 13.8°, about 15.3°, and about 15.8°. In some further embodiments, the crystal form has an X-ray powder diffraction pattern further comprising at least one peak, in terms of 2θ, selected from at about 9.8°, about 19.2°, and about 23.3°. In some embodiments, the crystal form has an X-ray powder diffraction pattern comprising peaks, in terms of 2θ, at about 8.5°, about 9.8°, about 10.8°, about 13.8°, about 15.3°, about 15.8°, about 19.2°, and about 23.3°. In further embodiments, the crystal form has an X-ray powder diffraction pattern substantially as shown in FIG. 1.

The anhydrate crystal form (Form B) can be converted to other crystal forms under suitable conditions. For example, after the anhydrate crystal form is suspended and stirred in water at room temperature for three days, a hydrate crystal form is obtained (the hydrate crystal form has been disclosed in U.S. patent application Ser. No. 11/369,405, filed Mar. 6, 2006). For another example, after the anhydrate crystal form is suspended and stirred in methanol at room temperature for one day, another anhydrate crystal form is obtained (the other anhydrate crystal form has been disclosed in U.S. provisional patent application Ser. No. 60/860,246 filed Nov. 21, 2006, which is hereby incorporated by reference in its entirety). For yet another example, after the anhydrate crystal form is suspended and stirred in ethanol at room temperature for three days, another anhydrate crystal form is obtained {the other anhydrate crystal form has been disclosed in U.S. patent application Ser. No. 11/369,405, filed Mar. 6, 2006).

### Administration and Preparation of the Pharmaceutical Formulations and Compositions

In general, the anhydrate crystal form (Form B) in the pharmaceutical formulations of the invention is present in a pharmaceutically effective amount. The phrase “pharmaceutically effective amount” refers to the amount of a compound {such as ERB-041 (the crystal form, Form B, of ERB-041)} that elicits the biological or medicinal response in a tissue, system, animal, individual, patient, or human that is being sought by a researcher, veterinarian, medical doctor or other clinician. The desired biological or medicinal response may include preventing the disorder in a patient (e.g., preventing the disorder in a patient that may be predisposed to the disorder, but does not yet experience or display the pathology or symptomatology of the disease). The desired biological or medicinal response may also include inhibiting the disorder in a patient that is experiencing or displaying the pathology or symptomatology of the disorder (i.e., arresting or slowing further development of the pathology and/or symptomatology). The desired biological or medicinal response may also include ameliorating the disorder in a patient that is experiencing or displaying the pathology or symptomatology of the disease (i.e., reversing the pathology or symptomatology).

The pharmaceutically effective amount provided in the propylaxis or treatment of a specific disorder may vary according to the specific condition(s) being treated, the size, age and response pattern of the patient, the severity of the disorder, the judgment of the attending physician or the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In general, the pharmaceutical formulations, and compositions thereof, can be administered by any appropriate route, for example, orally, parenterally, intravenously, intradermally, transdermally, or topically, in liquid, semi-solid or solid form. Parenteral administration includes intravenous, intraarterial, subcutaneous, intraperitoneal or intramuscular injection or infusion; or intracranial, e.g., intrathecal or intraventricular, administration. Parenteral administration can be in the form of a single bolus dose, or may be, for example, by a continuous perfusion pump. The preferred mode of administration is oral.

For example, where the liquid or semi-solid pharmaceutical formulations of the invention are sterile suspensions, they are suitable for intramuscular, intraperitoneal or subcutaneous injection. For another example, the liquid or semi-solid pharmaceutical formulations and the solid pharmaceutical formulations of the invention are suitable for oral administration.

The liquid or semi-solid pharmaceutical formulations of the invention can be administered rectally or vaginally in the form of a conventional suppository. For administration by intranasal or intrabronchial inhalation or insufflation, the crystal form (Form B) of ERB-041 can be formulated into an aqueous or partially aqueous solution, which can then be utilized in the form of an aerosol. The liquid or semi-solid formulations of the invention, and compositions thereof, can also be administered transdermally through the use of a transdermal patch allowing delivery of the agent for systemic absorption into the blood stream via the skin.

The liquid, semi-solid and the solid pharmaceutical formulations of the invention can comprise any conventionally used oral forms, including tablets, capsules, buccal forms, troches, lozenges and oral liquids, suspensions, and the like. Capsules or tablets containing the present pharmaceutical formulations can also be combined with mixtures of other active compounds or inert fillers and/or diluents. Oral pharmaceutical formulations used herein may utilize standard delay or time release formulations or spansules.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The compositions and formulations herein may also be combined and processed as a solid, then placed in a capsule form such as a gelatin capsule.

The liquid, semi-solid and the solid pharmaceutical formulations herein can also contain an antioxidant or a mixture of antioxidants such as ascorbic acid. Other antioxidants that can be used include sodium ascorbate and ascorbyl palmitate, optionally in conjunction with an amount of ascorbic acid. An example range for the antioxidant(s) is from about 0.05% to about 15% by weight, from about 0.5% to about 15% by weight, or from about 0.5% to about 5% by weight. In some embodiments, the pharmaceutical formulations contain substantially no antioxidant.

Additional numerous various excipients, dosage forms, dispersing agents and the like that are suitable for use in connection with the pharmaceutical formulations of the invention are known in the art and described in, for example, Remington's Pharmaceutical Sciences, 17th ed., Mack Publishing Company, Easton, Pa., 1985, which is hereby incorporated by reference in its entirety.

In order that the invention disclosed herein may be more efficiently understood, examples are provided below. It should be understood that these examples are for illustrative purposes only and are not to be construed as limiting the invention in any manner.

### EXAMPLES

As used herein, the term “Cₘₐₓ” refers to the maximum concentration of the active pharmacological agent in the blood plasma in the patient reached after dosing. As used herein, the term “tₘₐₓ” refers to the time it takes for the active pharmacological agent to reach its maximum concentration in the blood plasma of the patient after dosing. As used herein, the term “t_{1/2}” refers to plasma half-life, or the time it takes for the concentration of the active pharmacological agent in the blood plasma of the patient to decrease to half of Cₘₐₓ.

As used herein, the term “AUC” refers to the area under the plasma drug concentration as a function of time curve. As used herein, the term “AUCₜ” refers to the area under the plasma drug concentration curve up to a time point “t”. As used herein, the term, “AUC_{0→∞}” refers to the area under the whole curve up to infinite time.

### Example 1

### Preparation of an Anhydrate Crystal Form: Form B (Preparation 1)

Solid 2-(3-fluoro-4-hydroxyphenyl)-7-vinyl-1,3-benzoxazol-5-ol (0.22 g) was dissolved in acetone (2 ml) at 50° C. The solution was then cooled to room temperature with stirring in about 10 minutes during which time the solids precipitated. The suspension was filtered off, and the solid obtained was dried at 45-55° C., 5-10 mm Hg, to give the target crystalline/crystal form: Form B.

### Example 2

### Preparation of an Anhydrate Crystal Form: Form B (Preparations 2 and 3)

### Preparation 2

Solid 2-(3-fluoro-4-hydroxyphenyl)-7-vinyl-1,3-benzoxazol-5-ol (1 g) was added in acetonitrile (3 ml) at about 70° C. and stirred for 24 hrs. The suspension was then filtered. The filtrate was then slowly cooled to room temperature with stirring in about 2.5 hours during which time the solids precipitated. The solid formed was filtered off and dried at 45-55° C., 5-10 mm Hg, to give the target crystalline/crystal form: Form B.

### Preparation 3

Solid 2-(3-fluoro-4-hydroxyphenyl)-7-vinyl-1,3-benzoxazol-5-ol (1 g) was added in acetonitrile (3 ml) at about 70° C. and stirred for 24 hrs. The suspension was then filtered. The filtrate was then cooled to about 50° C. and placed in a vacuum oven at about 50° C. A high vacuum (5-10 mm Hg) was applied to the oven to remove the solvent for about 4 hrs to generate the target crystalline/crystal form: Form B.

### Example 3

### Acquisition of X-Ray Powder Diffraction Data of Form B

X-Ray data (e.g., see FIG. 1 and Table 2) was acquired using an X-ray powder diffractometer (Bruker-axs, model D8 advance) having the following parameters: voltage 40 kV, current 40.0 mA, scan range (2θ) 6 to 35°, total scan time 29 minutes, no Ni filter, detector slit 0.2 mm, and antiscattering slit 1 mm.

### Example 4

### Acquisition of Differential Scanning Calorimetry Data of Form B

Differential scanning calorimetry data (see FIG. 2) was collected using a DSC (TA instrument, model Q1000) under the following parameters: 50 mL/min purge gas (N₂), scan range 37 to 300° C., scan rate 10° C./min.

### Example 5

### Acquisition of Thermogravimetric Analysis Data of Form B

Thermogravimetric analysis data (see FIG. 3) was collected using a TGA instrument (Mettler Toledo, model TGA/SDTA 851e) under the following parameters: 40 mL/min purge gas (N₂); scan range 30 to 300° C., scan rate 20° C./min.

### Example 6

### A Liquid Formulation of Form B of 2-(3-Fluoro-4-Hydroxyphenyl)-7-Vinyl-1,3-Benzoxazol-5-Ol

The liquid formulation is prepared by the following procedure using the active ingredients in the percentages shown in Table 3.

1. Each of the active ingredients is weighed out independently.

2. The polyethylene glycol is placed in a mixer bowel and mixing begins.

3. The polyoxyethylene 20 sorbitan monooleate (Tween 80) and polyvinylpyrrolidone (povidone K25) are added to the mixer bowel and mixed.

4. Form B of 2-(3-fluoro-4-hydroxyphenyl)-7-vinyl-1,3-benzoxazol-5-ol is added to the mixture of step 3 and mixed until the crystal form (Form B) is dissolved/dispersed/suspended.

TABLE 3

| INGREDIENT | % WT/WT |
|---|---|
| Form B of 2-(3-fluoro-4- | 7.5 |
| hydroxyphenyl)-7-vinyl-1,3- | |
| benzoxazol-5-ol | |
| Polyethylene glycol 400 | 81.5 |
| Polyoxyethylene 20 Sorbitan | 1.0 |
| Monooleate (Tween 80) | |
| Polyvinylpyrrolidone (povidone | 10 |
| K25) | |

### Example 7

### Soft Gel Capsule Containing the Liquid Formulation of Form B of 2-(3-Fluoro-4-Hydroxyphenyl)-7-Vinyl-1,3-Benzoxazol-5-Ol

The liquid formulation of Example 6 is then poured into a soft gelatin capsule and sealed such that each capsule contains 75 mg of Form B of 2-(3-fluoro-4-hydroxyphenyl)-7-vinyl-1,3-benzoxazol-5-ol.

### Example 8

### A Semi-Solid Formulation of Form B of 2-(3-Fluoro-4-Hydroxyphenyl)-7-Vinyl-1,3-Benzoxazol-5-Ol

The semi-solid formulation is prepared by the following procedure using the active ingredients in the percentages shown in Table 4.

1. Each of the active ingredients is weighed out independently.

2. The Gelucire 44/14 is placed in a mixer bowel and mixing begins.

3. The polyoxyethylene 20 sorbitan monooleate (Tween 80) and polyvinylpyrrolidone (povidone K25) is added to the mixture of step 2 and mixed.

4. Form B of 2-(3-fluoro-4-hydroxyphenyl)-7-vinyl-1,3-benzoxazol-5-ol is added to the mixture of step 3 and is mixed to suspend the crystal form (Form B).

TABLE 4

| INGREDIENT | % WT/WT |
|---|---|
| Form B of 2-(3-fluoro-4- | 15 |
| hydroxyphenyl)-7-vinyl-1,3- | |
| benzoxazol-5-ol | |
| Gelucire 44/14 | 75 |
| Polyoxyethylene 20 Sorbitan | 5 |
| Monooleate (Tween 80) | |
| Polyvinylpyrrolidone (povidone | 5 |
| K25) | |

### Example 9

### Hard Gel Capsule Containing the Semi-Solid Formulation of Form B of 2-(3-Fluoro-4-Hydroxyphenyl)-7-Vinyl-1,3-Benzoxazol-5-Ol

While still warm, the semi-solid formulation of Example 8 is then poured into a hard gelatin capsule such that each capsule contains 75 mg of Form B of 2-(3-fluoro-4-hydroxyphenyl)-7-vinyl-1,3-benzoxazol-5-ol. The semi-solid formulation is continually mixed prior to pouring the semi-solid formulation into the capsule to maintain an even drug dispersion in the formulation. After pouring, the capsules are allowed to cool to room temperature to form a semi-solid mass.

### Example 10

### A Semi-Solid Formulation of Form B of 2-(3-Fluoro-4-Hydroxyphenyl)-7-Vinyl-1,3-Benzoxazol-5-Ol

The semi-solid formulation is prepared by the following procedure using the active ingredients in the percentages shown in Table 5.

1. Each of the active ingredients is weighed out independently.

2. The Gelucire 44/14 is placed in a mixer bowel that is then heated to 50 to 80° C. to melt the Gelucire 44/14.

3. The Labrasol, polyoxyethylene 20 sorbitan monooleate (Tween 80) and polyvinylpyrrolidone (povidone K25) are added to the mixture of step 2 and mixed.

4. Form B of 2-(3-fluoro-4-hydroxyphenyl)-7-vinyl-1,3-benzoxazol-5-ol is added to the mixture of step 3 and is mixed to suspend the crystal form (Form B).

TABLE 5

| INGREDIENT | % WT/WT |
|---|---|
| Form B of 2-(3-fluoro-4- | 15 |
| hydroxyphenyl)-7-vinyl-1,3- | |
| benzoxazol-5-ol | |
| Gelucire 44/14 | 40 |
| Labrasol | 35 |
| Polyoxyethylene 20 Sorbitan | 5 |
| Monooleate (Tween 80) | |
| Polyvinylpyrrolidone (povidone | 5 |
| K25) | |

### Example 11

### Hard Gel Capsule Containing the Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 10.

### Example 12

A Semi-Solid Formulation of Form B of 2-(3-Fluoro-4-Hydroxyphenyl)-7-Vinyl-1,3-Benzoxazol-5-Ol

The semi-solid formulation is prepared by the procedure of Example 10 using the active ingredients in the percentages shown in Table 6.

TABLE 6

| INGREDIENT | % WT/WT |
|---|---|
| Form B of 2-(3-fluoro-4- | 15 |
| hydroxyphenyl)-7-vinyl-1,3- | |
| benzoxazol-5-ol | |
| Gelucire 44/14 | 15 |
| Labrasol | 60 |
| Polyoxyethylene 20 Sorbitan | 5 |
| Monooleate (Tween 80) | |
| Polyvinylpyrrolidone (povidone | 5 |
| K25) | |

### Example 13

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 12.

### Example 14

### A Semi-Solid Formulation of Form B of 2-(3-Fluoro-4-Hydroxyphenyl)-7-Vinyl-1,3-Benzoxazol-5-Ol

The semi-solid formulation is prepared by the following procedure using the active ingredients in the percentages shown in Table 7.

1. Each of the active ingredients are weighed out independently.

2. The Gelucire 44/14 is placed in a mixer bowel that is then heated to 50 to 80° C. to melt the Gelucire 44/14.

3. The Labrasol and polyoxyethylene 20 sorbitan monooleate (Tween 80) are added to the mixture of step 2 and mixed.

4. Form B of 2-(3-fluoro-4-hydroxyphenyl)-7-vinyl-1,3-benzoxazol-5-ol is added to the mixture of step 3 and is mixed to suspend the crystal form (Form B).

TABLE 7

| INGREDIENT | % WT/WT |
|---|---|
| Form B of 2-(3-fluoro-4- | 15 |
| hydroxyphenyl)-7-vinyl-1,3- | |
| benzoxazol-5-ol | |
| Gelucire 44/14 | 40 |
| Labrasol | 40 |
| Polyoxyethylene 20 Sorbitan | 5 |
| Monooleate (Tween 80) | |

### Example 15

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 14.

### Example 16

### A Semi-Solid Formulation of Form B of 2-(3-Fluoro-4-Hydroxyphenyl)-7-Vinyl-1,3-Benzoxazol-5-Ol

The semi-solid formulation is prepared by the following procedure using the active ingredients in the percentages shown in Table 8.

1. Each of the active ingredients is weighed out independently.

2. The Gelucire 44/14 is placed in a mixer bowel that is then heated to 50 to 80° C. to melt the Gelucire 44/14.

3. The Labrasol and polyvinylpyrrolidone (povidone K25) are added to the mixture of step 2 and mixed.

4. Form B of 2-(3-fluoro-4-hydroxyphenyl)-7-vinyl-1,3-benzoxazol-5-ol is added to the mixture of step 3 and is mixed to suspend the crystal form (Form B).

TABLE 8

| INGREDIENT | % WT/WT |
|---|---|
| Form B of 2-(3-fluoro-4- | 15 |
| hydroxyphenyl)-7-vinyl-1,3- | |
| benzoxazol-5-ol | |
| Gelucire 44/14 | 40 |
| Labrasol | 40 |
| Polyvinylpyrrolidone (povidone | 5 |
| K25) | |

### Example 17

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 16.

### Example 18

A Semi-Solid Formulation of Form B of 2-(3-Fluoro-4-Hydroxyphenyl)-7-Vinyl-1,3-Benzoxazol-5-Ol

The semi-solid formulation is prepared by the procedure of Example 14 using the active ingredients in the percentages shown in Table 9

TABLE 9

| INGREDIENT | % WT/WT |
|---|---|
| Form B of 2-(3-fluoro-4- | 16.67 |
| hydroxyphenyl)-7-vinyl-1,3- | |
| benzoxazol-5-ol | |
| Gelucire 44/14 | 38.33 |
| Labrasol | 40 |
| Polyoxyethylene 20 Sorbitan | 5 |
| Monooleate (Tween 80) | |

### Example 19

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 18.

### Example 20

### Semi-Solid Formulation of Form B of 2-(3-Fluoro-4-Hydroxyphenyl)-7-Vinyl-1,3-Benzoxazol-5-Ol

The semi-solid formulation is prepared by the procedure of Example 14 using the active ingredients in the percentages shown in Table 10.

TABLE 10

| INGREDIENT | % WT/WT |
|---|---|
| Form B of 2-(3-fluoro-4- | 16.67 |
| hydroxyphenyl)-7-vinyl-1,3- | |
| benzoxazol-5-ol | |
| Gelucire 44/14 | 18.33 |
| Labrasol | 60 |
| Polyoxyethylene 20 Sorbitan | 5 |
| Monooleate (Tween 80) | |

### Example 21

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 20.

### Example 22

### Semi-Solid Formulation of Form B of 2-(3-Fluoro-4-Hydroxyphenyl)-7-Vinyl-1,3-Benzoxazol-5-Ol

The semi-solid formulation is prepared by the following procedure using the active ingredients in the percentages shown in Table 11.

1. Each of the active ingredients is weighed out independently.

2. The Gelucire 44/14 is placed in a mixer bowel that is then heated to 50 to 80° C. to melt the Gelucire 44/14.

3. The polyoxyethylene 20 sorbitan monooleate (Tween 80) is added to the mixture of step 2 and mixed.

4. Form B of 2-(3-fluoro-4-hydroxyphenyl)-7-vinyl-1,3-benzoxazol-5-ol is added to the mixture of step 3 and is mixed to suspend the crystal form (Form B).

TABLE 11

| INGREDIENT | % WT/WT |
|---|---|
| Form B of 2-(3-fluoro-4- | 16.67 |
| hydroxyphenyl)-7-vinyl-1,3- | |
| benzoxazol-5-ol | |
| Gelucire 44/14 | 78.33 |
| Polyoxyethylene 20 Sorbitan | 5 |
| Monooleate (Tween 80) | |

### Example 23

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 22.

### Example 24

### A Semi-Solid Formulation of Form B of 2-(3-Fluoro-4-Hydroxyphenyl)-7-Vinyl-1,3-Benzoxazol-5-Ol

The semi-solid formulation is prepared by the procedure of Example 14 using the active ingredients in the percentages shown in Table 12.

TABLE 12

| INGREDIENT | % WT/WT |
|---|---|
| Form B of 2-(3-fluoro-4- | 16.67 |
| hydroxyphenyl)-7-vinyl-1,3- | |
| benzoxazol-5-ol | |
| Gelucire 44/14 | 70 |
| Labrasol | 8.33 |
| Polyoxyethylene 20 Sorbitan | 5 |
| Monooleate (Tween 80) | |

### Example 25

### Hard Gel Capsule Containing a Semi-Solid Formulation of Form B of 2-(3-Fluoro-4-Hydroxyphenyl)-7-Vinyl-1,3-Benzoxazol-5-Ol

The hard gel capsule is prepared by the method of Example 9 using the semi-solid formulation of Example 24.

### Example A1

### Preparation of a Granule and Tablet Containing 75 Mg of Form B of 2-(3-Fluoro-4-Hydroxyphenyl)-7-Vinyl-1,3-Benzoxazol-5-Ol by a Wet Granulation Process

The solid pharmaceutical formulation is prepared by steps 1-7 of the procedure below, utilizing the weight/weight percentages (% wt/wt) of the ingredients shown in Table 17. The tablets are prepared by steps 8-10 of the procedure below. Each tablet contains the unit dose amounts shown in Table 13.

1. An aqueous solution of polyvinylpyrrolidone (povidone K25) and sodium lauryl sulfate is prepared in purified water.

2. Form B of 2-(3-fluoro-4-hydroxyphenyl)-7-vinyl-1,3-benzoxazol-5-ol is mixed with a portion of the mannitol (Pearlitol 200SD), passed through an appropriate screen and placed in a high shear mixer bowl.

3. The remainder of the mannitol, microcrystalline cellulose (Avicel pH 113), and croscarmellose sodium is passed through an appropriate screen into the mixer bowl and mixed.

4. The blend from step 3 is granulated using the step 1 solution.

5. The step 4 granulation is dried and passed through an appropriate screen.

6. The magnesium stearate is passed through an appropriate screen.

7. The magnesium stearate is premixed with an equal portion of the blend in step 5, then the premix is added to the remainder of the step 5 material and mixed in a blender.

8. The final blend from step 7 is compressed into tablets using a tablet press.

9. A 7.5% solid solution of Opaglos 2 is prepared.

10. A sufficient amount of coating solution is applied to the tablets in order to provide a 3.0% wt/wt increase in dried tablet weight.

TABLE 13

| | | UNIT DOSE |
|---|---|---|
| INGREDIENT | % WT/WT | (mg/tablet) |
| Form B of 2-(3-fluoro-4- | 25.0 | 75.0 |
| hydroxyphenyl)-7-vinyl-1,3- | | |
| benzoxazol-5-ol | | |
| Mannitol (Pearlitol 200SD)^{a} | 51.5 | 154.5 |
| Microcrystalline Cellulose (Avicel pH | 15.0 | 45.0 |
| 113) | | |
| Croscarmellose Sodium | 4.0 | 12.0 |
| Polyvinylpyrrolidone (Povidone K25) | 2.0 | 6.0 |
| Sodium Lauryl Sulfate | 2.0 | 6.0 |
| Magnesium Stearate | 0.5 | 1.5 |
| Purified Water^{b} | — | — |
| TOTAL | 100.0% | 300.0 |
| Film Coat | 3.0 | 9.0 |
| Opaglos 2, green | | |
| 97W11753 | | |

^{a}If assay is other than 100.0%, adjust the amount of input against mannitol accordingly.
^{b}Is used in the process, but does not appear in the final tablet product.

### Example A2

### A Solid Formulation and Tablet Containing 25 mg of Form B of 2-(3-Fluoro-4-Hydroxyphenyl)-7-Vinyl-1,3-Benzoxazol-5-Ol Made by a Wet Granulation Process

The solid pharmaceutical formulation is prepared by steps 1-7 of the procedure of Example A1, utilizing the weight/weight percentages (% wt/wt) of the ingredients shown in Table 18. The tablets are prepared by steps 8-10 of the procedure of Example A1. Each tablet contains the unit dose amounts shown in Table 14.

TABLE 14

| | | UNIT DOSE |
|---|---|---|
| INGREDIENT | % WT/WT | (mg/tablet) |
| Form B of 2-(3-fluoro-4- | 25.0 | 25.0 |
| hydroxyphenyl)-7-vinyl-1,3- | | |
| benzoxazol-5-ol | | |
| Mannitol (Pearlitol 200SD)^{a} | 51.5 | 51.5 |
| Microcrystalline Cellulose (Avicel pH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water^{b} | — | — |
| TOTAL | 100.0% | 100.0 |
| Film Coat | 3.0 | 3.0 |
| Opaglos 2, green | | |
| 97W11753 | | |

^{a}If assay is other than 100.0%, adjust the amount of input against mannitol accordingly.
^{b}Is used in the process, but does not appear in the final tablet product.

### Example A3

### A Solid Formulation and Tablet Containing 5 mg of Form B of 2-(3-Fluoro-4-Hydroxyphenyl)-7-Vinyl-1,3-Benzoxazol-5-Ol Made by a Wet Granulation Process

The solid pharmaceutical formulation is prepared by steps 1-7 of the procedure of Example A1, utilizing the weight/weight percentages (% wt/wt) of the ingredients shown in Table 19. The tablets are prepared by steps 8-10 of the procedure of Example A1. Each tablet contains the unit dose amounts shown in Table 15.

TABLE 15

| | | UNIT DOSE |
|---|---|---|
| INGREDIENT | % WT/WT | (mg/tablet) |
| Form B of 2-(3-fluoro-4- | 5.0 | 5.0 |
| hydroxyphenyl)-7-vinyl-1,3- | | |
| benzoxazol-5-ol | | |
| Mannitol (Pearlitol 200SD)^{a} | 71.5 | 71.5 |
| Microcrystalline Cellulose (Avicel pH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water^{b} | — | — |
| TOTAL | 100.0% | 300.0 |
| Film Coat | 3.0 | 3.0 |
| Opaglos 2, green | | |
| 97W11753 | | |

^{a}If assay is other than 100.0%, adjust the amount of input against mannitol accordingly.
^{b}Is used in the process, but does not appear in the final tablet product.

### Example A4

### A Solid Formulation and Tablet Containing 150 Mg of Form B of 2-(3-Fluoro-4-Hydroxyphenyl)-7-Vinyl-1,3-Benzoxazol-5-Ol Made by a Wet Granulation Process

The pharmaceutical formulation is prepared by steps 1-7 of the procedure of Example A1, utilizing the weight/weight percentages (% wt/wt) of the ingredients shown in Table 20. The tablets are prepared by steps 8-10 of the procedure of Example A1. Each tablet contains the unit dose amounts shown in Table 16.

TABLE 16

| | | UNIT DOSE |
|---|---|---|
| INGREDIENT | % WT/WT | (mg/tablet) |
| Form B of 2-(3-fluoro-4- | 25.0 | 150.0 |
| hydroxyphenyl)-7-vinyl-1,3- | | |
| benzoxazol-5-ol | | |
| Mannitol (Pearlitol 200SD)^{a} | 51.5 | 309.0 |
| Microcrystalline Cellulose (Avicel pH | 15.0 | 90.0 |
| 113) | | |
| Croscarmellose Sodium | 4.0 | 24.0 |
| Polyvinylpyrrolidone (Povidone K25) | 2.0 | 12.0 |
| Sodium Lauryl Sulfate | 2.0 | 12.0 |
| Magnesium Stearate | 0.5 | 3.0 |
| Purified Water^{b} | — | — |
| TOTAL | 100.0% | 600.0 |
| Film Coat | 3.0 | 18.0 |
| Opaglos 2, green | | |
| 97W11753 | | |

^{a}If assay is other than 100.0%, adjust the amount of input against mannitol accordingly.
^{b}Is used in the process, but does not appear in the final tablet product.

### Example A5

### A Tablet Containing 75 Mg of Form B of 2-(3-Fluoro-4-Hydroxyphenyl)-7-Vinyl-1,3-Benzoxazol-5-Ol

The pharmaceutical formulation and tablet of the example is prepared by the method of Example A1, substituting Opadry AMB, yellow for Opaglos 2, green.

### Example A6

### A Tablet Containing 5 Mg of Form B of 2-(3-Fluoro-4-Hydroxyphenyl)-7-Vinyl-1,3-Benzoxazol-5-Ol

The pharmaceutical formulation and tablet of the example is prepared by the method of Example A1 using the ingredient amounts of Example A2, substituting Opadry AMB, yellow for Opaglos 2, green.

### Example A7

### A Tablet Containing 25 Mg of Form B of 2-(3-Fluoro-4-Hydroxyphenyl)-7-Vinyl-1,3-Benzoxazol-5-Ol

The pharmaceutical formulation and tablet of the example is prepared by the method of Example A1 using the ingredient amounts of Example A3, substituting Opadry AMB, yellow for Opaglos 2, green.

### Example A8

### A Tablet Containing 150 Mg of Form B of 2-(3-Fluoro-4-Hydroxyphenyl)-7-Vinyl-1,3-Benzoxazol-5-Ol

The pharmaceutical formulation and tablet of the example is prepared by the method of Example A1 using the ingredient amounts of Example A4, substituting Opadry AMB, yellow for Opaglos 2, green.

### Example A9

### A Solid Formulation and Tablet Containing 25% by Weight of Form B of 2-(3-Fluoro-4-Hydroxyphenyl)-7-Vinyl-1,3-Benzoxazol-5-Ol Made by a Wet Granulation Process

The pharmaceutical formulation is prepared by steps 1-7 of the procedure of Example A1, utilizing the weight/weight percentages (% wt/wt) of the ingredients shown in Table 17. The tablets are prepared by steps 8-10 of the procedure of Example A1.

TABLE 17

| INGREDIENT | % WT/WT |
|---|---|
| Form B of 2-(3-fluoro-4- | 25.0 |
| hydroxyphenyl)-7-vinyl-1,3- | |
| benzoxazol-5-ol | |
| Mannitol (Pearlitol 200SD)^{a} | 48.5 |
| Microcrystalline Cellulose (Avicel pH | 15.0 |
| 113) | |
| Polyvinylpyrrolidone (Povidone K25) | 2.0 |
| Croscarmellose Sodium | 4.0 |
| Sodium Lauryl Sulfate | 5.0 |
| Magnesium Stearate | 0.5 |
| Purified Water^{b} | — |
| TOTAL | 100.0% |

^{a}If assay is other than 100.0%, adjust the amount of input against mannitol accordingly.
^{b}Is used in the process, but does not appear in the final tablet product.

### Example A10

### A Solid Formulation and Tablet Containing 25% by Weight of Form B of 2-(3-Fluoro-4-Hydroxyphenyl)-7-Vinyl-1,3-Benzoxazol-5-Ol Made by a Wet Granulation Process

The pharmaceutical formulation is prepared by steps 1-7 of the procedure of Example A1, utilizing the weight/weight percentages (% wt/wt) of the ingredients shown in Table 18. The tablets are prepared by steps 8-10 of the procedure of Example A1.

TABLE 18

| INGREDIENT | % WT/WT |
|---|---|
| Form B of 2-(3-fluoro-4- | 25.0 |
| hydroxyphenyl)-7-vinyl-1,3- | |
| benzoxazol-5-ol | |
| Mannitol (Pearlitol 200SD)^{a} | 51.5 |
| Microcrystalline Cellulose (Avicel pH | 15.0 |
| 113) | |
| Polyvinylpyrrolidone (Povidone K25) | 2.0 |
| Croscarmellose Sodium | 4.0 |
| Sodium Lauryl Sulfate | 2.0 |
| Magnesium Stearate | 0.5 |
| Purified Water^{b} | — |
| TOTAL | 100.0% |

^{a}If assay is other than 100.0%, adjust the amount of input against mannitol accordingly.
^{b}Is used in the process, but does not appear in the final tablet product.

### Example A11

### A Solid Formulation and Tablet Containing 25% by Weight of Form B of 2-(3-Fluoro-4-Hydroxyphenyl)-7-Vinyl-1,3-Benzoxazol-5-Ol Made by a Wet Granulation Process

The solid pharmaceutical formulation is prepared by steps 1-7 of the procedure of Example A1, utilizing the weight/weight percentages (% wt/wt) of the ingredients shown in Table 19. The tablets are prepared by steps 8-10 of the procedure of Example A1.

TABLE 19

| INGREDIENT | % WT/WT |
|---|---|
| Form B of 2-(3-fluoro-4- | 25.0 |
| hydroxyphenyl)-7-vinyl-1,3- | |
| benzoxazol-5-ol | |
| Mannitol (Pearlitol 200SD)^{a} | 53.5 |
| Microcrystalline Cellulose (Avicel pH | 15.0 |
| 113) | |
| Polyvinylpyrrolidone (Povidone K25) | 2.0 |
| Croscarmellose Sodium | 4.0 |
| Sodium Lauryl Sulfate | 0.0 |
| Magnesium Stearate | 0.5 |
| Purified Water^{b} | — |
| TOTAL | 100.0% |

^{a}If assay is other than 100.0%, adjust the amount of input against mannitol accordingly.
^{b}Is used in the process, but does not appear in the final tablet product.

### Example A12

### A Tablet Containing 25 mg of Form B of 2-(3-Fluoro-4-Hydroxyphenyl)-7-Vinyl-1,3-Benzoxazol-5-Ol Prepared by a Direct Blend Method

The pharmaceutical formulation of the example is prepared by the procedure below, using the weight/weight percentage amounts (% wt/wt) shown in Table 20.
1. Lactose anhydrous, microcrystalline cellulose (Avicel pH 112), croscarmellose sodium, sodium lauryl sulfate, silicon dioxide (Syloid 244), and Form B of 2-(3-fluoro-4-hydroxyphenyl)-7-vinyl-1,3-benzoxazol-5-ol is added to a blender and blended for five to ten minutes.
2. The magnesium stearate is added to the mixture of step 1 and blended for an additional two minutes.
3. The blend of step 2 is then compressed into tablets using a tablet press.

TABLE 20

| INGREDIENT | % WT/WT |
|---|---|
| Form B of 2-(3-fluoro-4- | 25.0 |
| hydroxyphenyl)-7-vinyl-1,3- | |
| benzoxazol-5-ol | |
| Lactose Anhydrous | 49.5 |
| Microcrystalline Cellulose (Avicel pH | 15.0 |
| 112) | |
| Croscarmellose Sodium | 4.0 |
| Sodium Lauryl Sulfate | 5.0 |
| Silicon dioxide (Syloid 244) | 1.0 |
| Magnesium Stearate | 0.5 |
| TOTAL | 100.0% |

### Example A13

### Tablet Containing 25% by Weight of Form B of 2-(3-Fluoro-4-Hydroxyphenyl)-7-Vinyl-1,3-Benzoxazol-5-Ol Prepared by a Dry Blend Method

The pharmaceutical formulation of the example is prepared by the procedure below, using the weight/weight percentages (% wt/wt) amounts shown in Table 21.
1. Lactose anhydrous, microcrystalline cellulose (Avicel pH 112), croscarmellose sodium, sodium lauryl sulfate, silicon dioxide (Syloid 244), sodium carbonate, and Form B of 2-(3-fluoro-4-hydroxyphenyl)-7-vinyl-1,3-benzoxazol-5-ol is added to a blender and blended for five to ten minutes.
2. The magnesium stearate is added to the mixture of step 1 and blended for an additional two minutes.
3. The blend of step 2 is then compressed into tablets using a tablet press.

TABLE 21

| INGREDIENT | % WT/WT |
|---|---|
| Form B of 2-(3-fluoro-4- | 25.0 |
| hydroxyphenyl)-7-vinyl-1,3- | |
| benzoxazol-5-ol | |
| Lactose Anhydrous | 47.5 |
| Microcrystalline Cellulose (Avicel pH | 14.4 |
| 112) | |
| Croscarmellose Sodium | 3.84 |
| Sodium Lauryl Sulfate | 4.8 |
| Sodium carbonate | 4.0 |
| Silicon dioxide (Syloid 244) | 0.96 |
| Magnesium Stearate | 0.5 |
| TOTAL | 100.0% |

### Examples A14-A31

### Preparation of Granule and Tablets Containing 25% by Weight of Form B of 2-(3-Fluoro-4-Hydroxyphenyl)-7-Vinyl-1,3-Benzoxazol-5-Ol by a Wet Granulation Process

The granule and tablets of Examples A14-A31 are prepared at a 300.0 g batch size by the following procedure using the weight/weight percentages of sodium lauryl sulfate (SLS), polyvinylpyrrolidone (PVP), croscarmellose sodium (Cros.Na), and microcrystalline cellulose (Avicel PH 113) as shown Table 22. The percentage of Form B of 2-(3-fluoro-4-hydroxyphenyl)-7-vinyl-1,3-benzoxazol-5-ol in each of Examples A14-A31 is 25.0% wt/wt. The percentage of magnesium stearate in the granule and tablets is 0.5%. The percentage of mannitol is varied for each example and is calculated by substracting the percentages of SLS, PVP, croscarmellose sodium, microcrystalline cellulose and magnesium stearate in the batch from 100%. The weight values of each ingredient is calculated by multiplying the weight/weight percentages by the total 300.0 g batch size.

1. Mannitol (Pearlitol 200 SD), microcrystalline cellulose (Avicel PH 113) sodium lauryl sulfate, croscarmellose sodium, polyvinylpyrrolidone (povidone K25), magnesium stearate, and Form B of 2-(3-fluoro-4-hydroxyphenyl)-7-vinyl-1,3-benzoxazol-5-ol are weighed out independently for a 300 gram batch.

2. A 10% solution of sodium lauryl sulfate and polyvinylpyrrolidone (povidone K25) is prepared by dissolving the sodium lauryl sulfate in purified water followed by the polyvinylpyrrolidone.

3. 73 g of mannitol (Pearlitol 200SD) is passed through #16 mesh screen directly into a Diosna granulator.

4. Form B of 2-(3-fluoro-4-hydroxyphenyl)-7-vinyl-1,3-benzoxazol-5-ol is bag blended with 36 g of mannitol.

5. The step 4 mixture is passed through #16 mesh screen directly into the granulator.

6. The remaining mannitol is passed through #16 mesh screen directly into a Gral granulator.

7. The microcrystalline cellulose (Avicel PH 113) is passed through #16 mesh screen directly into the granulator.

8. The croscarmellose sodium is passed through #16 mesh screen directly into the granulator.

9. The materials are dry blended for 2 minutes with plow set at low speed.

10. The blend with is granulated with the step 2 solution over a period of three minutes using a pump with the plow set at low speed and the chopper off.

11. The percentage of water required for granulation is calculated using the following equation:

$$\% Water = \frac{Water ( g ) \times 100}{Water ( g ) + weight of step 1 ingredients ( g )}$$

12. After the granulation is completed, the granulation is mixed for additional 30 seconds with the plow at low speed and the chopper on.

13. The granulation is fluid bed dried at the temperature at an inlet temperature as shown in the table below until an LOD of less than 1-2% is obtained for a sample analyzed using Computrac moisture analyzer at 100° C.

14. The dried granulation of step 13 is milled using Comil.

15. The step 14 material is transferred into a PK-blender and is blended for 5 minutes without intensifier bar activation.

16. Based on the yield in step 15, the amount of magnesium stearate required for final blend is calculated (theoretical amount for 3 kg batch is 1.5 g of magnesium stearate.

17. The magnesium stearate is passed through #20 mesh screen and is premixed with approximately equal amount of step 14 blend.

18. The premix is transferred to the PK-blender of step 15 and is blended for 2 minutes without intensifier bar activation.

19. The step 18 blend is stored under refrigeration with desiccant protected from light and moisture until compression is carried out.

20. The required amount of final blend of step 20 for tablet compression is weighed out.

21. To make the desired tablet, the blend of step 20 is compressed using a rotary press equipped with 0.225″×0.6″ modified caplet tooling adjusting the press as necessary to the specification given below.

### Tablet Characteristics

Tablet Weight: Target 300 mg±3.75% (288.75-311.25 mg)

### Tablet Hardness Target 10 Kp (Range 7-13 Kp)

TABLE 22^{a}

| | % | % | % | % | Drying temperature |
|---|---|---|---|---|---|
| Example | SLS | PVP | Cros.Na | Avicel PH 113 | (° C.) |
| A14 | 1 | 1 | 2 | 25 | 60 |
| A15 | 3 | 1 | 2 | 5 | 60 |
| A16 | 3 | 3 | 2 | 5 | 80 |
| A17 | 2 | 2 | 4 | 15 | 70 |
| A18 | 1 | 3 | 2 | 25 | 80 |
| A19 | 3 | 1 | 2 | 25 | 80 |
| A20 | 1 | 3 | 6 | 25 | 60 |
| A21 | 1 | 3 | 6 | 5 | 80 |
| A22 | 3 | 3 | 6 | 25 | 80 |
| A23 | 1 | 1 | 6 | 5 | 60 |
| A24 | 3 | 1 | 6 | 25 | 60 |
| A25 | 2 | 2 | 4 | 15 | 70 |
| A26 | 3 | 3 | 6 | 5 | 60 |
| A27 | 3 | 3 | 2 | 25 | 60 |
| A28 | 3 | 1 | 6 | 5 | 80 |
| A29 | 1 | 3 | 2 | 5 | 60 |
| A30 | 1 | 1 | 2 | 5 | 80 |
| A31 | 1 | 1 | 6 | 25 | 80 |

^{a}For each example: 25.0% wt/wt of Form B of 2-(3-fluoro-4-hydroxyphenyl)-7-vinyl-1,3-benzoxazol-5-ol; 0.5% wt/wt of magnesium stearate; and mannitol (Pearlitol 200SD) in each example is adjusted to bring total to 100% w/wt.

Various modifications of the invention, in addition to those described herein, will be apparent to those skilled in the art from the foregoing description. Such modifications are also intended to fall within the scope of the appended claims. Each reference cited in the present application, including patents, published applications, and journal articles, is hereby incorporated by reference in its entirety.

What is claimed is:

1. A liquid or semi-solid pharmaceutical formulation comprising:
(a) a first carrier component comprising from about 10% to about 99.99% by weight of said pharmaceutical formulation;
(b) an optional second carrier component, when present, comprising up to about 70% by weight of said pharmaceutical formulation;
(c) an optional emulsifying/solubilizing component, when present, comprising from about 0.01% to about 30% by weight of said pharmaceutical formulation;
(d) an optional anti-crystallization/solubilizing component, when present, comprising from about 0.01% to about 30% by weight of said pharmaceutical formulation; and
(e) an active pharmacological agent comprising from about 0.01% to about 80% of said pharmaceutical formulation, wherein said active pharmacological agent comprises the anhydrate crystal form (Form B) of 2-(3-fluoro-4-hydroxyphenyl)-7-vinyl-1,3-benzoxazol-5-ol.

2. A liquid or semi-solid pharmaceutical formulation comprising:
(a) a first carrier component comprising from about 10% to about 99.99% by weight of said pharmaceutical formulation;
(b) an optional second carrier component, when present, comprising up to about 70% by weight of said pharmaceutical formulation;
(c) an emulsifying/solubilizing component comprising from about 0.01% to about 30% by weight of said pharmaceutical formulation;
(d) an optional anti-crystallization/solubilizing component, when present, comprising from about 0.01% to about 30% by weight of said pharmaceutical formulation; and
(e) an active pharmacological agent comprising from about 0.01% to about 80% of said pharmaceutical formulation, wherein said active pharmacological agent comprises the anhydrate crystal form (Form B) of 2-(3-fluoro-4-hydroxyphenyl)-7-vinyl-1,3-benzoxazol-5-ol.

3. The liquid or semi-solid pharmaceutical formulation of claim 2, wherein:
(a) said first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil;
(b) said optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil;
(c) said emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil; and
(d) said optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

4. The liquid or semi-solid pharmaceutical formulation of claim 2, wherein:
(a) said first carrier component comprises one or more of lauroyl macrogol glycerides, caprylocaproyl macrogolglycerides, or polyethylene glycol;
(b) said optional carrier component, when present, comprises lauroyl macrogol glycerides or caprylocaproyl macrogolglycerides;
(c) said emulsifying/solubilizing component comprises polyethoxylated sorbitan ester; and
(d) said optional anti-crystallization/solubilizing component, when present, comprises polyvinylpyrrolidone.

5. The liquid or semi-solid pharmaceutical formulation of claim 2, wherein at least about 80% by weight of 2-(3-fluoro-4-hydroxyphenyl)-7-vinyl-1,3-benzoxazol-5-ol in said active pharmacological agent is present in said anhydrate crystal form (Form B).

6. The liquid or semi-solid pharmaceutical formulation of claim 2, wherein at least about 90% by weight of 2-(3-fluoro-4-hydroxyphenyl)-7-vinyl-1,3-benzoxazol-5-ol in said active pharmacological agent is present in said anhydrate crystal form (Form B).

7. The liquid or semi-solid pharmaceutical formulation of claim 2, wherein:
(a) said first carrier component comprises lauroyl macrogol glycerides;
(b) said optional second carrier component, when present, comprises caprylocaproyl macrogolglycerides;
(c) said emulsifying/solubilizing component comprises polyoxyethylene-20 sorbitan monooleate; and
(d) said optional anti-crystallization/solubilizing component, when present, comprises polyvinylpyrrolidone.

8. The liquid or semi-solid pharmaceutical formulation of claim 2, wherein:
(a) said first carrier component comprises from about 30% to about 90% by weight of said pharmaceutical formulation;
(b) said optional second carrier component, when present, comprises up to about 50% by weight of said pharmaceutical formulation;
(c) said emulsifying/solubilizing component comprises from about 0.1% to about 20% by weight of said pharmaceutical formulation;
(d) said optional anti-crystallization/solubilizing component, when present, comprises from about 0.1% to about 20% by weight of said pharmaceutical formulation; and
(e) said active pharmacological agent comprises from about 0.1% to about 50% by weight of said pharmaceutical formulation.

9. The liquid or semi-solid pharmaceutical formulation of claim 2, wherein:
(a) said first carrier component comprises from about 50% to about 90% by weight of said pharmaceutical formulation;
(b) said optional second carrier component, when present, comprises up to about 30% by weight of said pharmaceutical formulation;
(c) said emulsifying/solubilizing component comprises from about 0.1% to about 10% by weight of said pharmaceutical formulation;
(d) said optional anti-crystallization/solubilizing component, when present, comprises from about 0.1% to about 20% by weight of said pharmaceutical formulation; and
(e) said active pharmacological agent comprises from about 0.1% to about 50% by weight of said pharmaceutical formulation.

10. The liquid or semi-solid pharmaceutical formulation of claim 2, wherein:
(a) the first carrier component comprises from about 50% to about 70% by weight of the pharmaceutical formulation;
(b) the optional second carrier component, when present, comprises up to about 30% by weight of the pharmaceutical formulation;
(c) the emulsifying/solubilizing component comprises from about 0.1% to about 10% by weight of the pharmaceutical formulation;
(d) the optional anti-crystallization/solubilizing component, when present, comprises from about 0.1% to about 15% by weight of the pharmaceutical formulation; and
(e) the active pharmacological agent comprises from about 0.1% to about 40% by weight of the pharmaceutical formulation.

11. The liquid or semi-solid pharmaceutical formulation of claim 2, wherein:
(a) said first carrier component comprises from about 30% to about 50% by weight of said pharmaceutical formulation;
(b) said optional second carrier component, when present, comprises from about 30% to about 50% by weight of said pharmaceutical formulation;
(c) said emulsifying/solubilizing component comprises from about 0.1% to about 10% by weight of said pharmaceutical formulation;
(d) said optional anti-crystallization/solubilizing component, when present, comprises from about 0.1% to about 15% by weight of said pharmaceutical formulation; and
(e) said active pharmacological agent comprises from about 0.1% to about 40% by weight of said pharmaceutical formulation.

12. The liquid or semi-solid pharmaceutical formulation of claim 2, wherein:
(a) said first carrier component comprises from about 65% to about 85% by weight of said pharmaceutical formulation;
(b) said optional second carrier component, when present, comprises up to about 30% by weight of said pharmaceutical formulation;
(c) said emulsifying/solubilizing component comprises from about 0.1% to about 10% by weight of said pharmaceutical formulation;
(d) said optional anti-crystallization/solubilizing component, when present, comprises from about 0.1% to about 15% by weight of said pharmaceutical formulation; and
(e) said active pharmacological agent comprises from about 0.1% to about 40% by weight of said pharmaceutical formulation.

13. The liquid or semi-solid pharmaceutical formulation of claim 2, wherein:
(a) said first carrier component comprises from about 65% to about 85% by weight of said pharmaceutical formulation;
(b) said optional second carrier component, when present, comprises from about 5% to about 15% by weight of said pharmaceutical formulation;
(c) said emulsifying/solubilizing component comprises from about 0.1% to about 10% by weight of said pharmaceutical formulation;
(d) said optional anti-crystallization/solubilizing component, when present, comprises from about 0.1% to about 15% by weight of said pharmaceutical formulation; and
(e) said active pharmacological agent comprises from about 0.1% to about 40% by weight of said pharmaceutical formulation.

14. The liquid or semi-solid pharmaceutical formulation of claim 2, wherein:
(a) said first carrier component comprises from about 75% to about 85% by weight of said pharmaceutical formulation;
(b) said optional second carrier component, when present, comprises from about 5% to about 15% by weight of said pharmaceutical formulation;
(c) said emulsifying/solubilizing component comprises from about 2% to about 7% by weight of said pharmaceutical formulation;
(d) said optional anti-crystallization/solubilizing component, when present, comprises from about 2% to about 7% by weight of said pharmaceutical formulation; and
(e) said active pharmacological agent comprises from about 10% to about 20% by weight of said pharmaceutical formulation.

15. The liquid or semi-solid pharmaceutical formulation of claim 2, wherein:
(a) said first carrier component comprises from about 65% to about 75% by weight of said pharmaceutical formulation;
(b) said optional second carrier component, when present, comprises from about 5% to about 15% by weight of said pharmaceutical formulation;
(c) said emulsifying/solubilizing component comprises from about 2% to about 7% by weight of said pharmaceutical formulation;
(d) said optional anti-crystallization/solubilizing component, when present, comprises from about 2% to about 7% by weight of said pharmaceutical formulation; and
(e) said active pharmacological agent comprises from about 10% to about 20% by weight of said pharmaceutical formulation.

16. The liquid or semi-solid pharmaceutical formulation of claim 15, wherein:
(a) said first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil;
(b) said optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil;
(c) said emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil; and
(d) said optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

17. The liquid or semi-solid pharmaceutical formulation of claim 15, wherein:
(a) said first carrier component comprises one or more of lauroyl macrogol glycerides, caprylocaproyl macrogolglycerides, or polyethylene glycol;
(b) said optional carrier component, when present, comprises lauroyl macrogol glycerides or caprylocaproyl macrogolglycerides;
(c) said emulsifying/solubilizing component comprises polyethoxylated sorbitan ester; and
(d) said optional anti-crystallization/solubilizing component, when present, comprises polyvinylpyrrolidone.

18. The liquid or semi-solid pharmaceutical formulation of claim 15, wherein:
(a) said first carrier component comprises lauroyl macrogol glycerides;
(b) said optional second carrier component, when present, comprises caprylocaproyl macrogolglycerides;
(c) said emulsifying/solubilizing component comprises polyoxyethylene-20 sorbitan monooleate; and
(d) said optional anti-crystallization/solubilizing component, when present, comprises polyvinylpyrrolidone.

19. The liquid or semi-solid pharmaceutical formulation of claim 15, wherein at least about 80% by weight of 2-(3-fluoro-4-hydroxyphenyl)-7-vinyl-1,3-benzoxazol-5-ol in said active pharmacological agent is present in said anhydrate crystal form (Form B).

20. The liquid or semi-solid pharmaceutical formulation of claim 15, wherein said active pharmacological agent comprises at least about 90% by weight of the anhydrous crystal form of 2-(3-fluoro-4-hydroxyphenyl)-7-vinyl-1,3-benzoxazol-5-ol.

21. A hard gel or soft gel capsule comprising the liquid or semi-solid pharmaceutical formulation of claim 2.

22. A process for preparing the liquid or semi-solid pharmaceutical formulation of claim 2 comprising mixing said first carrier component and said active pharmaceutical agent with sufficient heating to obtain a suspension of said active pharmaceutical agent.

23. The process of claim 22 wherein said mixing is performed in a heated jacketed bowl.

24. The process of claim 22 wherein said first carrier is melted prior to said mixing.

25. The process of claim 22 further comprising mixing said first carrier component, said second optional carrier component, if present, said emulsifying/solubilizing component and said optional anti-crystallization/solubilizing component, if present, with sufficient heating to enable blending, prior to said mixing to form said suspension.

26. The process of claim 25 further comprising melting said optional second carrier component, said emulsifying/solubilizing component, and said optional anti-crystallization/solubilizing component prior to said mixing of said first carrier component, said optional second carrier component, said emulsifying/solubilizing component, and said optional anti-crystallization/solubilizing component.

27. The process of claim 25 further comprising adding said optional second carrier component, said emulsifying/solubilizing component, and said optional anti-crystallization/solubilizing component in separate stages to said first carrier component.

28. The process of claim 22 wherein:
(a) said first carrier component comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, glycerol, sorbic acid, sorbitol, or polyethoxylated vegetable oil;
(b) said optional second carrier component, when present, comprises one or more of lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyoxypropylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, squalene, hydrogenated polyisobutene, mineral oil, glycerol, sorbic acid, sorbitol, vegetable oil, or polyethoxylated vegetable oil;
(c) said emulsifying/solubilizing component comprises one or more of metallic alkyl sulfate, quaternary ammonium compounds, salts of fatty acids, sulfosuccinates, taurates, amino acids, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, polyoxyethylene fatty alcohol ether, fatty acid, polyethoxylated fatty acid ester, propylene glycol fatty acid ester, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil; and
(d) said optional anti-crystallization/solubilizing component, when present, comprises one or more of metallic alkyl sulfate, polyvinylpyrrolidone, lauroyl macrogol glycerides, caprylocaproyl macrogolglycerides, stearoyl macrogol glycerides, linoleoyl macrogol glycerides, oleoyl macrogol glycerides, polyalkylene glycol, polyethylene glycol, polypropylene glycol, polyoxyethylene-polyoxypropylene copolymer, fatty alcohol, polyoxyethylene fatty alcohol ether, fatty acid, polyethoxylated fatty acid ester, propylene glycol fatty acid ester, fatty ester, glycerides of fatty acid, polyoxyethylene-glycerol fatty ester, polyglycolized glycerides, polyglycerol fatty acid ester, sorbitan ester, polyethoxylated sorbitan ester, polyethoxylated cholesterol, polyethoxylated castor oil, polyethoxylated sterol, lecithin, or polyethoxylated vegetable oil.

29. The process of claim 22 wherein:
(a) said first carrier component comprises one or more of lauroyl macrogol glycerides, caprylocaproyl macrogolglycerides, or polyethylene glycol;
(b) said optional second carrier component, when present, comprises lauroyl macrogol glycerides or caprylocaproyl macrogolglycerides;
(c) said emulsifying/solubilizing component comprises polyethoxylated sorbitan ester; and
(d) said optional anti-crystallization/solubilizing component, when present, comprises polyvinylpyrrolidone.

30. The process of claim 22 wherein:
(a) said first carrier component comprises lauroyl macrogol glycerides;
(b) said optional second carrier component, when present, comprises caprylocaproyl macrogolglycerides;
(c) said emulsifying/solubilizing component comprises polyoxyethylene-20 sorbitan monooleate; and
(d) said optional anti-crystallization/solubilizing component, when present, comprises polyvinylpyrrolidone.

31. A product of a process of claim 22.

32. A process for preparing the liquid or semi-solid pharmaceutical formulation of claim 2 comprising mixing said first carrier component and said active pharmaceutical agent with sufficient heating to obtain a solution.

33. The process of claim 32 further comprising mixing said first carrier component, said second optional carrier component, if present, said emulsifying/solubilizing component and said optional anti-crystallization/solubilizing component, if present, with sufficient heating to enable blending, prior to said mixing to form said solution.

34. A solid pharmaceutical formulation comprising:
(a) a first diluent/filler component comprising from about 30% to about 95% by weight of said formulation;
(b) an optional second diluent/filler component, when present, comprising up to about 40% by weight of said pharmaceutical formulation;
(c) a disintegrant component comprising from about 0.01% to about 30% by weight of said pharmaceutical formulation;
(d) a binder component comprising from about 0.01% to about 20% by weight of said pharmaceutical formulation;
(e) a wetting agent component comprising from about 0.01% to about 20% by weight of said pharmaceutical formulation;
(f) an optional lubricant component, when present, comprising from about 0.01% to about 10% by weight of said pharmaceutical formulation; and
(g) an active pharmacological agent comprising from about 0.01% to about 80% by weight of said pharmaceutical formulation, wherein said active pharmacological agent comprises the anhydrate crystal form (Form B) of 2-(3-fluoro-4-hydroxyphenyl)-7-vinyl-1,3-benzoxazol-5-ol.

35. The solid pharmaceutical formulation of claim 34, wherein:
(a) said first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate;
(b) said second optional diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, pregelatinized starch, sodium starch glycolate, a calcium phosphate, a metal carbonate, a metal oxide, or a metal aluminosilicate;
(c) said disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate;
(d) said binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol;
(e) said wetting agent component comprises one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium; and
(f) said optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

36. The solid pharmaceutical formulation of claim 34, wherein:
(a) said first diluent/filler component comprises mannitol;
(b) said second optional diluent/filler component, when present, comprises microcrystalline cellulose;
(c) said disintegrant component comprises croscarmellose sodium;
(d) said binder component comprises polyvinylpyrrolidone;
(e) said wetting agent component comprises sodium lauryl sulfate; and
(f) said optional lubricant component, when present, comprises magnesium stearate.

37. The solid pharmaceutical formulation of claim 34, wherein at least about 80% by weight of 2-(3-fluoro-4-hydroxyphenyl)-7-vinyl-1,3-benzoxazol-5-ol in said active pharmacological agent is present in said anhydrate crystal form (Form B).

38. The solid pharmaceutical formulation of claim 34, wherein at least about 90% by weight of 2-(3-fluoro-4-hydroxyphenyl)-7-vinyl-1,3-benzoxazol-5-ol in said active pharmacological agent is present in said anhydrate crystal form (Form B).

39. The solid pharmaceutical formulation of claim 34 wherein:
(a) said first diluent/filler component comprises from about 40% to about 80% by weight of said formulation;
(b) said optional second diluent/filler component, when present, comprises up about 20% by weight of said pharmaceutical formulation;
(c) said disintegrant component comprises from about 0.1% to about 20% by weight of said pharmaceutical formulation;
(d) said binder component comprises from about 0.1% to about 10% by weight of said pharmaceutical formulation;
(e) said wetting agent component comprises from about 0.1% to about 10% by weight of said pharmaceutical formulation; and
(f) said optional lubricant component, when present, comprises from about 0.01% to about 5% by weight of said pharmaceutical formulation; and
(g) said active pharmacological agent comprises from about 0.1% to about 50% by weight of said pharmaceutical formulation.

40. The solid pharmaceutical formulation of claim 34 wherein:
(a) said first diluent/filler component comprises from about 40% to about 80% by weight of said pharmaceutical formulation;
(b) said optional second diluent/filler component, when present, comprises from about 10% to about 20% by weight of said pharmaceutical formulation;
(c) said disintegrant component comprises from about 1% to about 10% by weight of said pharmaceutical formulation;
(d) said binder component comprises from about 1% to about 8% by weight of said pharmaceutical formulation;
(e) said wetting agent component comprises from 1% to about 8% by weight of said pharmaceutical formulation;
(f) said optional lubricant component, when present, comprises from about 0.1% to about 2% by weight of said pharmaceutical formulation; and
(g) said active pharmacological agent comprises from about 1% to about 40% by weight of said pharmaceutical formulation.

41. The solid pharmaceutical formulation of claim 34 wherein:
(a) said first diluent/filler component comprises from about 60% to about 80% by weight of said pharmaceutical formulation;
(b) said optional second diluent/filler component, when present, comprises from about 10% to about 20% by weight of said pharmaceutical formulation;
(c) said disintegrant component comprises from about 2% to about 6% by weight of said pharmaceutical formulation;
(d) said binder component comprises from about 1% to about 3% by weight of said pharmaceutical formulation;
(e) said wetting agent component comprises from about 1% to about 3% by weight of said pharmaceutical formulation;
(f) said optional lubricant component, when present, comprises from about 0.1% to about 1% by weight of said pharmaceutical formulation; and
(g) said active pharmacological agent comprises from about 1% to about 10% by weight of said pharmaceutical formulation.

42. The solid pharmaceutical formulation of claim 34 wherein:
(a) said first diluent/filler component comprises from about 40% to about 60% by weight of said pharmaceutical formulation;
(b) said optional second diluent/filler component, when present, comprises from about 10% to about 20% by weight of said pharmaceutical formulation;
(c) said disintegrant component comprises from about 2% to about 6% by weight of said pharmaceutical formulation;
(d) said binder component comprises from about 1% to about 3% by weight of said pharmaceutical formulation;
(e) said wetting agent component comprises from about 1% to about 3% by weight of said pharmaceutical formulation;
(f) said optional lubricant component, when present, comprises from about 0.1% to about 1% by weight of said pharmaceutical formulation; and
(g) said active pharmacological agent comprises from about 10% to about 30% by weight of said pharmaceutical formulation.

43. The solid pharmaceutical formulation of claim 42, wherein:
(a) said first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate;
(b) said second optional diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, pregelatinized starch, sodium starch glycolate, a calcium phosphate, a metal carbonate, a metal oxide, or a metal aluminosilicate;
(c) said disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate;
(d) said binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol;
(e) said wetting agent component comprises one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium; and
(f) said optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

44. The solid pharmaceutical formulation of claim 42, wherein:
(a) said first diluent/filler component comprises mannitol;
(b) said second optional diluent/filler component, when present, comprises microcrystalline cellulose;
(c) said disintegrant component comprises croscarmellose sodium;
(d) said binder component comprises polyvinylpyrrolidone;
(e) said wetting agent component comprises sodium lauryl sulfate; and
(f) said optional lubricant component, when present, comprises magnesium stearate.

45. The solid pharmaceutical formulation of claim 42, wherein at least about 80% by weight of 2-(3-fluoro-4-hydroxyphenyl)-7-vinyl-1,3-benzoxazol-5-ol in said active pharmacological agent is present in said anhydrate crystal form (Form B).

46. The pharmaceutical formulation of claim 42, wherein at least about 90% by weight of 2-(3-fluoro-4-hydroxyphenyl)-7-vinyl-1,3-benzoxazol-5-ol in said active pharmacological agent is present in said anhydrate crystal form (Form B).

47. A process for preparing the solid pharmaceutical formulation of claim 34 comprising:
(a) mixing the active pharmacological agent with the first diluent/filler component, the disintegrant component, and the optional second filler/diluent component, if present, to form an initial mixture; and
(b) granulating said initial mixture with an aqueous solution comprising the wetting agent component to form a granulated mixture.

48. The process of claim 47 wherein (a) comprises:
(i) mixing said active pharmacological agent with at least a portion of said first diluent/filler component to form a first mixture; and
(ii) mixing said first mixture with the remainder of said first diluent/filler component, if any, said disintegrant component, and said optional second filler/diluent component, if present, to form said initial mixture.

49. The process of claim 47 wherein said aqueous solution further comprises the binder component.

50. The process of claim 47 further comprising:
(i) drying said granulated mixture to form a dried granulated mixture; and
(ii) mixing the optional lubricant component, if present, with said dried granulated mixture to form a final mixture.

51. The process of claim 50 wherein (ii) comprises:
(a) mixing said optional lubricant component, if present, with a portion of said dried granulated mixture; and
(b) mixing the mixture from (i) with the remainder of said dried granulated mixture.

52. The process of claim 51 wherein (b) is carried out in a blender.

53. The process of claim 47 comprising:
(i) mixing said active pharmacological agent with at least a portion of said first diluent/filler component to form a first mixture;
(ii) mixing said first mixture with the remainder of said first diluent/filler component, if any, said disintegrant component, and said optional second filler/diluent component, if present, to form said initial mixture;
(iii) granulating said initial mixture with an aqueous solution comprising the wetting agent component to form a granulated mixture;
(iv) drying said granulated mixture to form a dried granulated mixture;
(v) mixing the optional lubricant component, if present, with said at least a portion of said dried granulated mixture; and
(vi) mixing the mixture from (v) with the remainder of said dried granulated mixture, if any.

54. The process of claim 53 wherein said aqueous solution further comprises the binder component.

55. The process of claim 47 wherein:
(a) said first diluent/filler component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate;
(b) said second optional diluent/filler component, when present, comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, pregelatinized starch, sodium starch glycolate, a calcium phosphate, a metal carbonate, a metal oxide, or a metal aluminosilicate;
(c) said disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate;
(d) said binder component comprises one or more of polyvinylpyrrolidone, copovidone, hydroxypropylcellulose, hydroxypropylmethylcellulose, crosslinked poly(acrylic acid), gum arabic, gum acacia, gum tragacanath, lecithin, casein, polyvinyl alcohol, gelatin, kaolin, cellulose, methylcellulose, hydroxymethylcellulose, carboxymethylcellulose, carboxymethylcellulose calcium, carboxymethylcellulose sodium, hydroxypropylcellulose, hydroxypropylmethylcellulose phthalate, hydroxyethylcellulose, methylhydroxyethylcellulose, silicified microcrystalline cellulose, starch, maltodextrin, dextrins, microcrystalline cellulose, or sorbitol;
(e) said wetting agent component comprises one or more of metallic lauryl sulfate, polyethylene glycol, glycerides of fatty ester, polyoxyethylene-polyoxypropylene copolymer, polyoxyethylene-alkyl ether, metal alkyl sulfate, polyoxyethylene sorbitan fatty acid ester, polyoxyethylene castor oil derivative, sugar ester of fatty acid, polyglycolized glyceride, quaternary ammonium amine compound, lauroyl macrogol glycerides, caprylocaproyl macrogolglycerides, stearoyl macrogol glycerides, linoleoyl macrogol glycerides, oleoyl macrogol glycerides, polyethoxylated vegetable oil, polyethoxylated sterol, polyethoxylated cholesterol, polyethoxylated glycerol fatty acid ester, polyethoxylated fatty acid ester, sulfosuccinate, taurate, or docusate sodium; and
(f) said optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethylene glycol, polypropylene glycol, polyalkylene glycol, or sodium chloride.

56. The process of claim 47 wherein:
(a) said first diluent/filler component comprises mannitol;
(b) said second optional diluent/filler component, when present, comprises microcrystalline cellulose;
(c) said disintegrant component comprises croscarmellose sodium;
(d) said binder component comprises polyvinylpyrrolidone;
(e) said wetting agent component comprises sodium lauryl sulfate; and
(f) said optional lubricant component, when present, comprises magnesium stearate.

57. A product of a process of claim 47.

58. A process for producing the solid pharmaceutical formulation of claim 34 comprising:
(i) mixing said first diluent/filler component, said optional second diluent/filler component, if present, said disintegrant component, said binder component, said wetting agent component, and said active pharmacological agent to form a first mixture; and
ii) optionally granulating said first mixture.

59. The process of claim 58 wherein said first mixture further comprises the optional lubricant component.

60. A product of a process of claim 58.

61. A tablet comprising the solid pharmaceutical formulation of claim 34.

62. A process for producing a tablet comprising compressing the solid pharmaceutical formulation of claim 34.

63. The process of claim 62 further comprising milling said pharmaceutical formulation prior to said compressing of the pharmaceutical formulation
